(19) United States
(12) Patent Application Publication
(10) Pub. No.: US 2024/0177807 A1
(43) Pub. Date: May 30, 2024

(54) CLUSTER SEGMENTATION AND CONDITIONAL BASE CALLING

(71) Applicant: Illumina, Inc., San Diego, CA (US)

(72) Inventors: John S. Vieceli, San Diego, CA (US); Eric Jon Ojard, San Francisco, CA (US); Aathavan Karunakaran, Berkeley, CA (US); David Olmstead Bracher, San Diego, CA (US); Gery Vessere, San Francisco, CA (US)

(21) Appl. No.: 18/468,333

(22) Filed: Sep. 15, 2023

Related U.S. Application Data
(60) Provisional application No. 63/407,605, filed on Sep. 16, 2022.

(51) Int. Cl. G16B 40/00; G16B 30/00

(52) U.S. Cl. CPC G16B 40/00; G16B 30/00

(57) ABSTRACT

The technology disclosed is directed to cluster segmentation and base calling. The technology disclosed describes a computer-implemented method including segmenting a population of clusters into a plurality of subpopulations of clusters based on one or more prior bases called at one or more prior sequencing cycles of a sequencing run. At a current sequencing cycle of the sequencing run, the method includes applying a mixture of four distributions to current sequenced data of each subpopulation of clusters in the plurality of subpopulations of clusters, the four distributions corresponding to four bases adenine (A), cytosine (C), guanine (G), and thymine (T), and the current sequenced data being generated at the current sequencing cycle. The method further includes base calling clusters in a particular subpopulation of clusters using a corresponding mixture of four distributions.

### CROSS-REFERENCE TO RELATED APPLICATIONS

The present application claims the benefit of, and priority to, U.S. Provisional Application No. 63/407,605, entitled “CLUSTER SEGMENTATION AND CONDITIONAL BASE-CALLING,” filed on Sep. 16, 2022. The aforementioned application is hereby incorporated by reference in its entirety.

### FIELD OF THE TECHNOLOGY DISCLOSED

The technology disclosed relates to apparatus and corresponding methods for the automated analysis of an image or recognition of a pattern. Included herein are systems that transform an image for the purpose of (a) enhancing its visual quality prior to recognition, (b) locating and registering the image relative to a sensor or stored prototype, or reducing the amount of image data by discarding irrelevant data, and (c) measuring significant characteristics of the image. In particular, the technology disclosed relates to segmenting clusters into subpopulations and base calling clusters in a particular subpopulation.

### INCORPORATIONS

The following are incorporated by reference for all purposes as if fully set forth herein:

U.S. Nonprovisional patent application Ser. No. 17/308,035, titled “EQUALIZATION-BASED IMAGE PROCESSING AND SPATIAL CROSSTALK ATTENUATOR,” filed May 4, 2021 (Attorney Docket No. ILLM 1032-2/IP-1991-US);

U.S. Provisional Patent Application No. 63/106,256, titled “SYSTEMS AND METHODS FOR PER-CLUSTER INTENSITY CORRECTION AND BASE CALLING,” filed on Oct. 27, 2020;

U.S. Nonprovisional patent application Ser. No. 15/909,437, titled “OPTICAL DISTORTION CORRECTION FOR IMAGED SAMPLES,” filed on Mar. 1, 2018;

U.S. Nonprovisional patent application Ser. No. 14/530,299, titled “IMAGE ANALYSIS USEFUL FOR PATTERNED OBJECTS,” filed on Oct. 31, 2014;

U.S. Nonprovisional patent application Ser. No. 15/153,953, titled “METHODS AND SYSTEMS FOR ANALYZING IMAGE DATA,” filed on Dec. 3, 2014;

U.S. Nonprovisional patent application Ser. No. 15/863,241, titled “PHASING CORRECTION,” filed on Jan. 5, 2018;

U.S. Nonprovisional patent application Ser. No. 14/020,570, titled “CENTROID MARKERS FOR IMAGE ANALYSIS OF HIGH DENSITY CLUSTERS IN COMPLEX POLYNUCLEOTIDE SEQUENCING,” filed on Sep. 6, 2013;

U.S. Nonprovisional patent application Ser. No. 12/565,341, titled “METHOD AND SYSTEM FOR DETERMINING THE ACCURACY OF DNA BASE IDENTIFICATIONS,” filed on Sep. 23, 2009;

U.S. Nonprovisional patent application Ser. No. 12/295,337, titled “SYSTEMS AND DEVICES FOR SEQUENCE BY SYNTHESIS ANALYSIS,” filed on Mar. 30, 2007;

U.S. Nonprovisional patent application Ser. No. 12/020,739, titled “IMAGE DATA EFFICIENT GENETIC SEQUENCING METHOD AND SYSTEM,” filed on Jan. 28, 2008;

U.S. Nonprovisional patent application Ser. No. 13/833,619, titled “BIOSENSORS FOR BIOLOGICAL OR CHEMICAL ANALYSIS AND SYSTEMS AND METHODS FOR SAME,” filed on Mar. 15, 2013, (Attorney Docket No. IP-0626-US);

U.S. Nonprovisional patent application Ser. No. 15/175,489, titled “BIOSENSORS FOR BIOLOGICAL OR CHEMICAL ANALYSIS AND METHODS OF MANUFACTURING THE SAME,” filed on Jun. 7, 2016, (Attorney Docket No. IP-0689-US);

U.S. Nonprovisional patent application Ser. No. 13/882,088, titled “MICRODEVICES AND BIOSENSOR CARTRIDGES FOR BIOLOGICAL OR CHEMICAL ANALYSIS AND SYSTEMS AND METHODS FOR THE SAME,” filed on Apr. 26, 2013, (Attorney Docket No. IP-0462-US);

U.S. Nonprovisional patent application Ser. No. 13/624,200, titled “METHODS AND COMPOSITIONS FOR NUCLEIC ACID SEQUENCING,” filed on Sep. 21, 2012, (Attorney Docket No. IP-0538-US);

U.S. Nonprovisional patent application Ser. No. 13/006,206, titled “DATA PROCESSING SYSTEM AND METHODS,” filed on Jan. 13, 2011;

U.S. Nonprovisional patent application Ser. No. 15/936,365, titled “DETECTION APPARATUS HAVING A MICROFLUOROMETER, A FLUIDIC SYSTEM, AND A FLOW CELL LATCH CLAMP MODULE,” filed on Mar. 26, 2018;

U.S. Nonprovisional patent application Ser. No. 16/567,224, titled “FLOW CELLS AND METHODS RELATED TO SAME,” filed on Sep. 11, 2019;

U.S. Nonprovisional patent application Ser. No. 16/439,635, titled “DEVICE FOR LUMINESCENT IMAGING,” filed on Jun. 12, 2019;

U.S. Nonprovisional patent application Ser. No. 15/594,413, titled “INTEGRATED OPTOELECTRONIC READ HEAD AND FLUIDIC CARTRIDGE USEFUL FOR NUCLEIC ACID SEQUENCING,” filed on May 12, 2017;

U.S. Nonprovisional patent application Ser. No. 16/351,193, titled “ILLUMINATION FOR FLUORESCENCE IMAGING USING OBJECTIVE LENS,” filed on Mar. 12, 2019;

U.S. Nonprovisional patent application Ser. No. 12/638,770, titled “DYNAMIC AUTOFOCUS METHOD AND SYSTEM FOR ASSAY IMAGER,” filed on Dec. 15, 2009;

U.S. Nonprovisional patent application Ser. No. 13/783,043, titled “KINETIC EXCLUSION AMPLIFICATION OF NUCLEIC ACID LIBRARIES,” filed on Mar. 1, 2013; and

U.S. Nonprovisional patent application Ser. No. 16/826,168, titled “ARTIFICIAL INTELLIGENCE-BASED SEQUENCING,” filed 21 Mar. 2020 (Attorney Docket No. ILLM 1008-20/IP-1752-PRV).

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

This disclosure relates to analyzing image data to base call clusters during a sequencing run. One challenge with the analysis of image data is variation in intensity profiles of clusters in a cluster population being base called. This causes a drop in data throughput and an increase in error rate of base calling during the sequencing run.

There are many potential reasons for inter-cluster intensity profile variation. It may result from differences in cluster brightness, caused by fragment length distribution in the cluster population. It may result from phasing, which occurs when a molecule in a cluster does not incorporate a nucleotide in some sequencing cycles and lags behind other molecules, or when a molecule incorporates more than one nucleotide in a single sequencing cycle. It may result from fading, i.e., an exponential decay in signal intensity of clusters as a function of sequencing cycle number due to excessive washing and laser exposure as the sequencing run progresses. It may result from underdeveloped cluster colonies, i.e., small cluster sizes that produce empty or partially filled wells on a patterned flow cell. It may result from overlapping cluster colonies caused by unexclusive amplification. It may result from under-illumination or uneven-illumination, for example, due to clusters being located on edges of a flow cell. It may result from impurities on a flow cell that obfuscate emitted signal. It may result from polyclonal clusters, i.e., when multiple clusters are deposited in the same well.

One approach of reducing inter-cluster intensity profile variation and thus, reducing error rates in base calling is to segment clusters based on spatial regions. For example, when clusters are located in a flow cell containing a plurality of non-overlapping regions called “tiles”, clusters located on each tile can be processed together and any statistically derived quantities are from the clusters on that tile. One potentially challenge is the number of clusters per tile is typically on the order of hundreds of thousands to millions and thus, the intensities of the clusters on each tile may still vary significantly.

An opportunity arises to correct the inter-cluster intensity profile variation. Improved base calling throughput and reduced base calling error rate during the sequencing run may result.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which:

FIG. 1 depicts an example flow cell where clusters are immobilized and base called

during a sequencing process;

FIG. 2 illustrates an example of inter-cluster intensity profile variation discovered and corrected by the technology disclosed;

FIG. 3 illustrates an example workflow of segmenting a population of clusters into subpopulations based on segmentation conditions and separately base calling clusters on a subpopulation-by-subpopulation basis;

FIG. 4 illustrates an example 400 of how a mixture of intensity distributions fits the intensity profiles of a target cluster for base calling at a current sequencing cycle;

FIG. 5 illustrates various examples of condition determination logic 500 that determine the segmentation conditions for a population of clusters;

FIGS. 6A-6D illustrate examples of variations caused by prior base context in the intensity distributions of clusters;

FIG. 7 illustrates the intensity distributions of clusters with different insert lengths;

FIG. 8 illustrates another example workflow of segmenting a population of clusters into subpopulations based on segmentation conditions and separately base calling clusters on a subpopulation-by-subpopulation basis;

FIG. 9 illustrates sixteen subpopulations based on two prior base context;

FIG. 10 illustrates sixty-four subpopulations based on three prior base context;

FIG. 11A-11D illustrate example mixtures of four intensity distributions of clusters with

different SNR ratios.

FIGS. 12A-12B illustrates an example scaling logic that generates the intensity distributions representing the clusters with different SNR ratio profiles;

FIG. 13 illustrates an example workflow of resegmenting a population of clusters into subpopulations at different sequencing cycles;

FIG. 14 illustrates another example workflow of resegmenting a population of clusters into subpopulations at different sequencing cycles;

FIG. 15 illustrates an example high-dimensional mixture of intensity distributions;

FIG. 16 illustrates another example high-dimensional mixture of intensity distributions;

FIG. 17 illustrates an example workflow of correcting the intensity profiles of clusters at current sequencing cycle based on prior base context identified at prior sequencing cycles;

FIG. 18 illustrates another example workflow of correcting the intensity profiles of clusters at current sequencing cycle based on prior base context identified at prior sequencing cycles;

FIG. 19 illustrates an example comparison of the intensity profiles of clusters before and after correction;

FIG. 20 illustrates the performance results of base calling at 150 sequencing cycles at a sequencing run, by segmenting a population of clusters based on a single prior base call and two prior base calls;

FIG. 21 illustrates when soft-clipping errors are removed, the error rate of base calling conditioned on prior base context is significantly reduced;

FIG. 22 illustrates performance results of base calling conditioned on SNR ratio profiles of clusters;

FIG. 23 illustrates performance results of base calling in error rate and entropy conditioned on SNR ratio profiles of clusters;

FIG. 24 illustrates the correlation between the selected SNR ratio ranges and the error rate of base calling;

FIG. 25A illustrates the intensity profiles of clusters within each of the sixty-four subpopulations captured at the first intensity channel (e.g., blue channel) over a plurality of sequencing cycles;

FIG. 25B illustrates the offset values corresponding to the sixty-four subpopulations at the first intensity channel by applying the median intensity profile;

FIG. 26A illustrates the intensity profiles of clusters within each of the sixty-four subpopulations captured at the second intensity channel (e.g., green channel) over a plurality of sequencing cycles;

FIG. 26B illustrates the offset values corresponding to the sixty-four cluster subpopulations at the second intensity channel by applying the median intensity profile;

FIG. 27 illustrates the intensity correlation between two intensity channels for each of the sixty-four subpopulations;

FIG. 28A illustrates the intensity deviation caused by prior trimer context of the clusters that are called as base A and the clusters that are called as base T;

FIG. 28B illustrates the intensity deviation caused by prior trimer context of the clusters that are called as base A and the clusters that are called as base C;

FIG. 29 illustrates the performance results of base calling when correcting for prior base context;

FIG. 30 illustrates fractional MMR improvement when correcting for prior base context, by correlating the fractional MMR increase with deviations from median A intensity in the second intensity channel; and

FIG. 31 illustrates a computer system 3100 that can be used to implement the technology disclosed.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

The discussion is organized as follows. First, we introduce base calling clusters and inter-cluster intensity profile variations. Then we propose the technology disclosed for segmenting clusters into subpopulations based on their particular conditions and base calling these clusters separately on a subpopulation-by-subpopulation basis. We introduce a variety of segmentation conditions, including prior base context and other conditions related to the characteristics of clusters, followed by segmentations and base calling clusters within each subpopulation using a corresponding mixture of intensity distributions for four bases A, G, C and T. After that, we setup an example of high-dimensional mixtures of intensity distributions for simultaneously base calling clusters at current sequencing cycles and prior sequencing cycles. Advancing further, we give an example of measuring offset values corresponding to different prior base context and correcting the parameters of the corresponding mixtures of intensity distributions for base calling.

### Introduction

The technology disclosed begins with the concept of clusters, intensity extraction and base calling clusters. In one implementation, a sequencer uses sequencing by synthesis (SBS) technology for generating sequencing images. SBS relies on growing nascent strands complementary to cluster strands with fluorescently-labeled nucleotides, while tracking the emitted signal of each newly added nucleotide. The fluorescently-labeled nucleotides have a 3′ removable block that anchors a fluorophore signal of the nucleotide type. SBS occurs in repetitive sequencing cycles, each comprising three steps: (a) extension of a nascent strand by adding the fluorescently-labeled nucleotide; (b) excitation of the fluorophore using one or more lasers of an optical system of the sequencer and imaging through different filters of the optical system, yielding sequencing images; and (c) cleavage of the fluorophore and removal of the 3′ block in preparation for the next sequencing cycle. Incorporation and imaging are repeated up to a designated number of sequencing cycles, defining the read length, which refers to the number of base pairs (bp) sequenced from a DNA fragment. Using this approach, each sequencing cycle interrogates a new position along the cluster strands.

Intensity values can be extracted from different color/intensity channel sequencing images generated by a sequencer at each sequencing cycle during a sequencing run. Examples of the sequencer include Illumina's iSeq, HiSeqX, HiSeq 3000, HiSeq 4000, HiSeq 2500, NovaSeq 6000, NextSeq 550, NextSeq 1000, NextSeq 2000, NextSeqDx, MiSeq, and MiSeqDx.

The tremendous power of Illumina's sequencers stems from their ability to simultaneously execute and sense millions or even billions of analytes (e.g., clusters). A cluster comprises approximately one thousand identical copies of a template strand, though clusters vary in size and shape. Clusters are grown from the template strand, prior to the sequencing run, by bridge amplification or exclusion amplification of the input library which is a collection of similarly sized DNA fragments. The purpose of the amplification and cluster growth is to increase the intensity of the emitted signal since the imaging device cannot reliably sense fluorophore signal of a single strand. On the other hand, the imaging device perceives a cluster of thousands of template strands as a single spot, because the physical distance among the strands within the cluster is small.

The sequencing process occurs in a flow cell—a small glass slide that holds the input DNA fragments during the sequencing process. The flow cell is connected to the high-throughput optical system that includes microscopic imaging, excitation lasers, and fluorescence filters. An imaging device (e.g., a solid-state imager such as a charge-coupled device (CCD) or a complementary metal-oxide-semiconductor (CMOS) sensor) in the sequencer takes images at multiple locations along a series of non-overlapping regions called tiles. At each sequencing cycle, the imaging device takes sequencing images of each tile at each color/intensity channel. The sequence data of clusters immobilized on each tile at each sequencing cycle therefore includes intensity signals extracted from the sequencing images.

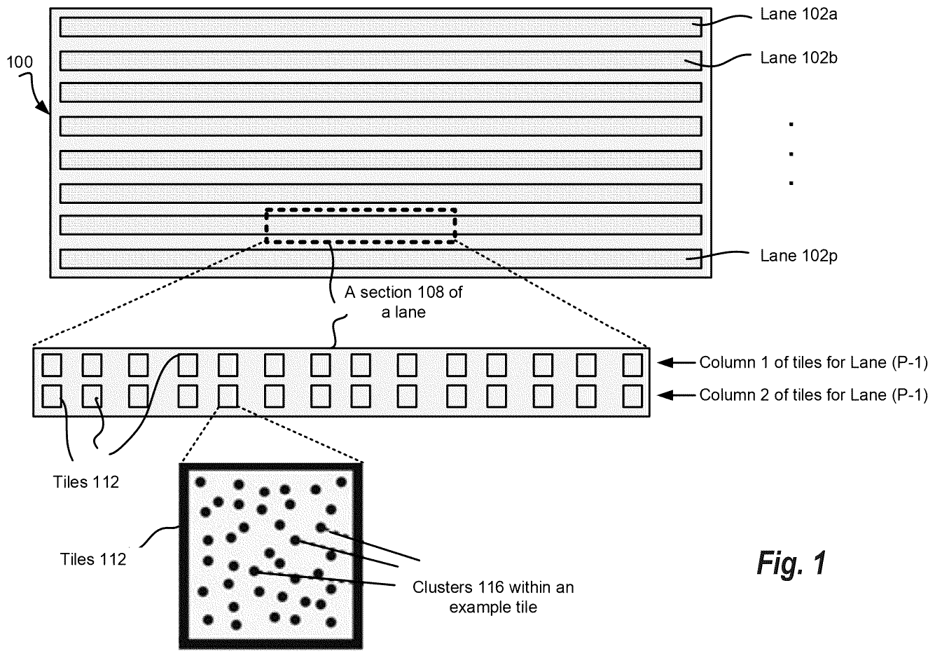

FIG. 1 depicts an example flow cell where clusters are immobilized and base called during a sequencing process. In one implementation, the flow cell 100 is partitioned in a plurality of chambers called lanes, such as lanes 102a, 102b, . . . , 102p, i.e., p represents a number of lanes. The lanes are physically separated from each other and may contain different tagged sequencing input libraries, distinguishable without sample cross contamination. Each individual lane 102 can further be partitioned into non-overlapping regions called “tiles” 112. For example, FIG. 1 illustrates a magnified view of a section 108 of an example lane. The section 108 is illustrated to comprise a plurality of tiles 112. Hundreds of thousands to millions of clusters 116 can be immobilized on the surface of each tile. At each sequencing cycle of a sequencing run, the imaging device of the sequence takes sequencing images of each tile at each color/intensity channel. The intensity profiles of clusters being base called at each sequencing cycle are extracted from the sequencing images and analyzed for base calling.

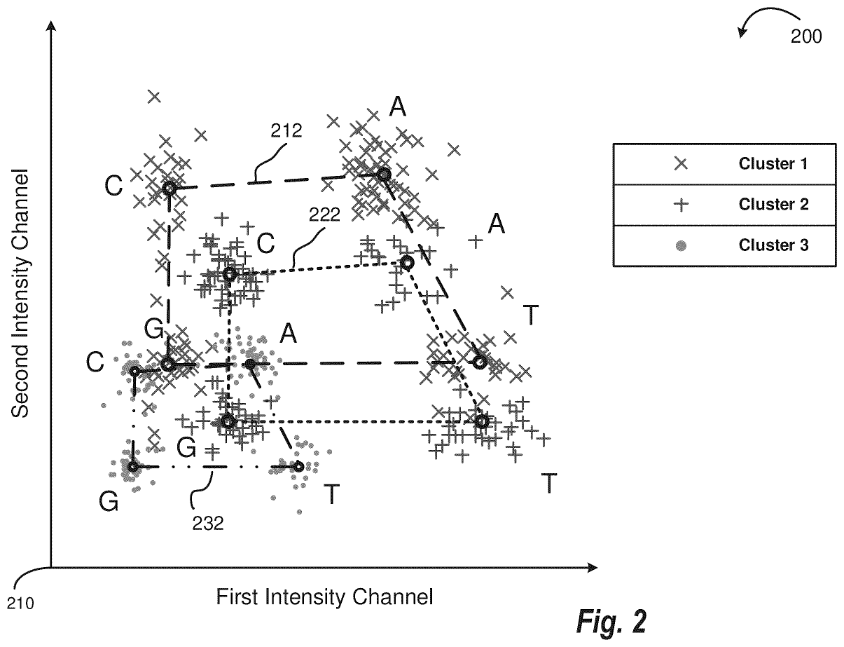

FIG. 2 illustrates an example of the inter-cluster intensity profile variation discovered and corrected by the technology disclosed. FIG. 2 depicts intensity profiles 212, 222, and 232 of clusters 1, 2, and 3 in a cluster population, respectively. Intensity profile of a target cluster comprises intensity values that capture the chemiluminescent signals produced due to nucleotide incorporations in the target cluster at a plurality of sequencing cycles during a sequencing run.

In FIG. 2, the “ [symbol] ” symbol represents the intensity values for cluster 1, the “ [symbol] ” symbol represents the intensity values for cluster 2, and the “●” symbol represents the intensity values for cluster 3. Each data point represents the intensity profiles of the corresponding cluster at a given sequencing cycle. The identity of the four different nucleotide types/bases A, G, C and T is encoded as a combination of the intensity values in two-color images, i.e., the first and second intensity channels. For example, a nucleic acid can be sequenced by providing a first nucleotide type (e.g., base T) that is detected at the first intensity channel (x-axis of the multi-dimensional space 200), a second nucleotide type (e.g., base C) that is detected at the second intensity channel (y-axis of the multi-dimensional space 200), a third nucleotide type (e.g., base A) that is detected at both the first and the second intensity channels, and a fourth nucleotide type (e.g., base G) that lacks a label that is not, or minimally, detected at either intensity channel.

In some implementations, the intensity profile is generated by iteratively fitting four intensity distributions (e.g., Gaussian distributions) to the intensity values in the first and the second intensity channels. The four intensity distributions correspond to the four bases A, C, T, and G. In the intensity profile, the intensity values in the first intensity channel are plotted against the intensity values in the second intensity channel (e.g., as a scatterplot), and the intensity values segregate into the four intensity distributions.

The intensity profiles can take any shape (e.g., trapezoids, squares, rectangles, rhombus, etc.). Analysis revealed that the intensity profiles of clusters take similar form (e.g., trapezoids), but differ in scale and shifts from an origin 210 of a multi-dimensional space 200. We refer to this as “inter-cluster intensity profile variation.” The multi-dimensional space 200 can be a cartesian space, a polar space, a cylindrical space, or a spherical space. Additional details about how the four intensity distributions are fitted to the intensity values for base calling can be found in U.S. Patent Application Publication No. 2018/0274023 A1, the disclosure of which is incorporated herein by reference in its entirety.

In one implementation, each intensity channel corresponds to one of a plurality of filter wavelength bands used by the optical system. In another implementation, each intensity channel corresponds to one of a plurality of imaging events at a sequencing cycle. In yet another implementation, each intensity channel corresponds to a combination of illumination with a specific laser and imaging through a specific optical filter of the optical system.

It would be apparent to one skilled in the art that the technology disclosed can be analogously applied to sequencing images generated using one-channel implementation, four-channel implementation, and so on.

As illustrated in FIG. 2, different clusters (e.g., cluster 1, cluster 2 and cluster 3) have different intensity profiles. Various conditions can contribute to the inter-cluster variations in the intensity profiles, which in turn increases error rate of base calling. For example, sequence-specific context identified at prior sequencing cycles and signal qualities of the intensity profiles may vary at each sequencing cycle. Other conditions can relate to the characteristics of clusters irrespective of prior base calls, including the profiles of genomic samples that are used to prepare the sequencing input library, adaptors that are attached to the template sequence prior to the cluster generation, lengths of template sequences, sizes and shapes of clusters, and spatial configurations/locations of clusters, etc. It is therefore important to identify different conditions that may cause inter-cluster intensity profile variations and take them into consideration during base calling, in order to minimize the inter-cluster intensity profile variation and reduce error rate of base calling.

The technology disclosed provides approaches of base calling clusters based on the different conditions associated with the clusters. In one implementation, the technology disclosed provides a condition determination logic that identifies the different conditions associated with the clusters, and a segmentation logic that segments clusters into a plurality of cluster subpopulations based on the identified segmentation conditions.

For a target cluster within a given subpopulation of clusters, a mixture of four intensity distributions corresponding to four bases adenine (A), cytosine (C), guanine (G) and thymine (T) can be applied to the intensity profiles of the target cluster for base calling. The mixture of four intensity distributions is generated by analyzing the intensity profiles of all clusters within the given subpopulation and thus, corresponds to the subpopulation. That is, each subpopulation includes clusters with similar conditions, and has a corresponding mixture of four intensity distributions used to base call the clusters within this subpopulation. By segmenting clusters by different conditions and separately base calling these clusters on a subpopulation-by-subpopulation basis, the technology disclosed reduces inter-cluster intensity variations which in turn reduces error rate.

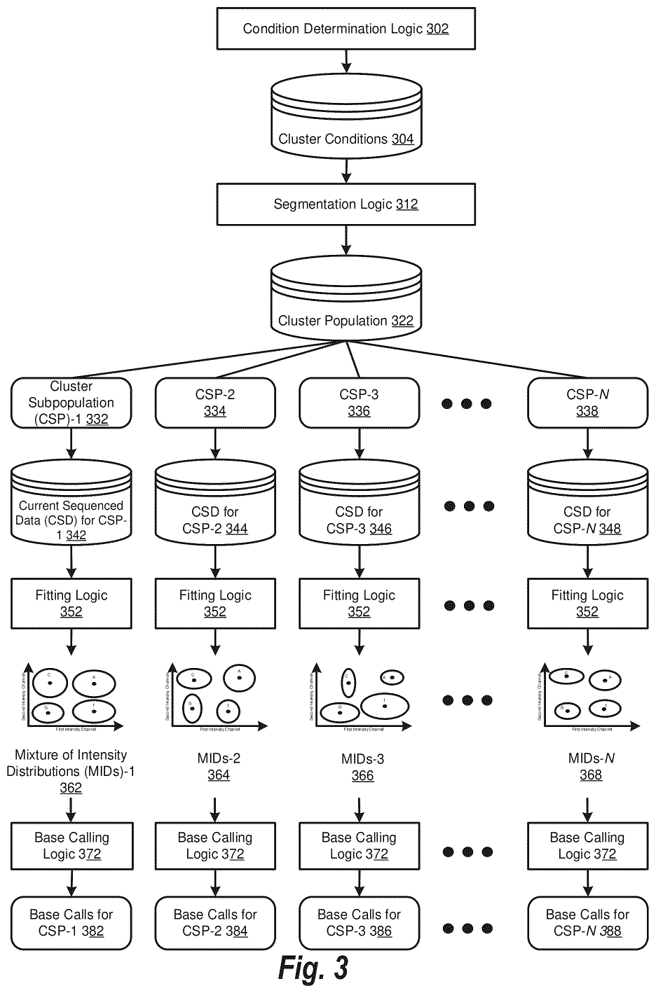

FIG. 3 illustrates an example workflow of segmenting clusters into subpopulations based on segmentation conditions and separately base calling clusters on a subpopulation-by-subpopulation basis. The condition determination logic 302 identifies different cluster segmentation conditions 304 associated with the clusters within a population of clusters 322. The segmentation logic 312 segments, based on the identified segmentation conditions, the population of clusters 322 into a plurality of cluster subpopulations. As illustrated, for example, the plurality of cluster subpopulations includes CSP-1 (332), CSP-2 (334), CSP-3 (336), . . . , CSP-N (338). For a current sequencing cycle, the current sequenced data (e.g., intensity profiles) of the clusters within each subpopulation, namely, CSD for CSP-1 (342), CSD for CSP-2 (344), CSD for CSP-3 (346), . . . , CSD for CSP-N (348) are extracted from sequencing images. A fitting logic 352 fits a mixture of four intensity distributions corresponding to the four bases A, C, T, and G to the current sequenced data for base calling. Since the population of clusters 322 is segmented into a plurality of subpopulations, each cluster subpopulation CSP-1 (332), CSP-2 (334), CSP-3 (336), . . . , CSP-N (338), has a corresponding mixture of intensity distributions MIDs-1 (362), MIDs-2 (364), MIDs-3 (366), . . . , MIDs-N (368), respectively. Each corresponding mixture of intensity distributions represents the clusters having the similar or same segmentation conditions, separated from other subpopulations.

Base calling can be performed by fitting a mathematical model to the intensity profiles of the clusters to be base called. As illustrated in FIG. 3, for a target cluster within a given subpopulation to be base called at a current sequencing cycle, a fitting logic 352 fits a corresponding mixture of four intensity distributions to the intensity values of the target cluster and determines the likelihoods of the intensity values of the target cluster belonging to each of the four intensity distributions.

In some implementations, the mixture of intensity distribution MID is a Gaussian mixture model. A Gaussian mixture model comprises multiple Gaussians, each identified by k ∈ {1, . . . , K}, where K is the number of clustering (i.e., grouping of data points). For example, the Gaussian mixture model can include four intensity distributions, corresponding to four nucleotide bases A, G, C and T. Each Gaussian k in the mixture includes the following parameters:

A mean value μ that defines its centroid.

Covariances Σ that define its width. In a multivariate scenario where, e.g., the intensity profiles for the clusters are extracted from the sequencing images acquired from two color/intensity channels, the covariances Σ define the dimensions of an ellipsoid of the intensity distribution.

In some implementations, the intensity profiles of all clusters within a subpopulation during each sequencing cycle are used for generating the corresponding mixture of intensity distributions. In other implementations, the clusters within the subpopulations are sampled and the intensity profiles of the sampled clusters are used for generating the corresponding mixture of intensity distributions. In yet some other implementations, the sampled clusters within the subpopulation are different at different sequencing cycles. For example, the sampled clusters within a subpopulation for generating a corresponding mixture of intensity distribution at a current sequencing cycle may be different from the sampled clusters at a succeeding sequencing cycle.

In some implementations, for the cluster subpopulations CSP-1, CSP-2, . . . , CSP-N, the fitting and base calling can be performed sequentially to save computation power. In other implementations, for the sake of efficiencies, the fitting and base calling can be performed in parallel.

The parameters of the mixtures of intensity distributions can be iteratively updated. In some implementations, the parameters of the mixtures of intensity distributions can be updated during successive sequencing cycles. For example, the parameters of the mixtures of intensity distributions can be updated at every sequencing cycle during a sequencing run. Alternatively, the parameters of the mixtures of intensity distributions can be updated during non-successive sequencing cycles, for example, alternative sequencing cycles. The parameters of the mixtures of intensity distributions can be updated for a block of sequencing cycles. For example, the parameters of the mixtures of intensity distributions can be updated during each of the five successive sequencing cycles 1-5, 11-15, 21-15 and so on.

In some implementations, the fitting logic 352 includes an expectation maximization algorithm to fit a mixture of intensity distributions to the intensity profiles of the target cluster during a current sequencing cycle. For example, the mixture of intensity distributions is a Gaussian mixture model. Accordingly, the expectation maximization algorithm iteratively maximizes the likelihood of observing means μ (centroids) and covariances Σ (dimensions of the ellipsoid) that best fit the intensity profiles for the target cluster to be base called. For each of the four intensity distributions corresponding to one of the four bases A, C, T, and G, a centroid and covariances of the distribution are calculated. The centroid of the intensity distribution with a maximum likelihood to which the target cluster belong is determined by the base calling logic 372 as the base call for the target cluster.

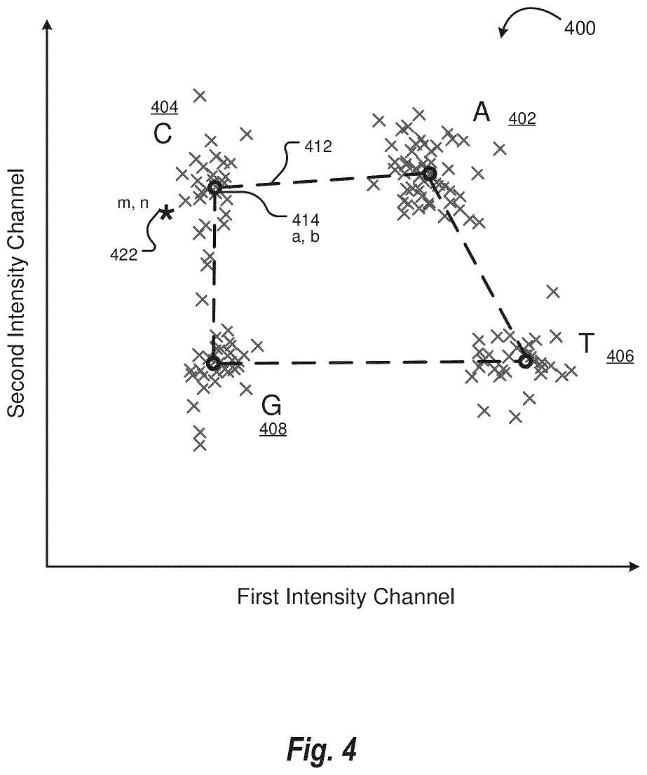

FIG. 4 illustrates an example 400 of how a mixture of intensity distributions fits the intensity profiles of a target cluster for base calling at a current sequencing cycle. The “ [symbol] ” symbol represents the intensity profiles of all clusters within a cluster subpopulation CSP-N at the current sequencing cycle. The four intensity distributions 402, 404, 406 and 408 represent one of the four bases A, C, T and G, respectively. The four intensity distributions take a trapezoid shape 412. The “*” symbol represents the current intensity values “m” and “n” of a target cluster 422 extracted from sequencing images acquired at the first and the second color/intensity channel, respectively. The mixture of the four intensity distributions is fitted to the current intensity values “m” and “n” of the target cluster 422. The mean intensity values “a” and “b” at the centroid 414 of the intensity distribution corresponding to base C at the first and the second intensity channel, respectively. The intensity distribution 404 has a maximum likelihood to which the target cluster belong. Therefore, the target cluster is called as base C at the current sequencing cycle.

In other implementations, other algorithms for grouping datapoints can be used to generate intensity distributions for the four nucleotide bases A, G, C and T, including k-means clustering algorithm, mean-shift clustering algorithm, density-based spatial clustering of applications with noise (DBSCAN), agglomerative hierarchical clustering algorithm. The fitting logic can include a k-means clustering algorithm, a k-means-like clustering algorithm, a histogram-based method, and the like.

Segmenting a population of clusters into subpopulations by segmentation conditions provides various advantages. Sequencing-by-synthesis is a multi-step process, involving sample preparation, sequencing input library generation, cluster formation via amplification, sequencing by incorporating bases into the clusters, etc. Various factors during these steps prior to the sequencing process may bring variations in the properties of clusters which in turn cause variations in the corresponding intensity profiles. These factors can include types of input library types, insert lengths, etc. Other factors during the sequencing process, for example, prior base calls at prior sequencing cycles may also bring variations in the corresponding intensity profiles captured during current sequencing cycle. These factors can include prior base context, signal-to-noise ratio profiles, inter-cluster intensity correction coefficients, signal variation types, etc. Segmenting clusters based on particular segmentation conditions or combinations of conditions ensures clusters with similar to identical conditions are grouped in the same subpopulation. The variations among clusters within the same subpopulation is therefore minimized. During the fitting and base calling processes, the intensity profiles of the clusters within each subpopulation can be well fitted to four intensity distributions corresponding to the four bases A, C, T, and G and to base call target clusters. In other words, each subpopulation of clusters has a corresponding mixture of intensity distributions for base calling, without involving other clusters with different conditions which may bring substantial variations into the subpopulation. As a result, instead of generating intensity distributions using an entire population of clusters, the clusters are separately fitted and base called on a subpopulation-by-subpopulation basis. It minimizes the inter-cluster intensity profile variations and increases the accuracy rate for base calling.

### Condition Determination Logic

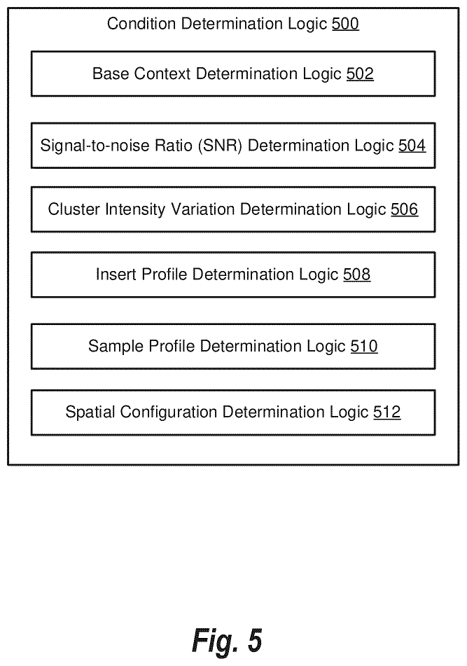

FIG. 5 illustrates various examples of condition determination logic 500 that determine the segmentation conditions for a population of clusters 322. The condition determination logic 500 includes base context determination logic 502 that identifies the base context of clusters. The base context refers to the prior and succeeding bases that are identified at prior and succeeding sequencing cycles, respectively. Analysis has revealed that the intensity profiles of a target cluster at a current sequencing cycle can be shifted based on its base context identified at other sequencing cycles. Therefore, the base context determination logic 502 determines different base context and based on which, those clusters with similar to identical base context are attributed to the same subpopulation.

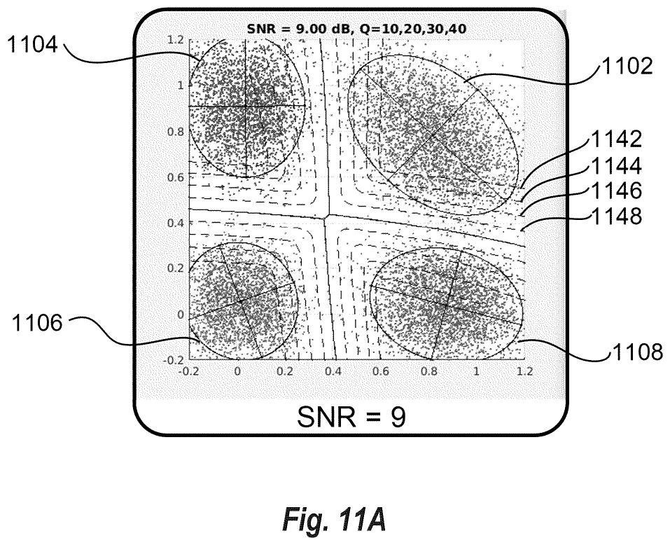

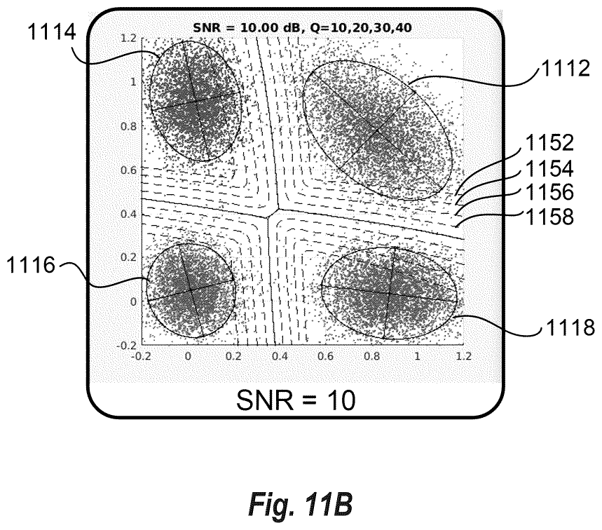

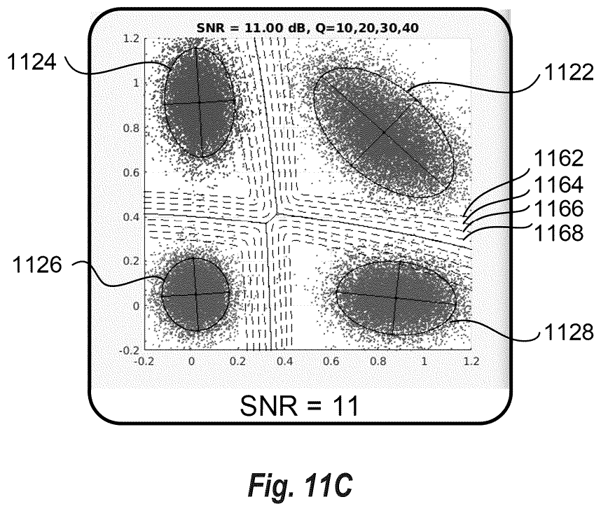

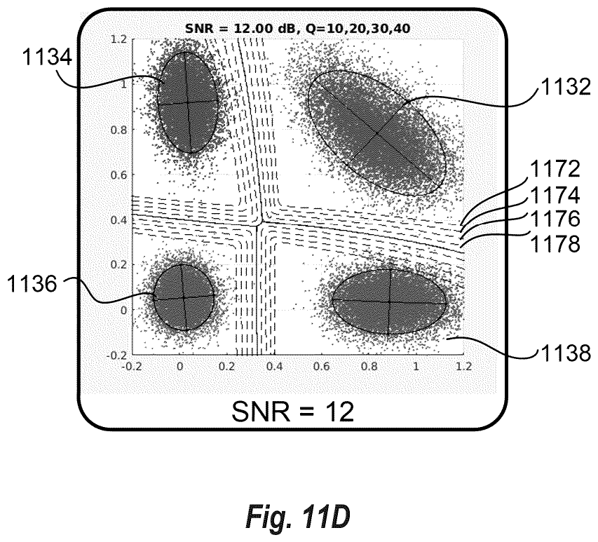

The condition determination logic 500 further includes a signal-to-noise ratio determination logic 504 that identifies signal-to-noise (SNR) ratio profiles of the population of clusters 322. The signal-to-noise ratio determination logic 504 can identify a p number of the different signal-to-noise ratio profiles and based on which, the segmentation logic 312 segments the population of clusters 322 into p subpopulations. The segmentation based on the signal-to-noise (SNR) ratio profiles of the population of clusters will be described in detail in accordance with FIGS. 11A-11D.

The condition determination logic 500 further includes cluster intensity variation determination logic 506. The cluster intensity variation determination logic 506 can identify a v number of different inter-cluster intensity profile variation correction coefficients, and the segmentation logic 312 segments the population of clusters into v subpopulations based on different inter-cluster intensity profile variation correction coefficients.

The condition determination logic 500 further includes an insert profile determination logic 508 and a sample profile determination logic 510. The insert profile determination logic 508 identifies one or more of library types from which clusters are sourced and insert type. The sample profile determination logic 510 identifies sample types and properties of the samples, both of which can be related to the types of input libraries from which clusters are sourced. The segmentation logic 312 segments the population of clusters into subpopulations based on different insert profiles and/or sample profiles of the clusters.

The condition determination logic 500 further includes a spatial configuration determination logic 512. The spatial configuration determination logic 512 identifies the spatial configurations of clusters on a flow cell or a biosensor, including tile locations, sub-tile locations, surface locations, section locations, lane locations, lane group locations, swath locations, and/or swath group locations. The spatial configuration determination logic 512 can identify different locations of clusters and the segmentation logic 312 segments the population of clusters into subpopulations based on different locations of the clusters.

Beginning from segmentation conditions based on base context, next we will describe in detail each condition for cluster segmentation followed by base calling.

### Segmentation Condition Based on Base Context

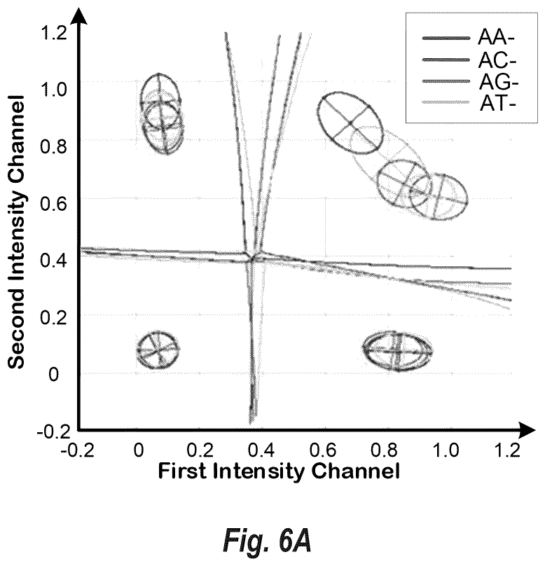

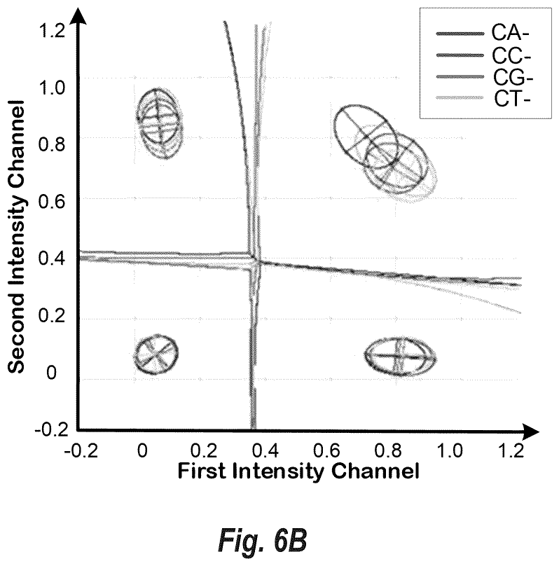

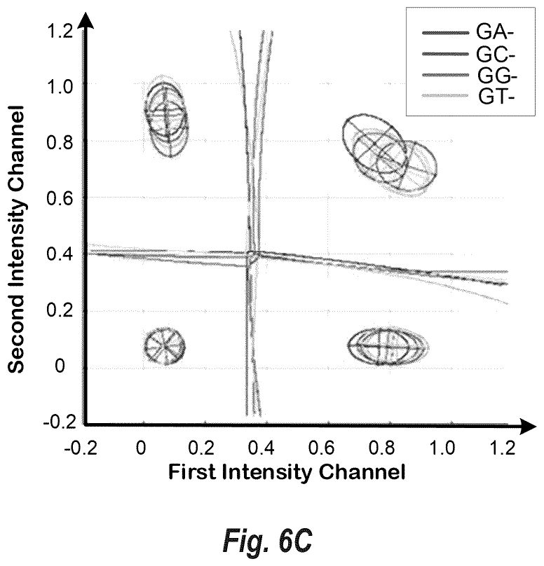

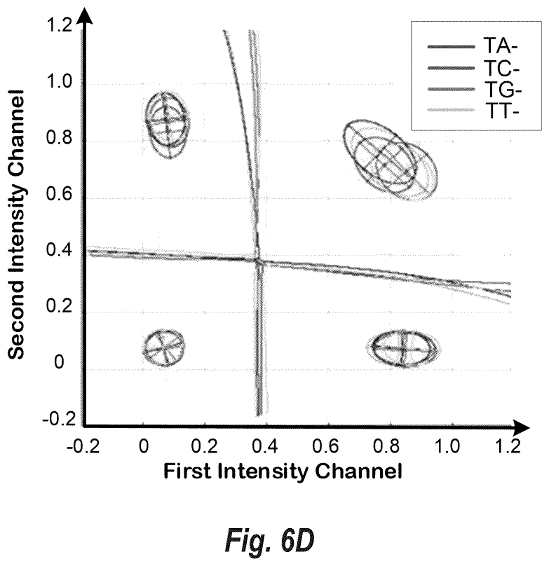

FIGS. 6A-6D illustrate examples of variations caused by prior base context in the intensity distributions of clusters. In particular, FIG. 6A represents the four intensity distributions corresponding to the four bases A, C, T, and G with two prior base context AA (shown in blue), AC (shown in red), AG (shown in green) and AT (shown in yellow). Considering as an example all the clusters to be called as base A, which is the intensity distribution illustrated at the upper right part of FIG. 6A. The different prior base context (e.g., AA, AC, AG and AT) causes a significant shift in the intensity distribution of the clusters to be called. When more prior bases (e.g., three or more prior bases) are taken into consideration, the changes in the intensity distributions can be more significant.

Similarly, FIG. 6B represents the four intensity distributions corresponding to the four bases A, C, T, and G with two prior base context CA (shown in blue), CC (shown in red), CG (shown in green) and CT (shown in yellow). FIG. 6C represents the four intensity distributions corresponding to the four bases A, C, T, and G with two prior base context GA (shown in blue), GC (shown in red), GG (shown in green) and GT (shown in yellow). FIG. 6D represents the four intensity distributions corresponding to the four bases A, C, T, and G with two prior base context TA (shown in blue), TC (shown in red), TG (shown in green) and TT (shown in yellow). When prior base context includes one or more base A, the shift in the intensity distribution can be substantial compared to other bases. These variations in the intensity distributions caused by base context may cause miscalls, especially when an intensity profile of a target cluster to be base called is close to a decision boundary, i.e., between two intensity distributions of different bases, for example, base A and base C, base A and base T.

It should be noted that FIGS. 6A-6D illustrate examples of identical prior bases for the sake of simplicity. A person skilled in the art will appreciate that two prior bases include sixteen different combinations of bases, i.e., AA, AG, AC, AT, CA, CG, CC, CT, GA, GG, GC, GT, TA, TG, TC and TT. Similarly, three prior bases include sixty-four combinations of bases. When k prior bases are included in the base context of a target cluster, there exist 4k combinations. Each combination may cause a particular variation in the intensity distributions which further increases the inter-cluster intensity profile variations.

The base context determination logic 502 determines the base context of clusters such that the segmentation logic 312 segments the population of clusters 322 into subpopulations based on their base context. In one implementation, the base context determination logic determines prior base call segmentation condition, including a single prior base call (A, C, G and T), two prior base calls (e.g., AA, AG, AC, AT, GA . . . ), three prior base calls (e.g., AAA, AAG, AAC, AAT, AGA . . . ) and so on. The prior base calls can be identified at prior sequencing cycles that contiguously precede the current sequencing cycle, and thus, the prior base calls are contiguously preceding base calls. In other implementations, the prior base calls can be identified during prior sequencing cycles that non-contiguously precede the current sequencing cycle, and thus, the prior base calls are non-contiguously preceding base calls.

During SBB, sometimes, the electrons of the fluorophore are transferred to the orbital of pyrimidine bases (thymine (T) and cytosine (C)), or that the electron orbitals of the fluorophore are occupied by electrons from purine bases (guanine (G) and adenine (A)), which lead to so-called “fluorescence quenching.” In addition, the electrons of a fluorophore excited by light can be transmitted along double-stranded DNA, which gives rise to stronger fluorescence quenching.

As an example, the base context determination logic 502 can determine whether the single prior base call immediately preceding the base to be called at the current sequencing cycle is base G. The segmentation logic 312 can segment the population of clusters 322 into two subpopulations, namely, the clusters that with base G called at an immediately preceding sequencing cycle and the clusters that have non-G bases (e.g., A, C, T) called at the immediately preceding sequencing cycle. In a sequencing-by-synthesis (SBS) process, nucleotides that are incorporated into the oligonucleotide strands contained fluorophores that specifically identify the types of the bases and attached to the nucleotides a cleavable linker. After the incorporated base is identified, the linker can be cleaved, allowing the fluorophore to be removed and ready for the next base to be attached and identified. Nevertheless, the cleavage leaves a remaining “pendant arm” moiety located on each of the detected nucleotides, which may impact the intensity profiles of the following nucleotides that are incorporated into the oligonucleotide strands. For example, the remaining “pendant arm” after the cleavage of the fluorophores attached to base G may reduce (or quench) the intensity values of the subsequent fluorophores that are to be attached. When base A with corresponding fluorophores is subsequent to base G, the intensity values of the corresponding fluorophores can be significantly reduced. In a two-channel base calling system where intensity profiles of each base are extracted from two color/intensity channels, for instance, the intensity values of base A following base G at both channels can be reduced. The intensity profiles of other bases (e.g., C and T) can be similarly impacted by the “pendant arm” of the fluorophores attached to base G. By identifying different intensity conditions caused by prior base calls and segmenting the population of clusters into subpopulations, the clusters within each subpopulation can be base called on a subpopulation-by-subpopulation basis. In some implementations, the base context determination logic 502 determines subsequent base call context of the population of clusters 322. The segmentation logic 312 segments these clusters into subpopulations based on their succeeding base call context. The subsequent base calls can be identified at subsequent sequencing cycles that contiguously succeed the current sequencing cycle. Accordingly, the subsequent base calls are contiguously succeeding base calls. In other implementations, the subsequent base calls are identified at subsequent sequencing cycles that non-contiguously succeed the current sequencing cycle. Accordingly, the subsequent base calls are non-contiguously subsequent base calls.

In other implementations, the base context determination logic 502 determines right and left flanking base calls at the right or left flanking sequencing cycles. The segmentation logic 312 segments the population of clusters 322 into subpopulations based on the right and left flanking base calls at the right or left flanking sequencing cycles. For example, the segmentation logic 312 segments the population of clusters 322 into 4(r+1) subpopulations of clusters, where r is a number of succeeding bases called at r succeeding sequencing cycles of a sequencing run, and 1 is a number of prior bases called at I prior sequencing cycles of the sequencing run.

Consider as an example a population of cluster that has been base called for three successive sequencing cycles, namely, cycles n−1, n and n+1. During each of the successive sequencing cycles, the intensity profiles of the clusters are extracted from sequencing images captured from two color/intensity channels. Each of the clusters, based on the corresponding intensity profiles, can have a preliminary base call during each of the three successive sequencing cycles. The segmentation logic can segment the population of target clusters, based on the preliminary base calls identified at left and right flanking sequencing cycles, namely, cycles n−1 and n+1, into 16 subpopulations. Moreover, the intensity profiles of the clusters extracted at left and right sequencing cycle n−1 and sequencing cycle n+1 can be used to correct the intensity profiles extracted at sequencing cycle n, which in turn is used to generate a final base call for sequencing cycle n.

### Segmentation Condition Based on Signal-to-Noise Ratio

As illustrated in FIG. 5, the condition determination logic 500 further includes a signal-to-noise ratio determination logic 504 that identifies signal-to-noise (SNR) ratio profiles of the population of clusters 322. The signal-to-noise ratio determination logic 504 can identify a p number of the different signal-to-noise ratio profiles and based on which, the segmentation logic segments the population of clusters 322 into p subpopulations. The SNR ratio can be calculated as mean called intensity divided by standard deviation of non-called intensities. The mean called intensity refers to the intensity profiles of a target cluster that is base called, where the intensity profiles are extracted from sequencing images captured at a particular color/intensity channel at a particular sequencing cycle of a sequencing run. The non-called intensities refer to the background intensities surrounding the target cluster. Considering the enormous number of clusters immobilized on a flow cell where the clusters vary in sizes, shapes, raw intensities, signal variations, etc., the SNR ratio profile of each cluster can accurately represent the reliability and sensibility of the intensity profiles extracted from the sequencing images during each sequencing cycle. On the other hand, different SNR ratio profiles represent the variations in the intensity profiles among clusters. A large range of SNR ratio profiles may reflect significant variations in the intensity profiles and therefore an increased risk of miscalls and reduced quality scores, whereas a narrow range of SNR ratios reflect the clusters have relatively consistent intensity profiles.

Segmenting clusters conditioned by different SNR ratio profiles can ensure those clusters with similar SNR ratio profiles are attributed to the same subpopulation and thus achieve a good fitting with the intensity distributions for base calling and produce correctly-scaled quality scores. Additionally, SNR ratio profiles take the statistics of undesired signal variations (e.g., noise) into consideration, compared to normalizing the intensity profiles prior to fitting a mixture of intensity distributions. When intensity values are normalized, for example, the 5th and 95th percentile of the intensities have the value of zero and one, respectively, background information are neglected. To the contrary, SNR ratio profiles provide an accurate representation of measured intensity values and background information.

FIGS. 11A-11D illustrate example mixtures of intensity distributions of clusters with different SNR ratios. FIG. 11A depicts a mixture of intensity distributions corresponding to those clusters with the SNR ratio profiles of their intensity values are nine, each of the intensity distributions 1102, 1104, 1106 and 1108 corresponding to one of the four bases A, C, G and T, respectively. FIG. 11B depicts a mixture of intensity distributions corresponding to those clusters with the SNR ratio profiles of their intensity values are ten, the intensity distributions 1112, 1114, 1116 and 1118 corresponding to one of the four bases A, C, G and T, respectively. FIG. 11C depicts a mixture of intensity distributions corresponding those clusters with the SNR ratio profiles of their intensity values are eleven, each of the intensity distributions 1122, 1124, 1126 and 1128 corresponding to one of the four bases A, C, G and T, respectively. FIG. 11D depicts a mixture of intensity distributions corresponding clusters with the SNR ratio profiles of their intensity values are twelve, each of the intensity distributions 1132, 1134, 1136 and 1138 corresponding to one of the four bases A, C, G and T, respectively.

As illustrated in FIGS. 11A-11D, the clusters with different SNR ratio profiles are segmented into subpopulations and for each subpopulation, the parameters (e.g., centroids and covariances) of the mixtures of intensity profiles are different from one another. When the SNR ratio is low (e.g., SNR=9 as illustrated in FIG. 11A), the data points representing the intensity profiles of the clusters are scattered and some of them are close to decision boundaries between two bases. Thus, the error rate of base calling these clusters can be high. When the SNR ratio is high (e.g., SNR=12 as illustrated in FIG. 6D), the data points representing the intensity profiles of the clusters are well distributed, with few of them close to decision boundaries between two bases.

Segmenting clusters based on different SNR ratio profiles also produces correctly-scaled quality scores reflecting the accuracy of base calling. A quality score is a measure of the probability of a sequencing error in a base call. A high quality score implies that a base call is more reliable and less likely to be incorrect. The dashed contour lines 1142 to 1148 in FIG. 11A, 1152 to 1158 in FIG. 11B, 1162 to 1168 in FIG. 11C and 1172 to 1178 in FIG. 11D, represent quality scores Q40, Q30, Q20 and Q10, respectively. When the quality score of a base is Q10, the probability that this base is called incorrectly is 0.1, and the base call accuracy is 90%. Similarly, when the quality score of a base is Q20, the probability that this base is called incorrectly is 0.01, and the base call accuracy is 99%. When the quality score of a base is Q30, the probability that this base is called incorrectly is 0.001, and the base call accuracy is 99.9%. When the quality score of a base is Q40, the probability that this base is called incorrectly is 0.0001, and the base call accuracy is 99.99%.

When the SNR ratio is low (e.g., SNR=9 as illustrated in FIG. 11A), a substantial portion of the datapoints fall between Q10 and Q20. When the SNR ratio is high (e.g., SNR=12 as illustrated in FIG. 6D), most of the data points corresponding to intensity profiles of clusters to be base called have high quality scores, indicating a low probability of sequencing errors in base calling.

### Segmentation Condition Based on Cluster Intensity Variations

As illustrated in FIG. 5, the condition determination logic 500 further includes cluster intensity variation determination logic 506. In some implementations, the cluster intensity variation determination logic 506 identifies a v number of different inter-cluster intensity profile variation correction coefficients, and the segmentation logic segments the population of clusters into v subpopulations based on different inter-cluster intensity profile variation correction coefficients. In one implementation involving a two color/intensity channel sequencing system, the variation correction coefficients include two channel-specific amplification coefficients that account for (or correct) scale variations in the inter-cluster intensity profiles, and two channel-specific offset coefficients that account for (or correct) shift variation along the first and the second intensity channels in the inter-cluster intensity profile variation, respectively. In another implementation, the scale variation can be accounted for by using a common amplification coefficient for different intensity channels. Similarly, the shift variation can also be accounted for by using a common offset coefficient for different intensity channels. To use inter-cluster intensity profile variation correction coefficients as segmentation conditions ensures those clusters with similar correction coefficients (i.e., with similar levels of variations in the corresponding intensity profiles), to be attributed to the same subpopulation for base calling.

For a target cluster, its corresponding variation correction coefficients can be generated at a current sequencing cycle of a sequencing run based on the historic intensity statistics determined for the target cluster at prior sequencing cycles and current intensity statistics determined for the target cluster at the current sequencing cycle. The generated variation correction coefficients can be used to correct next intensity readings registered for the target cluster at a next sequencing cycle succeeding the current sequencing cycle. The corrected next intensity readings are used to base call the target cluster at the next sequencing cycle. This correction process can repeat at each sequencing cycle of the sequencing run. That is, to repeatedly apply respective variation correction coefficients to respective intensity profiles of respective clusters at successive sequencing cycles. As a result, the intensity profiles of the clusters become coincidental and anchored to the origin of the intensity distribution (e.g., origin 210 at the bottom lower corner of the trapezoids as illustrated in FIG. 2). Additional details about the calculation of variation correction coefficients can be found in U.S. Patent Application Publication No. 2022/0129711A1, the disclosure of which is incorporated herein by reference in its entirety.

In other implementations, the cluster intensity variation determination logic 506 identifies different raw intensity profiles and/or corrected intensity profiles of clusters, and the segmentation logic 312 segments clusters based on their intensity profiles. The cluster intensity variation determination logic 506 can identify a j number of different raw intensity profiles for the clusters, and the segmentation logic 312 segments the clusters into j subpopulations based on their different raw intensity profiles. Raw intensity profiles of the clusters can include the intensity values extracted from sequencing images without correction. The raw intensity profiles can be subsequently corrected to generate corrected intensity profiles. In some implementations, the raw intensity profiles can be corrected for spatial crosstalk, which is an interference from adjacent clusters and makes it difficult to distinguish true light signals generated by a cluster of interest from other unwanted light signals from neighboring clusters. In other implementations, the raw intensity profiles can be corrected for phasing and pre-phasing, which also increase signal variations as the sequencing run proceeds. Phasing refers to steps in sequencing in which the tags fail to advance along the sequence. Pre-phasing refers to sequencing steps in which the tags jump two positions forward instead of one, during a sequencing cycle.

The cluster intensity variation determination logic 506 can identify different signal variation types detected in the intensity profiles of the clusters including, for example, crosstalk, phasing and pre-phasing, background signals and signal decay during the sequencing process. The cluster intensity variation determination logic 506 can identify a n number of different signal variation types for the population of clusters, and the segmentation logic 312 segments the clusters into n subpopulations based on different signal variation types.

### Segmentation Condition Based on Insert Profiles and Sample Profiles

As illustrated in FIG. 5, the condition determination logic 500 further includes an insert profile determination logic 508 and a sample profile determination logic 510. The insert profile determination logic 508 determines one or more of library types from which clusters are sourced and insert type. The sample profile determination logic 510 identifies sample types and properties of the samples, both of which can be related to the types of input libraries from which clusters are sourced.

In some implementations, the insert profile determination logic 508 identifies the types of input libraries. The insert profile determination logic 508 can identify a s number of different library types, and the segmentation logic 312 segments a population of clusters into s subpopulations of clusters based on the different library types. An input library is a collection of DNA fragments with similar lengths and connected with known adaptor sequences attached to the 5′ and 3′ ends of the fragments. Different input libraries may have different types of inserts, indexing (first index read v/s second index read), reads (forward read v/s reverse read), and insert lengths. Accordingly, the insert profile determination logic 508 can also identify an i number of different insert lengths, and the segmentation logic 312 segments the population of clusters into i subpopulations of clusters based on different insert lengths.

After nucleic acid (DNA or RNA) is extracted from a biological sample, it is fragmented to a plurality of target fragments with relatively short length, followed by ligating specific adaptor sequences to both ends of each target fragment, to construe a sequencing input library. Various factors, including the quantity and physical characteristics of the source sample material as well as the desired applications (e.g., genome sequencing, targeted sequencing, exome sequencing, RNA-seq, ChIP-seq, RIP-seq, and methylation), influence the input library and the properties of the fragments in the library. Identifying the library types and segmenting the clusters that are sourced from different library types is advantageous when clusters generated from different libraries are immobilized on the same flow cell or biosensor.

The size of sequencing input libraries is also related to insert lengths. Inserts refer to the target fragments between adapter sequences. The length of inserts can be in a range from below 100 bp to 1000 bp. In some implementations, an optimal insert size is determined by the NGS instrumentations and specific sequencing applications. For example, when constructing sequencing libraries to be used in Illumina' sequencer, an optimal insert size is impacted by the process of cluster generation in which libraries are denatured, diluted and distributed on the two-dimensional surface of the flow cell and then amplified. While shorter inserts amplify more efficiently than longer products, longer library inserts generate larger, more diffused clusters. An optimal size of an input library is also dictated by sequencing applications. In exome sequencing, for example, more than 80% of human exomes are under 200 bases in length. In the case of microRNA (miRNA)/small RNA library, the desired insert size is only 20-30 bases larger than the size of the adaptors.

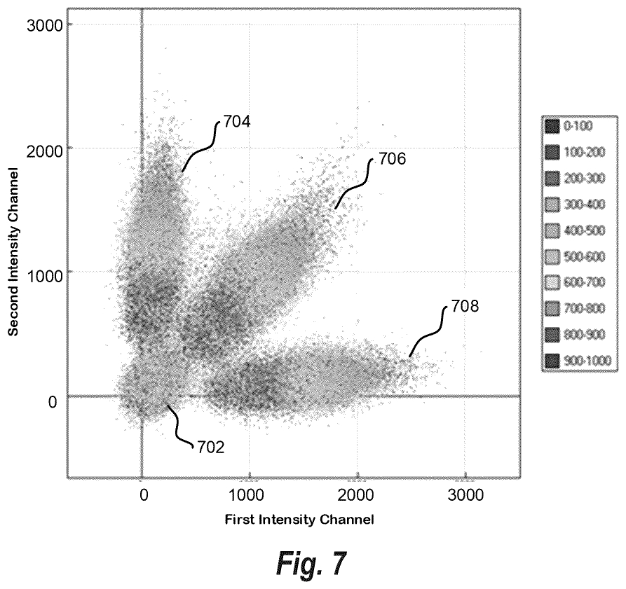

Since a cluster is a colony of oligonucleotides with the identical sequences amplified from the sequencing input library, the lengths of inserts also causes variations in the intensity profiles among clusters. FIG. 7 illustrates the intensity distributions of clusters with different insert lengths. A first nucleotide type (e.g., base T) 708 is detected at the first intensity channel (x-axis of the multi-dimensional space 700). A second nucleotide type (e.g., base C) 704 is detected at the second intensity channel (y-axis of the multi-dimensional space 700). A third nucleotide type (e.g., base A) 706 is detected at both the first and the second intensity channels. And a fourth nucleotide type (e.g., base G) 702 that lacks a label is not, or minimally, detected in either of the intensity channels. As illustrated, the intensity distribution of base G 702 is minimally impacted by the lengths of inserts because the intensities extracted from both intensity channels are minimal. Nevertheless, the intensity distributions of other three bases A, C and T (706, 704 and 708, respectively) are substantially impacted by insert length. The longer the inserts (e.g., 700-800 bp, 800-900 bp and 900-1000bp), the lower are the intensity values. When clusters with similar insert lengths are attributed to the same subpopulation for fitting a corresponding mixture of intensity distributions and base calling, the intensity variations caused by different insert lengths can be minimized.

In some implementations, the sample profile determination logic 510 identifies the types and properties of samples that are used to generate sequencing input libraries. Different types and/properties of samples relate to the types of the input libraries, which in turn cause inter-cluster intensity variations. Thus, it is important to identify and differentiate the types and/properties of samples when preparing input libraries from which clusters are generated. The sample profile determination logic 510 can identify a x number of different sample types, and the segmentation logic 312 segments, based on different sample types, a population of clusters into x subpopulations. Alternatively or additionally, the sample profile determination logic can identify a o number of different physical properties of samples from which the population of clusters is sourced, and the segmentation logic segments the population of clusters into o subpopulations. The samples to be sequenced can include DNA, RNA, PNA, LNA, chimeric or hybrid forms of nucleic acids. The samples can include biological, clinical, surgical, agricultural, atmospheric, or aquatic-based specimen containing one or more nucleic acids. The sample can include isolated nucleic acid sample such as genomic DNA, fresh-frozen or formalin-fixed paraffin-embedded nucleic acid specimen. The samples can be from a single individual, a collection of nucleic acid samples from genetically related members, a collection of nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some implementations, the source of nucleic acid material can include nucleic acids obtained from a newborn, for example as typically used for newborn screening.

The samples can include high molecular weight material such as genomic DNA (gDNA). The samples can include low molecular weight material such as nucleic acid molecules obtained from formalin-fixed, paraffin-embedded (FFPE) or archived DNA samples. In another implementation, low molecular weight material includes enzymatically or mechanically fragmented DNA. The sample can include cell-free circulating DNA. In some implementations, the samples can include nucleic acid molecules obtained from biopsies, tumors, scrapings, swabs, blood, mucus, urine, plasma, semen, hair, laser capture micro-dissections, surgical resections, and other clinical or laboratory obtained samples. In some implementations, the sample can be an epidemiological, agricultural, forensic, or pathogenic sample. In other implementations, the samples can include nucleic acid molecules obtained from an animal such as a human or mammalian source. In another implementation, the sample can include nucleic acid molecules obtained from a non-mammalian source such as a plant, bacteria, virus, or fungus. In some implementations, the source of the nucleic acid molecules may be an archived or extinct sample or species. The nucleic acid samples can have low-quality nucleic acid molecules, such as degraded and/or fragmented genomic DNA from forensic samples. The forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The sample may be a purified sample or a crude DNA containing lysate, for example derived from a buccal swab, paper, fabric, or other substrate that may be impregnated with saliva, blood, or other bodily fluids. As such, in some implementations, the samples may comprise low amounts of, or fragmented portions of nucleic acid, such as genomic DNA. The target sequences can be present in one or more bodily fluids including but not limited to, blood, sputum, plasma, semen, urine, and serum. In some implementations, target sequences can be obtained from hair, skin, tissue samples, autopsy, or remains of a victim. In some implementations, nucleic acids including one or more target sequences can be obtained from a deceased animal or human. In some implementations, target sequences can include nucleic acids obtained from non-human DNA such a microbial, plant or entomological DNA.

### Segmentation Condition Based on Spatial Configurations of Clusters

As illustrated in FIG. 5, condition determination logic 500 further includes a spatial configuration determination logic 512. The spatial configuration determination logic 512 identifies the spatial configurations of clusters on a flow cell or a biosensor, including tile locations, sub-tile locations, surface locations, section locations, lane locations, lane group locations, swath locations, and/or swath group locations. The spatial configuration determination logic 512 can identify a tile-specific condition, for the clusters located on a particular tile or a particular tile-type/category/class (e.g., central tiles or peripheral tiles or tiles 1 to N of a flow cell. The spatial configuration determination logic 512 can identify a sub-tile-specific condition, for the clusters located on a particular sub-tile or a particular sub-tile-type/category/class (e.g., central sub-tiles or peripheral sub-tiles or sub-tiles 1 to N of a flow cell). The spatial configuration determination logic 512 can identify a surface-specific condition, for the clusters located on a particular surface or a particular surface-type/category/class (e.g., top surfaces or bottom surfaces or surfaces 1 to N of a flow cell). The spatial configuration determination logic 512 may identify a section-specific condition, for the clusters located on a particular section or a particular section-type/category/class. In other implementation, the spatial configuration determination logic 512 can identify a lane-specific condition, for the clusters located on a particular lane or a particular lane-type/category/class (e.g., central lanes or peripheral lanes or lanes 1 to N of a flow cell). When at least two lanes are grouped into a particular lane group (e.g., lane pair), the spatial configuration determination logic 512 can identify a lane group-specific condition, for the clusters located on a particular lane group or a particular lane group-type/category/class (e.g., central lane groups or peripheral lane groups or lane groups 1 to N of a flow cell). The spatial configuration determination logic 512 can identify a swath-specific condition, for the clusters located on a particular swath or a particular swath-type/category/class (e.g., central swath or peripheral swath or swaths 1 to N of a flow cell). A swath refers to a column of tiles in one lane, and there are two swaths per lane surface. When at least two swaths are grouped into a swath group, the spatial configuration determination logic 512 can identify a swath group-specific condition, for the clusters located on a particular swath group or a particular swath group-type/category/class (e.g., central swath groups or peripheral swath groups or swath groups 1 to N of a flow cell).

Without limiting the scope of the disclosure, other examples of segmentation conditions that the condition determination logic 302/500 identifies include imaging types, color channel types, laser types, optics types, lens types, optical filter types, illumination types, indexing types, read types, reagent types, etc. In one implementation, the condition determination logic 302/500 can identify the segmentation conditions by index reads, including single-indexing, dual-indexing, unique dual-indexing, combinatorial dual-indexing, etc. The condition determination logic 302/500 can identify a y number of different index reads in a population of clusters, and the segmentation logic 312 segments the clusters into y subpopulations based on different index reads. In another implementation, the condition determination logic 302/500 can identify the cluster conditions by read types, including paired-end sequencing, single-read sequencing, forward read, reverse read, etc. The condition determination logic 302/500 can identify a z number of different read types for the population of clusters, and the segmentation logic 312 segments the clusters into z. subpopulations based on the different read types. In other implementations, the condition determination logic 302/500 can identify a m number of different reagent types used for a population of clusters, and the segmentation logic 312 segments the clusters into m subpopulations based on the different reagent types.

One skilled in the art would also appreciate the condition determination logic 302/500 can identify a plurality of segmentation conditions and the segmentation logic 312 can segment, based on the plurality of segmentation conditions, a population of clusters into subpopulations. In some implementations, the condition determination logic 302/500 can identify three prior bases with sixty-four combinations of bases, as well as lane-specific spatial configurations of the target clusters immobilized on a flow cell including eight lanes. Accordingly, the condition determination logic 500 can determine 64×8 segmentation conditions.

### Data Segmentation Logic

As illustrated in FIG. 3, the segmentation logic 312 segments a population of clusters 322 into a plurality of cluster subpopulations based on one or more segmentation conditions identified by the condition determination logic 302. Each cluster subpopulation includes a plurality of clusters having the same segmentation condition or combinations of segmentation condition. For a target cluster within a given subpopulation CSP-N, the fitting logic 352 iteratively fits a mixture of intensity distributions MIDs-1 (362) corresponding to the given subpopulation to the intensity values of the target cluster CSD-N at current sequencing cycle. The base calling logic 372 determines the intensity distribution to which the target cluster belong with a maximum likelihood and identifies the base call CSP-N for the target cluster, such as by determining base calls for CSP-1 (382), base calls for CSP-2 (384), base calls for CSP-3 (386), . . . , base calls for CSP-N (388).

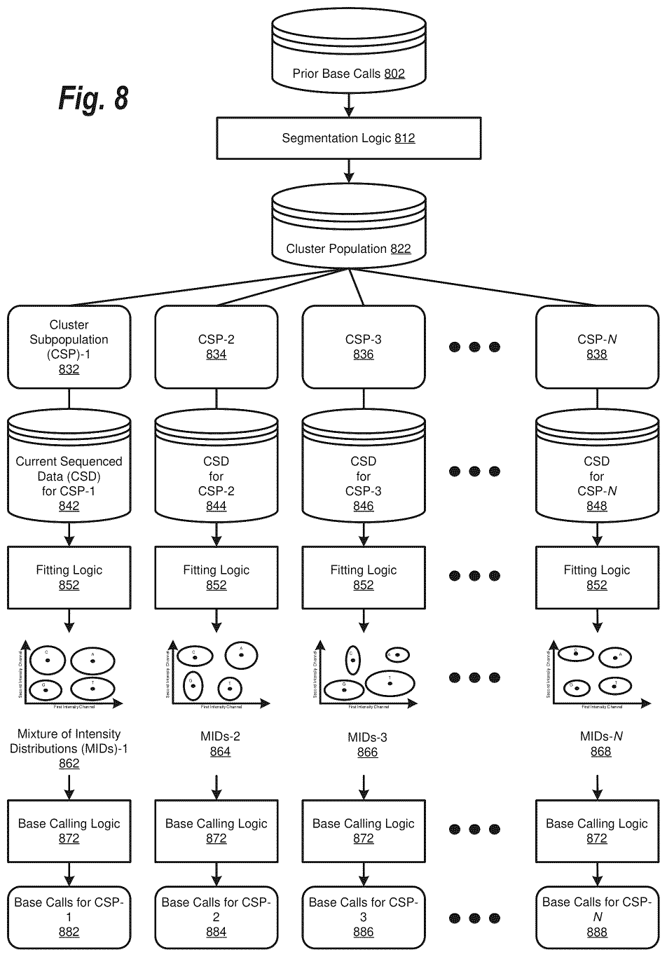

FIG. 8 illustrates another example workflow of segmenting a population of clusters into subpopulations based on segmentation conditions and separately base calling clusters on a subpopulation-by-subpopulation basis. Based on the segmentation conditions of prior base calls 802, the segmentation logic 812 segments a population of clusters 822 into a plurality of cluster subpopulations CSP-1 (832), CSP-2 (834), CSP-3 (836) . . . , CSP-N (838). Different from FIG. 3 where the condition determination logic 302 and the segmentation logic 312 identifies cluster conditions and segments clusters into subpopulations, FIG. 8 illustrates the clusters are segmented into subpopulations based on their prior base calls 802. The prior base calls 802 can include prior base call context, referring to base calls determined at prior sequencing cycles. The prior base calls 802 can also include signals (e.g., intensity values extracted from different color/intensity channel) of the clusters that are base called at prior sequencing cycles. For example, the clusters that are base called at prior sequencing cycles have different signal-to-noise ratio (SNR) profiles. These clusters can be segmented into subpopulations by the segmentation logic 812 based on their SNR profiles.

Similar to FIG. 3, for a target cluster within a given subpopulation CSP-N, the fitting logic 852 in FIG. 8 iteratively fits a mixture of intensity distributions MIDs-1 corresponding to the given subpopulation to the intensity values of the target cluster CSD-N at current sequencing cycle, namely, by fitting MIDs-1 (862) to CSD for CSP-1 (842), fitting MIDs-1 (864) to CSD for CSP-2 (844), fitting MIDs-1 (866) to CSD for CSP-3 (846), . . . , fitting MIDs-1 (868) to CSD for CSP-N (848). Also similar to FIG. 3, the base calling logic 872 in FIG. 8 determines the intensity distribution to which the target cluster belong with a maximum likelihood and identifies the base call CSP-N for the target cluster, such as by determining base calls for CSP-1 (882), base calls for CSP-2 (884), base calls for CSP-3 (886), . . . , base calls for CSP-N (888).

### Segmentation of Clusters Based on Base Call Context

The segmentation conditions of prior base calls can include prior base call context. As shown in FIGS. 6A-6D, prior base call context can significantly impact the intensity distributions of clusters. The numbers of prior base calls at prior sequencing cycles (e.g., one prior base, two prior bases, three prior bases) can also impact the intensity distributions of clusters. Consider as an example those clusters with current base as A at currently sequencing cycle. The segmentation logic 312/812 can segments those clusters into four subpopulations, corresponding to the clusters with two prior base calls of AA, AG, AC and AT, respectively. The two prior base calls are identified at two prior sequencing cycles proceeding the current sequencing cycle. The intensity distributions for those clusters within the four subpopulations are substantially different from one another. In addition, the prior base context also impacts the locations of decision boundaries. A decision boundary is located between the intensity distributions of two different bases, for example, between A and C, A and T, C and G, as well as T and G. It is important to determine an accurate decision boundary in order to reduce the error rate. Still consider the example of those clusters with current base call of A at current sequencing cycle and two prior base calls of AA, AG, AC and AT, respectively, at prior sequencing cycles. Because of the substantial shift in the intensity distributions of the clusters in the four subpopulations, the corresponding decision boundaries between bases A and C as well as A and T are also shifted. By segmenting clusters by segmentation conditions, clusters within each subpopulation can be independently processed to generate corresponding intensity distributions for base calling the clusters therein, the intensity distributions and decision boundaries are accurately determined, thereby minimizing the variances caused by clusters with different conditions.

In some implementations, the segmentation logic 312/812 segments the population of clusters 822 into 4 k subpopulations of clusters based on k prior bases called at k prior sequencing cycles of the sequencing run (k=1, 2, 3, 4 . . . ). For example, when the segmentation is based on a single prior base called at a prior sequencing cycle of the sequencing run, the segmentation logic segments the population of clusters into four subpopulations of clusters. The four subpopulations include a first subpopulation including those clusters that had an A base call at the prior sequencing cycle; a second subpopulation including those clusters that had a C base call at the prior sequencing cycle; a third subpopulation including those clusters that had a G base call at the prior sequencing cycle; and a fourth subpopulation including those clusters that had a T base call at the prior sequencing cycle. The intensity profiles of the clusters within each of the four subpopulations are fitted to a corresponding mixture of intensity distributions for base calling, independent from other subpopulations.

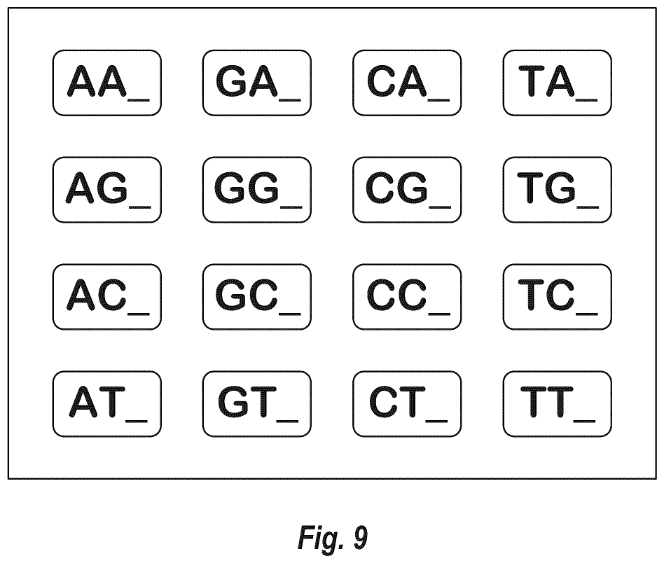

In other implementations, when the segmentation is based on two prior bases called at two prior sequencing cycles of a sequencing run, the segmentation logic 312/812 segments the population of clusters into sixteen subpopulations of clusters. FIG. 9 illustrates an example of sixteen subpopulations based on two prior bases, including a first subpopulation including those clusters that had AA base calls at the two prior sequencing cycles; a second subpopulation including those clusters that had AC base calls at the two prior sequencing cycles; a third subpopulation including those clusters that had AG base calls at the two prior sequencing cycles; a fourth subpopulation including those clusters that had AT base calls at the two prior sequencing cycles; a fifth subpopulation including those clusters that had CA base calls at the two prior sequencing cycles; a sixth subpopulation including those clusters that had CC base calls at the two prior sequencing cycles; a seventh subpopulation including those clusters that had CG base calls at the two prior sequencing cycles; a eighth subpopulation including those clusters that had CT base calls at the two prior sequencing cycles; a ninth subpopulation including those clusters that had GA base calls at the two prior sequencing cycles; a tenth subpopulation including those clusters that had GC base calls at the two prior sequencing cycles; a eleventh subpopulation including those clusters that had GG base calls at the two prior sequencing cycles; a twelfth subpopulation including those clusters that had GT base calls at the two prior sequencing cycles; a thirteenth subpopulation including those clusters that had TA base calls at the two prior sequencing cycles; a fourteenth subpopulation including those clusters that had TC base calls at the two prior sequencing cycles; a fifteenth subpopulation including those clusters that had TG base calls at the two prior sequencing cycles; and a sixteenth subpopulation including those clusters that had TT base calls at the two prior sequencing cycles. The intensity profiles of the clusters within each of the sixteen subpopulations are fitted to a corresponding mixture of intensity distributions for base calling, independent from other subpopulations.

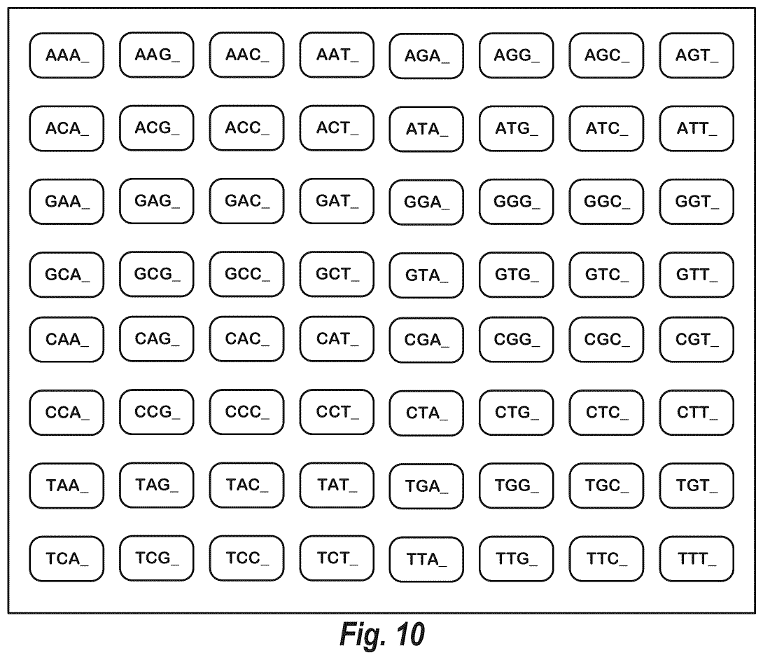

In other implementations, when the segmentation is based on three prior bases called at three prior sequencing cycles of a sequencing run, the segmentation logic 312/812 segments the population of clusters into sixty-four subpopulations of clusters. FIG. 10 illustrates sixty-four subpopulations based on three prior base context, including a first subpopulation including those clusters that had AAA base calls at the three prior sequencing cycles; a second subpopulation including those clusters that had AAC base calls at the three prior sequencing cycles; a third subpopulation including those clusters that had AAG base calls at the three prior sequencing cycles; a fourth subpopulation including those clusters that had AAT base calls at the three prior sequencing cycles; a fifth subpopulation including those clusters that had ACA base calls at the three prior sequencing cycles; a sixth subpopulation including those clusters that had ACC base calls at the three prior sequencing cycles; a seventh subpopulation including those clusters that had ACG base calls at the three prior sequencing cycles; a eighth subpopulation including those clusters that had ACT base calls at the three prior sequencing cycles; a ninth subpopulation including those clusters that had AGA base calls at the three prior sequencing cycles; a tenth subpopulation including those clusters that had AGC base calls at the three prior sequencing cycles; a eleventh subpopulation including those clusters that had AGG base calls at the three prior sequencing cycles; a twelfth subpopulation including those clusters that had AGT base calls at the three prior sequencing cycles; a thirteenth subpopulation including those clusters that had ATA base calls at the three prior sequencing cycles; a fourteenth subpopulation including those clusters that had ATC base calls at the three prior sequencing cycles; a fifteenth subpopulation including those clusters that had ATG base calls at the three prior sequencing cycles; a sixteenth subpopulation including those clusters that had ATT base calls at the three prior sequencing cycles; a seventeenth subpopulation including those clusters that had CAA base calls at the three prior sequencing cycles; an eighteenth subpopulation including those clusters that had CAC base calls at the three prior sequencing cycles; a nineteen subpopulation including those clusters that had CAG base calls at the three prior sequencing cycles; a twentieth subpopulation including those clusters that had CAT base calls at the three prior sequencing cycles; a twenty-first subpopulation including those clusters that had CCA base calls at the three prior sequencing cycles; a twenty-second subpopulation including those clusters that had CCC base calls at the three prior sequencing cycles; a twenty-third subpopulation including those clusters that had CCG base calls at the three prior sequencing cycles; a twenty-fourth subpopulation including those clusters that had CCT base calls at the three prior sequencing cycles; a twenty-fifth subpopulation including those clusters that had CGA base calls at the three prior sequencing cycles; a twenty-sixth subpopulation including those clusters that had CGC base calls at the three prior sequencing cycles; a twenty-seventh subpopulation including those clusters that had CGG base calls at the three prior sequencing cycles; a twenty-eighth subpopulation including those clusters that had CGT base calls at the three prior sequencing cycles; a twenty-nineth subpopulation including those clusters that had CTA base calls at the three prior sequencing cycles; a thirtieth subpopulation including those clusters that had CTC base calls at the three prior sequencing cycles; a thirty-first subpopulation including those clusters that had CTG base calls at the three prior sequencing cycles; a thirty-second subpopulation including those clusters that had CTT base calls at the three prior sequencing cycles; a thirty-third subpopulation including those clusters that had GAA base calls at the three prior sequencing cycles; a thirty-fourth subpopulation including those clusters that had GAC base calls at the three prior sequencing cycles; a thirty-fifth subpopulation including those clusters that had GAG base calls at the three prior sequencing cycles; a thirty-sixth subpopulation including those clusters that had GAT base calls at the three prior sequencing cycles; a thirty-seventh subpopulation including those clusters that had GCA base calls at the three prior sequencing cycles; a thirty-eighth subpopulation including those clusters that had GCC base calls at the three prior sequencing cycles; a thirty-nineth subpopulation including those clusters that had GCG base calls at the three prior sequencing cycles; a fortieth subpopulation including those clusters that had GCT base calls at the three prior sequencing cycles; a forty-first subpopulation including those clusters that had GGA base calls at the three prior sequencing cycles; a forty-second subpopulation including those clusters that had GGC base calls at the three prior sequencing cycles; a forty-third subpopulation including those clusters that had GGG base calls at the three prior sequencing cycles; a forty-fourth subpopulation including those clusters that had GGT base calls at the three prior sequencing cycles; a forty-fifth subpopulation including those clusters that had GTA base calls at the three prior sequencing cycles; a forty-sixth subpopulation including those clusters that had GTC base calls at the three prior sequencing cycles; a forty-seventh subpopulation including those clusters that had GTG base calls at the three prior sequencing cycles; a forty-eighth subpopulation including those clusters that had GTT base calls at the three prior sequencing cycles; a forty-nineth subpopulation including those clusters that had TAA base calls at the three prior sequencing cycles; a fiftieth subpopulation including those clusters that had TAC base calls at the three prior sequencing cycles; a fifty-first subpopulation including those clusters that had TAG base calls at the three prior sequencing cycles; a fifty-second subpopulation including those clusters that had TAT base calls at the three prior sequencing cycles; a fifty-third subpopulation including those clusters that had TCA base calls at the three prior sequencing cycles; a fifty-fourth subpopulation including those clusters that had TCC base calls at the three prior sequencing cycles; a fifty-fifth subpopulation including those clusters that had CCG base calls at the three prior sequencing cycles; a fifty-sixth subpopulation including those clusters that had TCT base calls at the three prior sequencing cycles; a fifty-seventh subpopulation including those clusters that had TGA base calls at the three prior sequencing cycles; a fifty-eighth subpopulation including those clusters that had TGC base calls at the three prior sequencing cycles; a fifty-nineth subpopulation including those clusters that had TGG base calls at the three prior sequencing cycles; a sixtieth subpopulation including those clusters that had TGT base calls at the three prior sequencing cycles; a sixty-first subpopulation including those clusters that had TTA base calls at the three prior sequencing cycles; a sixty-second subpopulation including those clusters that had TTC base calls at the three prior sequencing cycles; a sixty-third subpopulation including those clusters that had TTG base calls at the three prior sequencing cycles; a sixty-fourth subpopulation including those clusters that had TTT base calls at the three prior sequencing cycles. The intensity profiles of the clusters within each of the sixty-four subpopulations are fitted to a corresponding mixture of intensity distributions for base calling, independent from other subpopulations.

The prior base calls can be identified during prior sequencing cycles that are contiguously prior to current sequencing cycle. Accordingly, the prior base calls are contiguously prior base calls. Alternatively or additionally, the prior base calls are identified during the prior sequencing cycles that are non-contiguously prior to the current sequencing cycle. Accordingly, the prior base calls are non-contiguously prior base calls.

The base call context information can include succeeding base calls. In some implementations, the segmentation logic 312/812 segments the population of clusters 822 into the plurality of subpopulations based on succeeding base calls at subsequent sequencing cycles of a sequencing run. The succeeding base calls can be identified at subsequent sequencing cycles that are contiguously succeeding the current sequencing cycle. Accordingly, the succeeding base calls are contiguously succeeding base calls. Alternatively or additionally, the succeeding base calls are identified at subsequent sequencing cycles that are non-contiguously succeeding the current sequencing cycle. Accordingly, the succeeding base calls are non-contiguously succeeding base calls.

The base call context information can include right and left flanking base calls at the right or left flanking sequencing cycles of a sequencing run. For example, the segmentation logic segments the population of clusters into 4(r+1) subpopulations of clusters, where r is a number of succeeding bases called at r succeeding sequencing cycles of the sequencing run, and 1 is a number of prior bases called at 1 prior sequencing cycles of the sequencing run.

Consider as an example a population of cluster that has been base called for three successive sequencing cycles, namely, cycles n−1, n and n+1. During each of the successive sequencing cycles, the intensity profiles of the clusters are extracted from sequencing images captured from two color/intensity channels. Each of the clusters, based on the corresponding intensity profiles, can have a preliminary base call during each of the three successive sequencing cycles. The segmentation logic 312/812 can segment the population of clusters, based on the preliminary base calls identified at left and right flanking sequencing cycles, namely, cycles n−1 and n+1, into 16 subpopulations. Moreover, the intensity profiles of the clusters extracted at left and right sequencing cycle n−1 and sequencing cycle n+1 can be used to correct the intensity profiles extracted at sequencing cycle n, which in turn is used to generate a final base call for sequencing cycle n.

### Segmentation of Clusters Based on Signal-to-Noise Ratio

The segmentation logic 312/812 can segment the population of clusters 822 into a plurality of subpopulations based on different SNR ratio profiles (e.g., SNR ratio ranges) of the intensity values of the clusters. As illustrated in FIGS. 11A-11D, for a given sequencing cycle, each cluster within the population has a corresponding SNR ratio, determined by the SNR determination logic 504. The segmentation logic 312/812 segments the population of clusters into four subpopulations, corresponding to SNR=9, SNR=10, SNR=11 and SNR=12, respectively. Each subpopulation of clusters has a corresponding mixture of intensity distribution with regard to four bases A, G, C and T.

In some implementations, the SNR determination logic 504 can compute and store SNR ratio profiles for each cluster at each sequencing cycle of a sequencing run. Accordingly, at each sequencing cycles, the segmentation logic 312/812 attributes those clusters with the similar or same SNR ratio profiles to the same subpopulation. Therefore, the variations in the SNR ratio profiles for each cluster can be monitored at each sequencing cycle, thereby achieving high accuracy and optimal performance for the base calling.

In other implementations, the SNR determination logic 504 can compute and store selected SNR ratio ranges for the clusters during at least one sequencing cycle. Instead of computing and storing each SNR ratio profile for each cluster at each sequencing cycle, the intensity profiles of the clusters within a selected SNR ratio range are analyzed. Clusters within the selected SNR ratio range provide substantially correct shapes of the four intensity distributions corresponding to the four bases A, G, C and T. Meanwhile, the selection of particular SNR ranges avoids the complexity in computation and data storage.

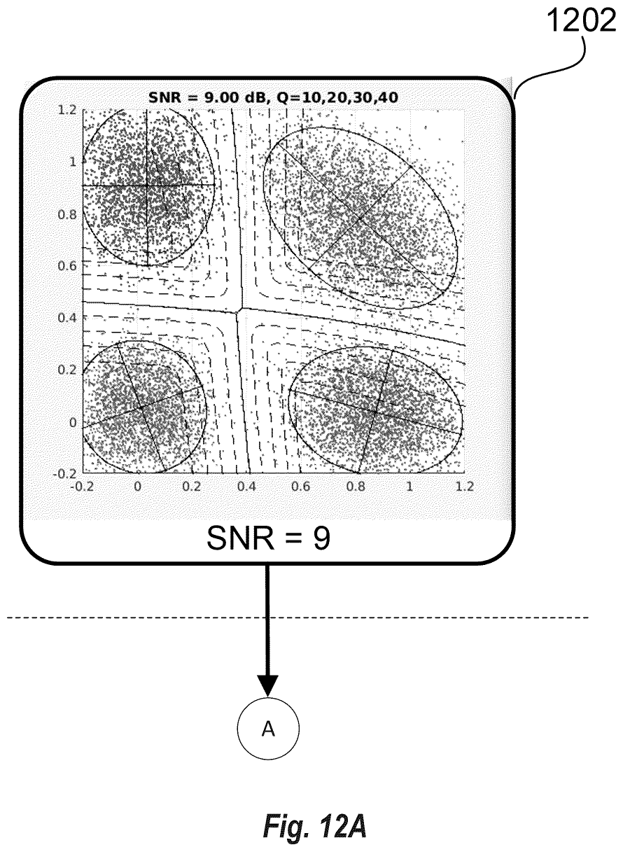

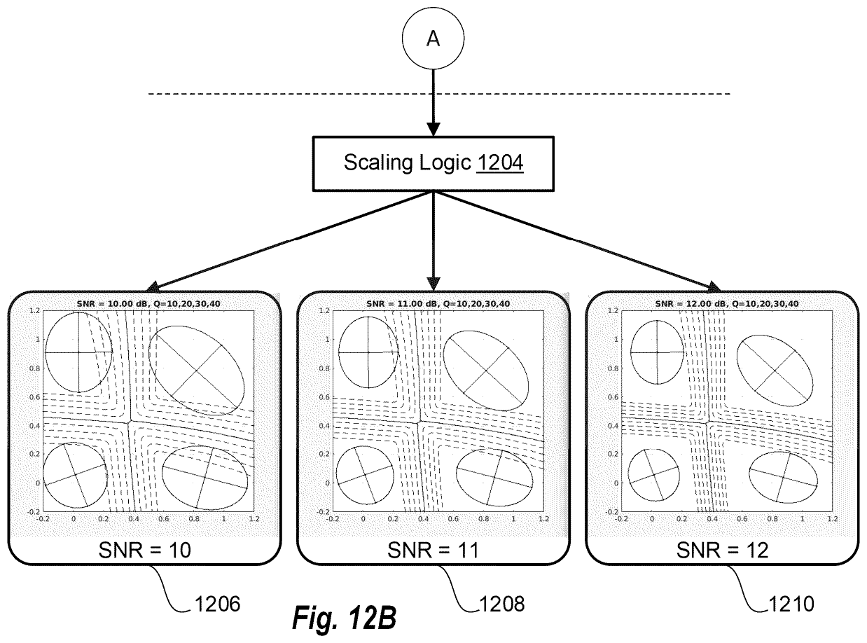

A scaling logic can be used to generate more intensity distributions representing the intensities of clusters with different SNR ratio profiles. FIGS. 12A-12B illustrate an example scaling logic that generates the intensity distributions representing the clusters with different SNR ratio profiles. The SNR ratio profile is selected, for example, to have a SNR ratio range with a SNR midpoint as 9. Those clusters having the selected SNR profiles are segmented, and the corresponding intensity profiles 1202 are generated by iteratively fitting a mixture of intensity distributions to the intensity values of the clusters. When the mixture of intensity distributions is a Gaussian mixture model, each of the four intensity distributions, corresponding to one of the four bases A, C, T, and G, has a centroid and covariances. The scaling logic 1204 scales the covariances of each of the four intensity distributions to generate scaled covariances that represent the intensity profiles of the clusters with different SNR profiles, for example, SNR=10 (1206), SNR=11 (1208) and SNR=12 (1210).

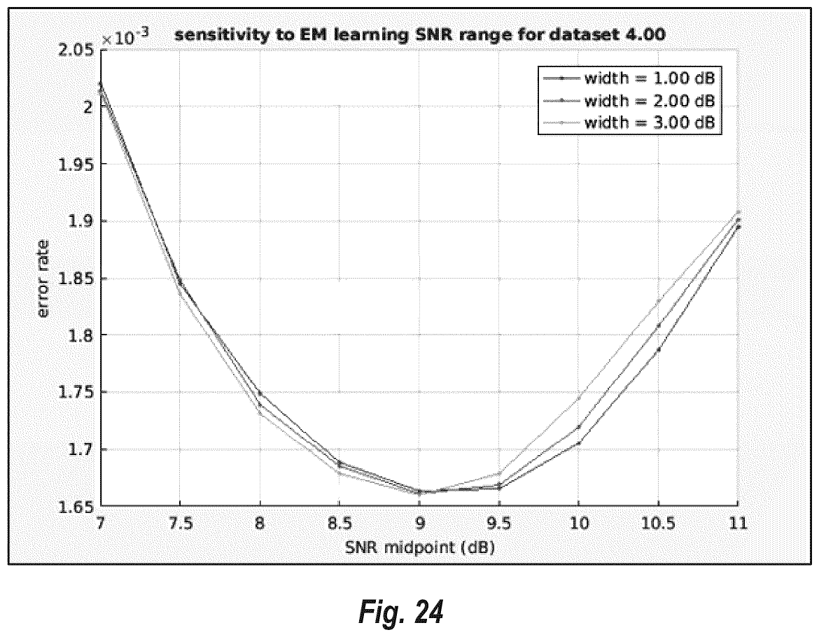

The SNR ratio ranges that are selected to attribute clusters for generating a corresponding mixture of intensity distributions can be optimized in order to minimize error rate of base calling. FIG. 24 illustrates the correlation between the selected SNR ratio ranges and the error rate of base calling. When the selected SNR midpoint varies between 7 dB and 11 dB, the error rates are represented by an approximately U-shaped curve. The error rates are also impacted by the selected SNR ratio ranges. For example, when a SNR midpoint is selected as 9 dB, the selected SNR ratio range can be 8.5-9.5 dB (with a width of 1.00 dB, shown in blue). Alternatively, the selected SNR ratio range can be 8-10 dB (with a width of 2.00 dB, shown in red), or 7.5 dB-10.5 dB (with a width of 3.00 dB, shown in yellow). For example, when the SNR midpoint is between 9 to 9.5 with a relatively small width (e.g., width=1.00 dB or 2.00 dB), the error rate of base calling is minimal.

When a target cluster is base called during a current sequencing cycle, based on its SNR profiles, a mixture of intensity distribution corresponding to the SNR profile is fitted to the intensity values of the target cluster. For example, when a target cluster to be base called has a particular SNR ratio range (e.g., SNR=9, 10, 11 and 12, respectively), a particular mixture of intensity distribution corresponding to the particular SNR ratio (e.g., 1206, 1208 and 1210, respectively) can be fitted to the intensity values of the target cluster to base call the cluster.

### Cluster Resegmentation

The segmentation logic 312/812 can resegment clusters into subpopulations at different sequencing cycles. The segmentation logic 312/812 can resegment a population of clusters into subpopulations at different intervals in the sequencing run. In some implementations, the different intervals correspond to successive sequencing cycles in the sequencing run. For example, the segmentation logic 312/812 can resegment the clusters into a plurality of subpopulations at each sequencing cycle. That is, clusters within each subpopulation are updated at each sequencing cycle. For a target cluster at a current sequencing cycle, it may be attributed to a particular subpopulation with a corresponding mixture of intensity distributions to base call the cluster. For the same target cluster during a succeeding sequencing cycle, it may be attributed to another subpopulation with a different mixture of intensity distributions.

The different intervals can correspond to non-successive sequencing cycles. The resegmentation can occur during alternative sequencing cycles, for example, cycles 1, 3, 5, . . . , and so on. The resegmentation can occur every N cycles, for example, at sequencing cycles 1, 11, 21, . . . , and so on. In some other implementation, the different intervals can correspond to blocks of sequencing cycles in the sequencing run. For example, the resegmentation occurs during sequencing cycles 1-5, 11-15, 21-25, . . . , and so on.

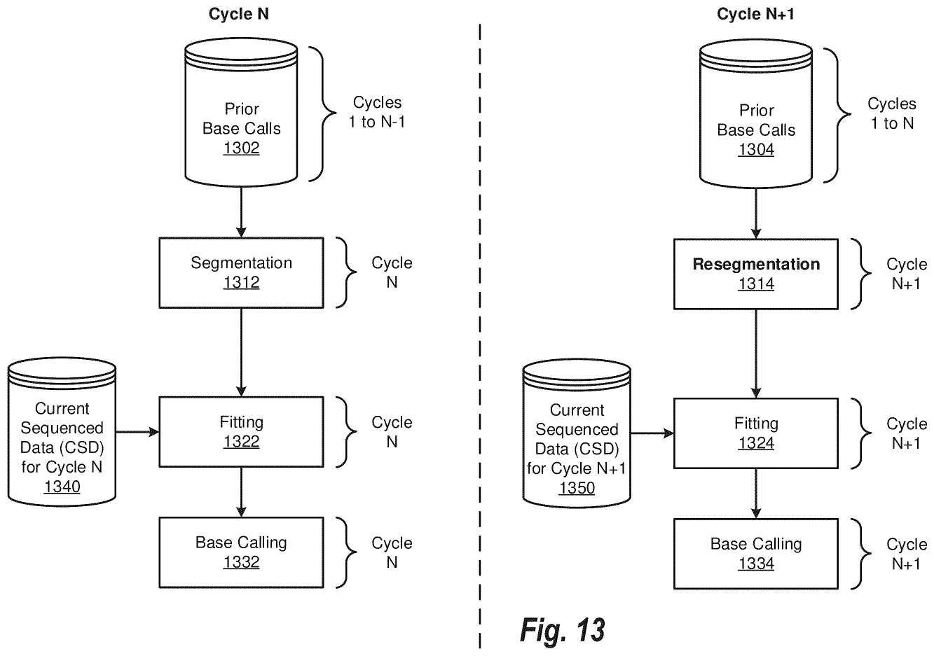

FIG. 13 illustrates an example workflow of resegmenting a population of clusters into subpopulations at different sequencing cycles. At current sequencing cycle N, the segmentation logic 312/812 performs segmentation 1312 to a population of clusters, based on the conditions of prior base calls 1302 identified at one or more prior sequencing cycles 1 to N−1. The conditions of prior base calls can include but not limited to prior base context, SNR ratio profiles, raw intensity profiles of the clusters, corrected intensity profiles of the clusters, types of signal variations detected in the intensity profiles of the clusters, values of inter-cluster intensity profile variation correction coefficients, etc.

Each of the subpopulations has a corresponding mixture of intensity distribution generated based on the intensity profiles of the clusters within the subpopulation during prior sequencing cycles 1 to N−1. For a target cluster within a given subpopulation at current sequencing cycle N, the fitting logic 352/852 fits a corresponding mixture of intensity distribution to the current sequenced data CSD 1340 to iteratively maximize the likelihood of the parameters of the mixture of the intensity distribution that best fit the current sequenced data (i.e., intensity profiles) of the target cluster (see, 1322). The base calling logic 372/872 base calls the target cluster based on the fitting (see, 1332). When the mixture of intensity distribution is a Gaussian mixture model, the centroid of the Gaussian distribution associated with the maximum likelihood value is determined as the base call for the target cluster.

At a next sequencing cycle N+1, the segmentation logic 312/812 performs resegmentation 1314 to the population of clusters, based on prior base calls 1304 identified at prior sequencing cycles 1 to N. The segmentation conditions may change from the prior sequencing cycle N to the next sequencing cycle N+1. Moreover, due to the newly added base calls identified at sequencing cycle N, the population of clusters to be resegmented is updated. As a result, the numbers of subpopulations and/or the clusters within each population can be different after the resegmentation. For the same target cluster, it may be attributed to a subpopulation during sequencing cycle N, yet to a different subpopulation during next sequencing cycle N+1. Accordingly, the fitting logic fits a mixture of intensity distributions corresponding to the subpopulation to which the target cluster belongs, to current sequenced data CSD 1350 (i.e., intensity profiles) at sequencing cycle N+1 for base calling (see, 1324 and 1334, respectively). Alternatively, the target cluster may be attributed to the same subpopulation, whereas this subpopulation includes different clusters at sequencing cycles N and N+1. The fitting logic 352/852 fits a mixture of intensity distributions corresponding to the updated subpopulation to which the target cluster belongs, to the intensity profiles of the target clusters during the sequencing cycle N+1 for base calling.

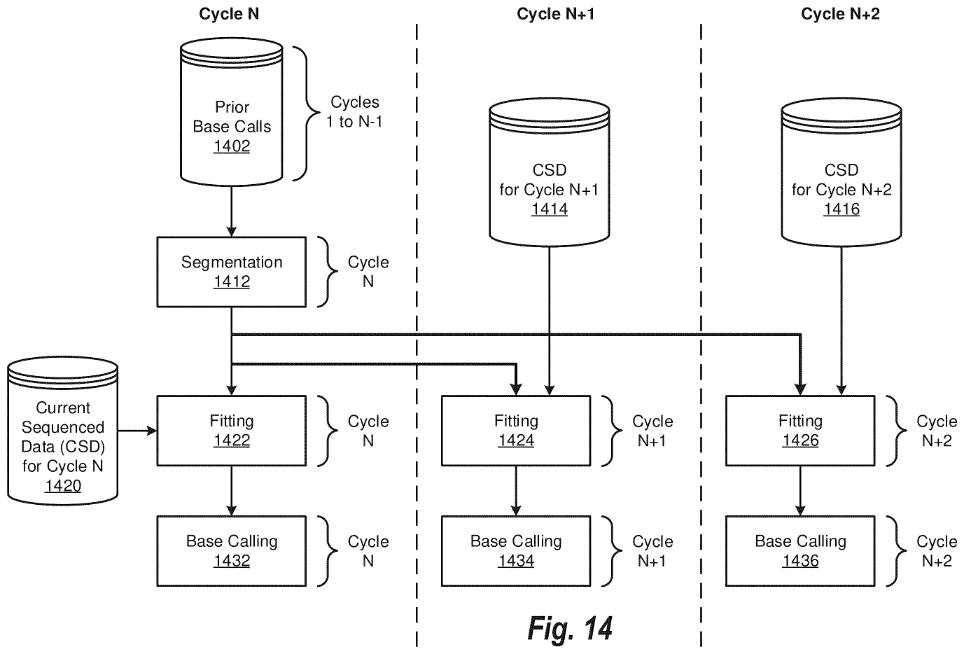

In some implementations, the resegmentation occurs at non-successive sequencing cycles. Each subpopulation of clusters is used for more than one sequencing cycle until the next resegmentation event occurs which updates the subpopulations of clusters. FIG. 14 illustrates another example workflow of resegmenting a population of clusters into subpopulations of clusters at different sequencing cycles. At current sequencing cycle N, the segmentation logic 312/812 performs segmentation 1412 to a population of clusters, based on the conditions of prior base calls 1402 identified at one or more prior sequencing cycles 1 to N−1. For a target cluster within a given subpopulation at current sequencing cycle N, the fitting logic 352/852 fits a corresponding mixture of intensity distribution to the current sequenced data CSD 1420 to iteratively maximize the likelihood of the parameters of the mixture of the intensity distribution that best fit the current sequenced data (i.e., intensity profiles) of the target cluster (see, 1422). The base calling logic 372/872 base calls the target cluster based on the fitting (see, 1432). These subpopulations of clusters generated from the segmentation process at current sequencing cycle, remain the same at succeeding sequencing cycles N+1 and/or N+2 before next round of segmentation. In other words, the clusters within a given subpopulation are the same at sequencing cycles N−1, N and N+1. At sequencing cycle N+1, the fitting logic 352/852 fits a corresponding mixture of intensity distributions to the current sequenced data CSD 1414 of the clusters within the given subpopulation for base calling (see, 1424, 1434). At sequencing cycle N+2, the fitting logic 352/852 fits a corresponding mixture of intensity distributions to the current sequenced data CSD 1416 of the clusters within the given subpopulation for base calling (see, 1426, 1436).

In other implementations, the resegmentation process is optional. That is, the segmentation may occur only once during a sequencing run. For example, when a population of clusters is segmented based on different types of input library or insert lengths, the segmentation can occur at a first sequencing cycle of the sequencing run.

### Performance Results of Conditional Base Calling

### Base Calling Conditioned on Prior Base Context

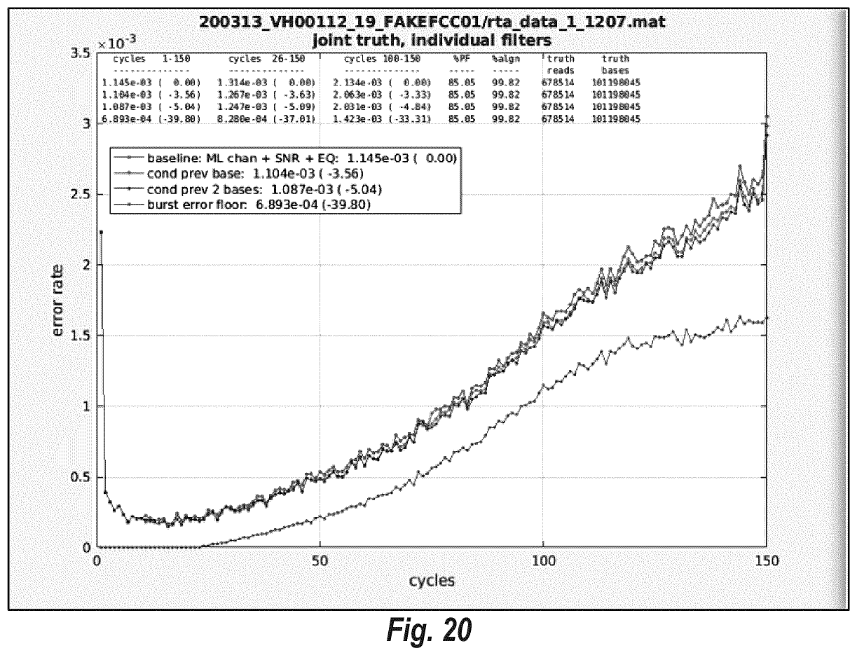

FIGS. 20 and 21 are performance results of base calling by segmenting clusters into subpopulations based on prior base context. Real-time analysis (RTA) without cluster segmentations is used as a benchmark model. FIG. 20 illustrates the performance results of base calling at 150 sequencing cycles at a sequencing run, by segmenting a population of clusters based on a single prior base call and two prior base calls. The burst error floor is illustrated in grey (“burst error floor”). The error rate of the RTA benchmark model is illustrated in blue (“baseline: ML chan+SNR+EQ”). The error rate of base calling conditioned on a single prior base and on two prior bases are illustrates in red (“cond prev base”) and green (“cond prev 2 bases”), respectively. Compared to RTA benchmark model, the error rate of base calling conditioned on a single prior base is reduced by 3.56%, and the error rate of base calling conditioned on two prior bases is reduced by 5.04%.

We found when soft-clipping errors are removed, the error rate of base calling conditioned on prior base context is further reduced. Soft-clipping of reads indicates that portions of the read that do not match well to the reference genome on either side of the read are ignored for the alignment as such. Soft-clipping errors are generated when the reads are improperly soft-clipped. FIG. 21 illustrates when soft-clipping errors are removed, the error rate of base calling conditioned on prior base context is significantly reduced. For example, compared to RTA benchmark model illustrated in blue (“baseline: ML chan+SNR+EQ”), the error rate of base calling conditioned on a single prior base is reduced by 9% (“cond prev base”), and the error rate of base calling conditioned on two prior bases is reduced by 12.5% (“cond prev 2 bases”).

### Base Calling Conditioned on SNR Ratio Profiles

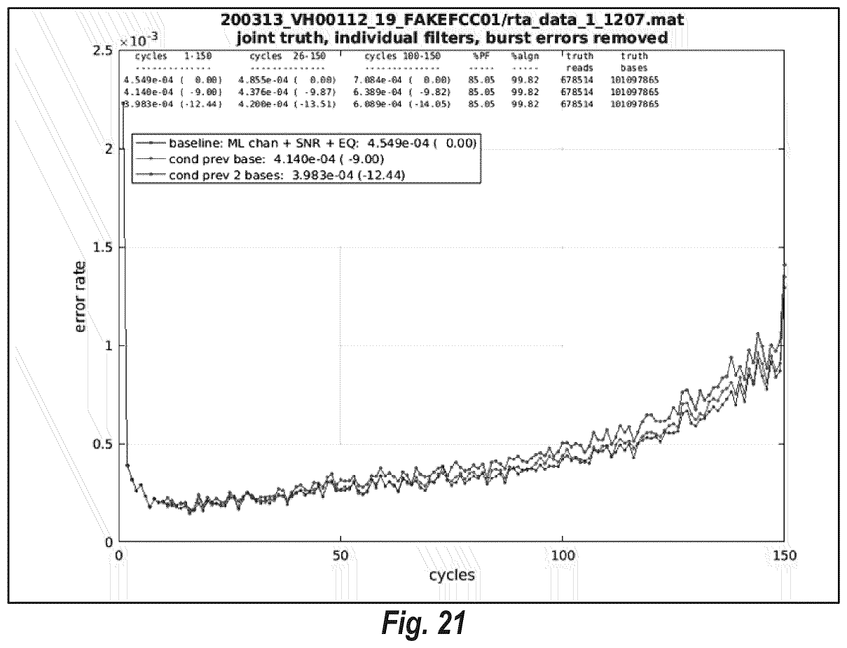

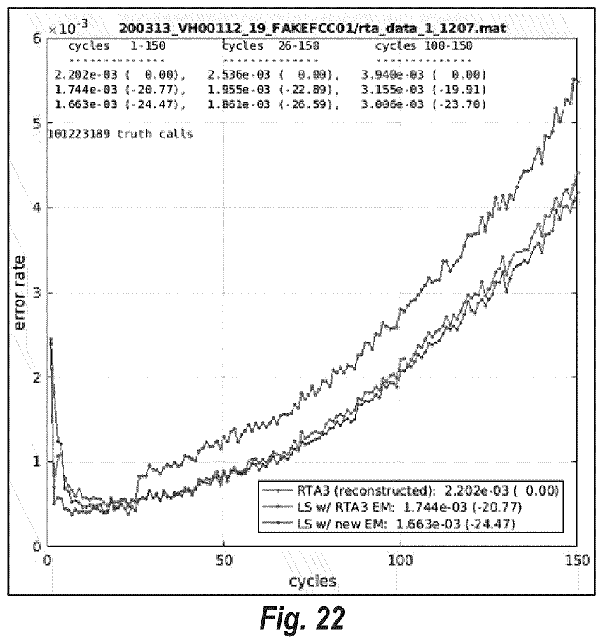

FIG. 22 illustrates performance results of base calling conditioned on SNR ratio profiles of clusters. The reconstructed RTA3 model (“RTA3 reconstructed”) is used as benchmark and its error rate of base calling is illustrated in blue. The error rate of the RTA3 model using least square channel estimation but without conditioning on SNR ratios (“LS w/RTA3 EM”) is illustrated in red, whereas the RTA3 model using least square channel estimation and conditioning on SNR ratios (“LS w/ new EM”) is illustrated in green. Compared to the RTA3 model using least square channel estimation but without conditioning on SNR ratio profiles, the conditioning reduces the error rate by approximately 5%.

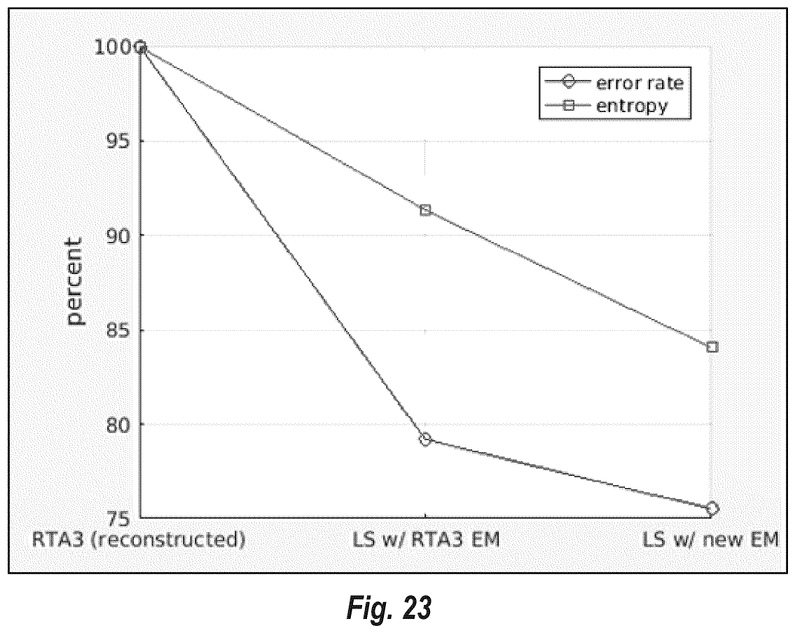

FIG. 23 illustrates performance results of base calling in error rate and entropy conditioned on SNR ratio profiles of clusters. Compared to the reconstructed RTA3 model, the base calling approach conditioned on SNR ratio profiles (“LS w/ new EM”) reduced the error rate by 25%. Compared to the RTA3 model using least square channel estimation (LS w/RTA3 EM), the conditioning on SNR ratios further reduces error rate by approximately 5%. Furthermore, the entropy of the base calling approach conditioned on SNR ratio profiles of clusters is reduced by over 15% compared to the reconstructed RTA3 model, and reduced by approximately 7% compared to the RTA3 model using least square channel estimation.

### High-Dimensional Mixture of Distributions for Base Calling

Next, we turn to an alternative implementation of taking into consideration prior base context during base calling. In the aforementioned implementations, a population of clusters is segmented into various subpopulations of clusters, where each subpopulation has a corresponding mixture of intensity distributions used to base call the clusters within the subpopulation. In the case where prior base call context is considered, for example, prior base calls are already identified at prior sequencing cycles, the segmentation logic 312/812 can segment the clusters by the identified prior base calls. Here, we introduce a high-dimensional mixture of intensity distributions to perform base calls simultaneously for at least two sequencing cycles. In some implementations, the current intensity profiles of a population of clusters at current sequencing cycle and the prior intensity profiles at a number k of prior sequencing cycles are processed by applying a high-dimensional mixture of distributions that includes 4 k+1 intensity distributions. The 4 k+1 intensity distributions correspond to 4 k+1 permutations of (i) k base calls at k prior sequencing cycles based on the prior intensity profiles and (ii) one base call at current sequencing cycle based on the current intensity profiles.

For a target cluster to be base called, its intensity profiles at each of the k prior sequencing cycles and current sequencing cycle are extracted from the sequencing images acquired from each color/intensity channel. Since one base is called for the target cluster at each sequencing cycle, there are k+1 bases that are to be identified. The fitting logic 312/812 fits the high-dimensional mixture of distributions to the intensity profiles of the target cluster, to determine the likelihoods of the intensity profiles of the target cluster belongs to each of the 4 k+1 distributions. Because each of the 4 k+1distributions represents a particular combination of k+1 bases, the distribution that best fits the intensity profiles of the target cluster determines simultaneously the k+1 bases for the target cluster.

Compared to the approaches of cluster segmentation and separate base calling on a subpopulation-by-subpopulation basis, the high-dimensional base calling approach can simultaneously base call clusters at current sequencing cycle as well as prior sequencing cycles. The high-dimensional base calling approach may not need segmenting the cluster population, generating mixtures of intensity distributions corresponding to each subpopulation, or separately fitting the corresponding mixture of intensity distributions for base calling.

We now turn to explaining the dimensions of the mixtures of intensity distributions. Consider a scenario where a population of clusters is to be base called, taking into consideration a single prior base during a prior sequencing cycle. That is, the clusters are to be base called at current sequencing cycle as well as prior sequencing cycle. The current intensity profiles of the clusters at current sequencing cycle via two intensity channels and prior intensity profiles at prior sequencing cycle via the two intensity channels are used to generate a four-dimensional mixture of intensity distributions. Similarly, if the clusters are to be base called at current sequencing cycle as well as two prior sequencing cycles, the current intensity profiles of each cluster at current sequencing cycle via two intensity channels and two intensity profiles at two prior sequencing cycle via the two intensity channels are used to generate a six-dimensional mixture of intensity distributions.

In one implementation, the high-dimensional mixture of intensity distributions can be a high-dimensional Gaussian distribution. For a D-dimensional vector x, the multivariant Gaussian distribution takes the form of

$$𝒩 ( x \vert μ , Σ ) = \frac{1}{{( 2 π )}^{D / 2}} \frac{1}{{\vert Σ \vert}^{1 / 2}} exp \{ - \frac{1}{2} {( x - μ )}^{T} {Σ}^{- 1} ( x - μ ) \} , \qquad ( 1 )$$

where μ is a D-dimensional mean vector, Σ is a D×D covariance matrix, and |Σ| denotes the determinant of Σ.

Other algorithms for grouping high-dimensional datapoints can be used to generate intensity distributions for the four nucleotide bases A, G, C and T, including k-means clustering algorithm, mean-shift clustering algorithm, density-based spatial clustering of applications with noise (DBSCAN), agglomerative hierarchical clustering algorithm.

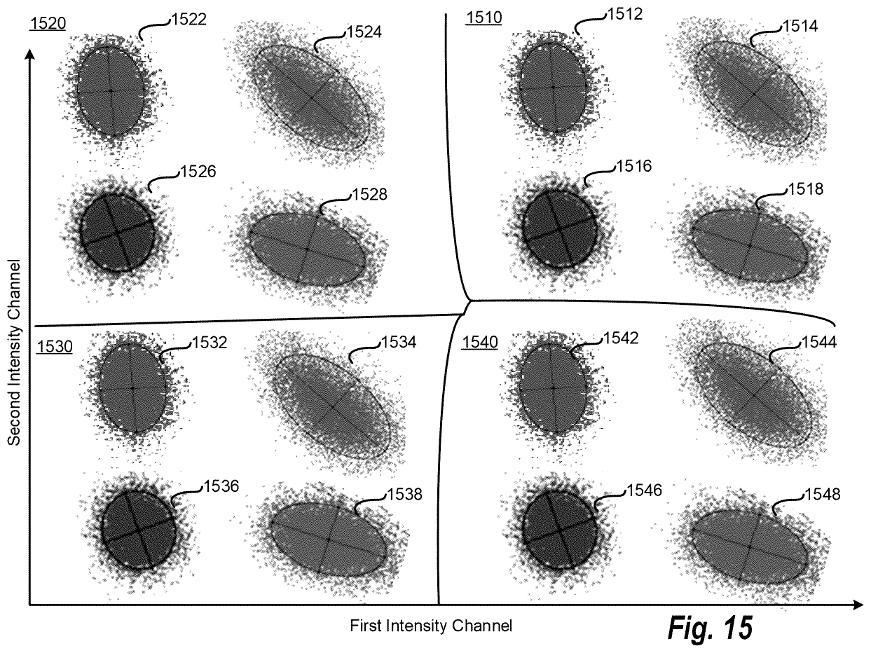

FIG. 15 illustrates an example high-dimensional mixture of intensity distributions. A population of clusters is to be base called at current sequencing cycle N and a prior sequencing cycle N−1. In a four-dimensional space, the mixture of intensity distributions include sixteen distributions, corresponding to sixteen combinations of base calls at current sequencing cycle N and prior sequencing cycle N−1, namely, AA, AG, AC, AT, CA, CG, CC, CT, GA, GG, GC, GT, TA, TG, TC and TT. As illustrated, the sixteen combinations can be categorized into four categories, each category corresponding to one of the four bases A, G, C and T at current sequencing cycle. Category A 1510 corresponds to all clusters that are base called as A at current sequencing cycle. Category C 1520 corresponds to all clusters that are based called as C at current sequencing cycle. Category G 1530 corresponds to all clusters that are base called as G at current sequencing cycle. Category T 1540 corresponds to all clusters that are based called as T at current sequencing cycle.

Each category includes four distributions, each corresponding to the current base call and a particular prior base call identified at prior sequencing cycle. Category A 1510 includes distribution 1512 corresponding to two bases CA, where C is called at prior sequencing cycle and A is called at current sequencing cycle. Similarly, distribution 1514 corresponds to two bases AA, where base A is called at both prior and current sequencing cycles. Distribution 1516 corresponds to two bases GA, where G is called at prior sequencing cycle and A is called at current sequencing cycle. Distribution 1518 corresponds to two bases TA, where T is called at prior sequencing cycle and A is called at current sequencing cycle. Category C 1520 includes four distributions 1522, 1524, 1526 and 1528. Distribution 1522 corresponds to two bases CC, where base C is called at prior and current sequencing cycles. Distribution 1524 corresponds to two bases AC, where base A is called at prior sequencing cycle and base C called at current sequencing cycle. Distribution 1526 corresponds to two bases GC, where G is called at prior sequencing cycle and C is called at current sequencing cycle. Distribution 1528 corresponds to two bases TC, where T is called at prior sequencing cycle and C is called at current sequencing cycle. Category G 1530 includes four distributions 1532, 1534, 1536 and 1538. Distribution 1532 corresponds to two bases CG, where base C is called at prior sequencing cycle and base G called at current sequencing cycles. Distribution 1534 corresponds to two bases AG, where base A is called at prior sequencing cycle and base G called at current sequencing cycle. Distribution 1536 corresponds to two bases GG, where G is called at both prior and current sequencing cycles. Distribution 1538 corresponds to two bases TG, where T is called at prior sequencing cycle and G is called at current sequencing cycle. Category T 1540 includes four distributions 1542, 1544, 1546 and 1548. Distribution 1542 corresponds to two bases CT, where base C is called at prior sequencing cycle and base T called at current sequencing cycles. Distribution 1544 corresponds to two bases AT, where base A is called at prior sequencing cycle and base T called at current sequencing cycle. Distribution 1546 corresponds to two bases GT, where base G is called at prior sequencing cycle and base T called at current sequencing cycles. Distribution 1548 corresponds to two bases TT, where base T is called at both prior and current sequencing cycles.

For a target cluster to be base called at prior sequencing cycle N−1 and current sequencing cycle N, the fitting logic fits the high-dimensional mixture of intensity distributions to the intensity profiles of the target clusters at the cycles N−1 and N. For example, distribution 1542 is determined to be the best fit for intensity profiles of the target cluster. Accordingly, bases C and T, corresponding to the distribution 1542, are called at prior sequencing cycle and current sequencing cycle, respectively.

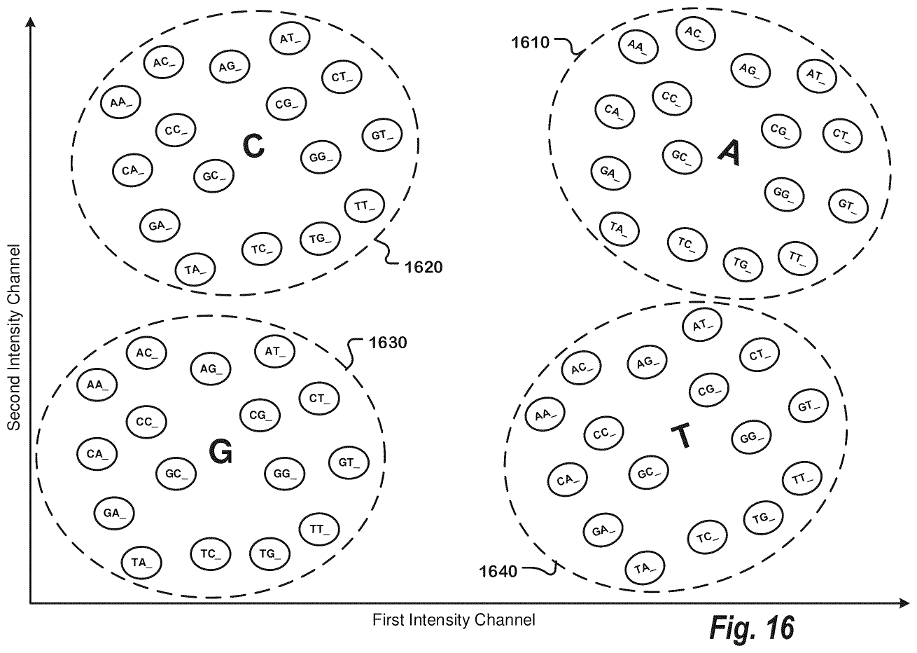

FIG. 16 is another example high-dimensional mixture of intensity distributions. A population of clusters is to be base called at current sequencing cycle N and two prior sequencing cycles N−1 and N−2. In a six-dimensional space, the mixture of intensity distributions includes sixty-four distributions, corresponding to sixty-four combinations of base calls at sequencing cycles N−2, N−1 and N. The sixty-four distributions include AAA, ACA, AGA, ATA, CAA, CCA, CGA, CTA, GAA, GCA, GGA, GTA, TAA, TCA, TGA, TTA, AAC, ACC, AGC, ATC, CAC, CCC, CGC, CTC, GAC, GCC, GGC, GTC, TAC, TCC, TGC, TTC, AAG, ACG, AGG, ATG, CAG, CCG, CGG, CTG, GAG, GCG, GGG, GTG, TAG, TCG, TGG, TTG, AAT, ACT, AGT, ATT, CAT, CCT, CGT, CTT, GAT, GCT, GGT, GTT, TAT, TCT, TGT, TTT.

As illustrated in FIG. 16, the sixty-four distributions can be categorized into four categories, each category corresponding to one of the four bases A, G, C and T at current sequencing cycle. Category A 1610 corresponds to those clusters that are base called as A at current sequencing cycle. Category C 1620 corresponds to those clusters that are based called as C at current sequencing cycle. Category G 1630 corresponds to those clusters that are base called as G at current sequencing cycle. Category T 1640 corresponds to those clusters that are based called as T at current sequencing cycle.

Each category includes four distributions, each corresponding to the current base call and two particular prior base calls identified at two prior sequencing cycles. Category A 1610, representing clusters that are base called as A at current sequencing cycle, includes sixteen distributions of combinations of two prior base calls at two prior sequencing cycles, namely, AA_, AG_, AC_, AT_, CA_, CG_, CC_, CT_, GA_, GG_, GC_, GT_, TA_, TG_, TC_ and TT_. Similarly, category C 1620, category G 1630 and category T 1640 each includes sixteen distributions of combinations of two prior base calls at two prior sequencing cycles.

For a target cluster to be base called at two prior sequencing cycles N−2, N−1 and current sequencing cycle N, the fitting logic 352/852 fits the six-dimensional mixture of intensity distributions to the intensity profiles of the target clusters at the cycles N−2, N−1 and N. For example, distribution CA_ in the category A 1610 is determined to be the best fit for the intensity profiles of the target cluster. Accordingly, bases C, A and A are called at sequencing cycle N−2, N−1 and N, respectively.

For the sake of simplicity, FIGS. 15 and 16 are illustrated on a two-dimensional plot. A person skilled in the art will appreciate the two-dimensional plot is used only for illustrative purposes and is intended to cover the four-dimensional mixtures of intensity distributions for FIG. 15 and six-dimensional mixtures of intensity distributions for FIG. 16, respectively.

### Correction of Parameters of Mixture of Intensity Distributions

We describe herein an alternative approach of base calling target clusters taking into consideration prior base context by correcting the parameters of the mixture of intensity distributions. As prior base context influences the intensity profiles for the clusters at current sequencing cycle, the clusters based on different prior base context can be segmented and the parameters (e.g., centroids) of each corresponding mixture of intensity distributions can be calculated. These parameters can be used to correct for the base calling at current sequencing cycle.

In some implementations, the segmentation logic 312/812 segments a population of clusters into 4k subpopulations of clusters based on k prior bases called at k prior sequencing cycles of the sequencing run (k=1, 2, 3, 4 . . . ). For example, when the segmentation is based on a single prior base called at a prior sequencing cycle of the sequencing run, the segmentation logic segments the population of clusters into four subpopulations of clusters. Each subpopulation includes those clusters that had an A, G, C or T base call at prior sequencing cycle. Alternatively, when the segmentation is based on two prior bases called at prior sequencing cycles, the segmentation logic segments the population of clusters into sixteen subpopulations of clusters. Alternatively, when the segmentation is based on three prior bases called at prior sequencing cycles, the segmentation logic segments the population of clusters into sixty-four subpopulations of clusters.

The intensity profiles of the clusters within each subpopulation can be processed and fitted to a mixture of intensity distributions. For example, the segmentation logic 312/812 segments a population of clusters into sixty-four subpopulations based on three prior bases called at prior sequencing cycles. Each cluster within a given subpopulation can be called as one of the four bases A, G, C or T at current sequencing cycle and thus, a mixture of four intensity distributions can be fitted to the intensity profiles of the clusters within the given subpopulation. For those clusters that are called as the same base at current sequencing cycle, their intensity profiles at each intensity channel can be averaged, thereby generating an averaged intensity profile corresponding to the base. When the mixture of four intensity distributions is a Gaussian mixture model, the averaged intensity profile corresponds to the mean values that defines the centroids of the Gaussian distribution. Since each subpopulation has a corresponding Gaussian mixture model with four centroids, sixty-four subpopulations have two hundred and fifty-six centroids.

For those clusters that are called as the same base at current sequencing cycle but with different prior base context, their averaged intensity profiles (i.e., centroids) can be ranked. For example, for those clusters that are called as base A at current sequencing cycle but with sixty-four different trimer (three consecutive bases) context, sixty-four intensity profiles (i.e., centroids) at a given intensity channel can be ranked. Each of the sixty-four intensity profiles can be compared to a median or mean intensity profile and generates a corresponding offset value at the given intensity channel. That is, for those clusters that are called as the same base at current sequencing cycle but with different two prior base context, there are a total of sixteen channel-specific offset values. For those clusters that are called as the same base at current sequencing cycle but with different trimer context, there are a total of sixty-four channel-specific offset values. These offsets are summary statistics determined from subpopulation-wise sequenced data (i.e., intensity profiles).

For a target cluster to be base called at current sequencing cycle, its prior base context at prior sequencing cycles are known. The intensity profiles of the target cluster at current sequencing cycle can be corrected using offset values corresponding to the prior base context that the target cluster has. The corrected intensity profiles of the target clusters can be used to base call the target cluster.

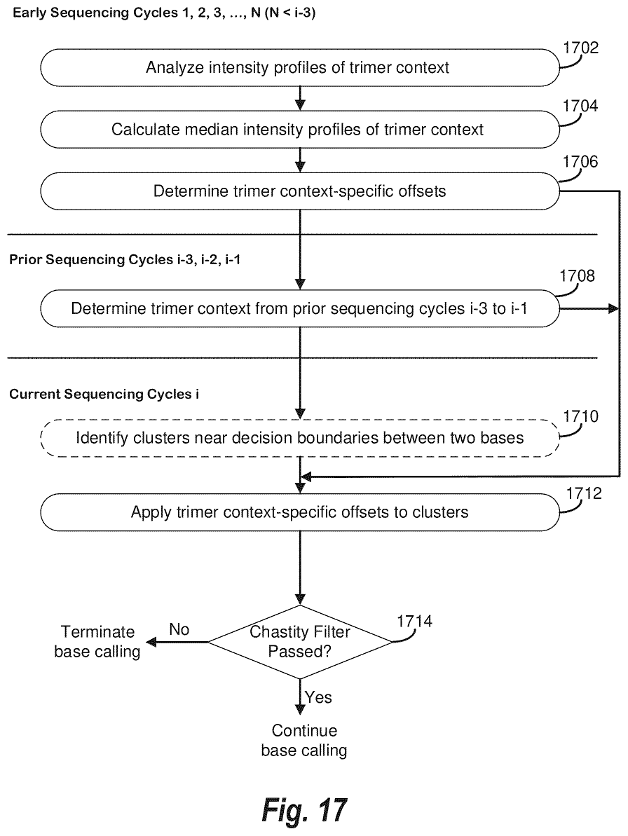

FIG. 17 illustrates an example workflow of correcting the intensity profiles of clusters at current sequencing cycle based on prior base context identified at prior sequencing cycles. At early sequencing cycles 1, 2, 3, . . . , N (e.g., N<i−3), a population of clusters are segmented into a plurality of subpopulations based on trimer context at prior sequencing cycles. For example, for all the clusters that are based called as “A” at a given sequencing cycle, the segmentation logic 312/812 segments those clusters into sixty-four subpopulations based on their prior trimer context identified at three sequencing cycles proceeding the given sequencing cycle.

At step 1702, for the clusters within each of the sixty-four subpopulations, their intensity profiles at each intensity channel are analyzed and ranked. For example, the intensity profiles of the clusters within each of the sixty-four subpopulations can be averaged to generate an averaged channel-specific intensity profile. Hence, there are a total of sixty-four channel-specific averaged intensity profiles.

At step 1704, by ranking the sixty-four averaged channel-specific intensity profiles, a median intensity profile is identified. Alternatively, a mean intensity profile by averaging the sixty-four averaged channel-specific intensity profiles can be calculated.

At step 1706, for each of the sixty-four subpopulations, a corresponding channel-specific offset value is calculated by comparing the channel-specific averaged intensity profiles corresponding to the subpopulation with the median or mean intensity profile. Hence, there are a total of sixty-four channel-specific offset values.

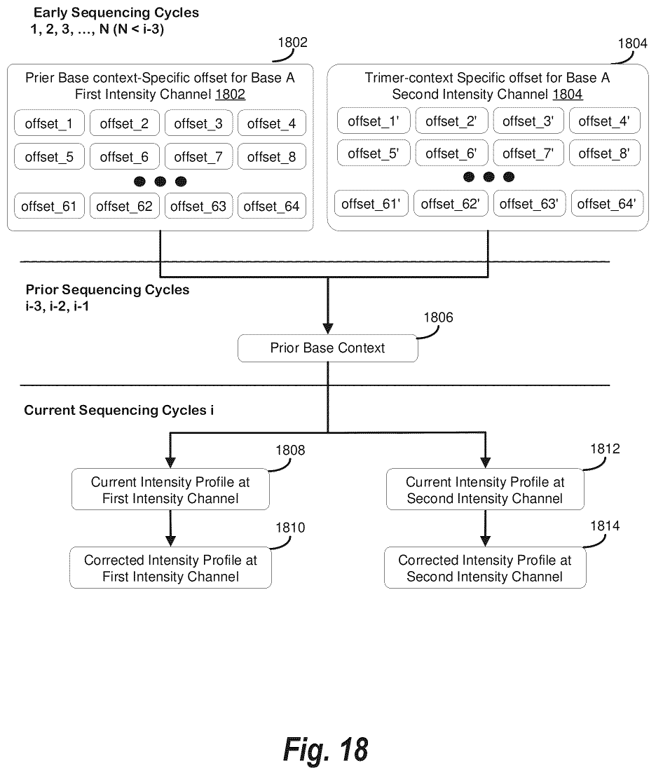

FIG. 18 is another example workflow of correcting the intensity profiles of clusters at current sequencing cycle based on prior base context identified at prior sequencing cycles. At early sequencing cycles 1, 2, 3, . . . , N (N<i−3), for all the clusters that are based called as “A” at a given sequencing cycle, the segmentation logic 312/812 segments those clusters into sixty-four subpopulations based on their prior trimer context identified at three sequencing cycles proceeding the given sequencing cycle. Accordingly, there are sixty-four trimer context-specific offset values (1802) for the first intensity channel, namely, offset_1, offset_2, . . . , offset_64. Each offset value corresponds to a particular subpopulation of clusters with a given trimer context AAA, ACA, AGA, ATA, CAA, CCA, CGA, CTA, GAA, GCA, GGA, GTA, TAA, TCA, TGA, TTA, AAC, ACC, AGC, ATC, CAC, CCC, CGC, CTC, GAC, GCC, GGC, GTC, TAC, TCC, TGC, TTC, AAG, ACG, AGG, ATG, CAG, CCG, CGG, CTG, GAG, GCG, GGG, GTG, TAG, TCG, TGG, TTG, AAT, ACT, AGT, ATT, CAT, CCT, CGT, CTT, GAT, GCT, GGT, GTT, TAT, TCT, TGT or TTT, respectively. Similarly, there are sixty-four trimer context-specific offset values (1804) for the second intensity channel, namely, offset_1′, offset_2′, . . . , offset_64′. Each offset value corresponds to a particular subpopulation of clusters with a given trimer.

At step 1708, target clusters are base called at prior sequencing cycles i-3, i-2 and i-1, which in turn, determines the trimer context. As illustrated in FIG. 18, the given trimer context 1806 is used to identify the corresponding channel-specific offset values. Consider an example the given trimer context 1806 of a target cluster identified at prior sequencing cycles i-3 to i-1 is ATA. Accordingly, offset_4 at the first intensity channel and offset_4′ at the second intensity channel are identified as the corresponding channel-specific offset values for the target cluster.

At step 1712, the corresponding channel-specific offset values are applied to the intensity profiles of the clusters at current sequencing cycle i. As illustrated in FIG. 18, the corresponding channel-specific offset values are applied to the current intensity profile 1808 at the first intensity channel and the current intensity profile 1812 at the second intensity channel, respectively, to generate corrected intensity profiles 1810 and 1814.

At step 1714, a chastity filter is applied to the corrected intensity profiles. Chastity is defined as the ratio of the brightest base intensity divided by the sum of the brightest and second brightest base intensities. Clusters are deemed to pass the chastity filter if no more than one base call has a chastity value below 0.6 in the first twenty-five cycles. This filtration process removes the least reliable clusters from the image analysis results. The corrected intensity profiles that pass the chastity filter is used for base calling. Otherwise, the base calling process is terminated.

Optionally at step 1710, the clusters with intensity profiles at current sequencing cycle i near decision boundaries between two bases are identified. These clusters may contribute to a high error rate of base calling. Correcting the intensity profiles of these clusters can effectively move the intensities away from the decision boundaries such that they can be correctly base called.

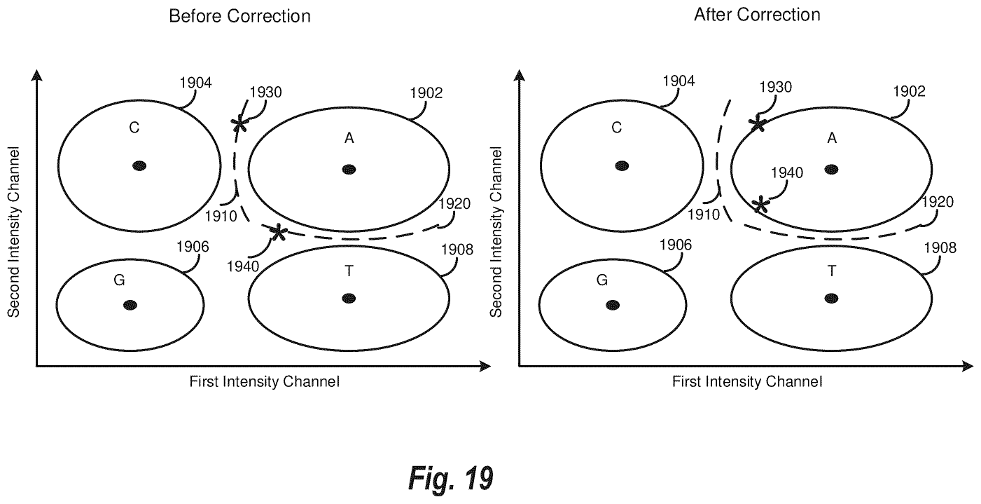

FIG. 19 illustrates an example comparison of the intensity profiles of clusters before and after correction. Before correction, the intensity profiles of target cluster 1930 fall onto the decision boundary line 1910, which is located between the intensity distribution 1904 corresponding to base C and the intensity distribution 1902 corresponding to base A. Similarly, the intensity profiles of target cluster 1940 fall on the decision boundary line 1920 between the intensity distribution 1902 corresponding to base A and the intensity distribution 1908 corresponding to base T. As shown in FIG. 19, the decision boundary lines 1910 and 1920 do not concern the intensity distribution 1906 corresponding to base G. After applying channel-specific offset values based on the trimer context of the clusters, the corrected intensity profiles of target cluster 1930 are shifted at a substantially horizontal direction, and the intensity profiles of target cluster 1940 are shifted a substantially vertical direction. Accordingly, the intensity profiles of target clusters 1930 and 1940 are away from the decision boundary lines 1910 and 1920 and correctly called for base A at current sequencing cycle.

### Performance Results

We now turn to the performance results of correcting the intensity profiles of target clusters based on prior base context identified at prior sequencing cycles. Consider as an example those clusters that are called as base A at a given sequencing cycle. Their trimer context at three prior sequencing cycles proceeding the given sequencing cycle is identified and based on which, those clusters are segmented into sixty-four subpopulations, each corresponding to a particular trimer context.

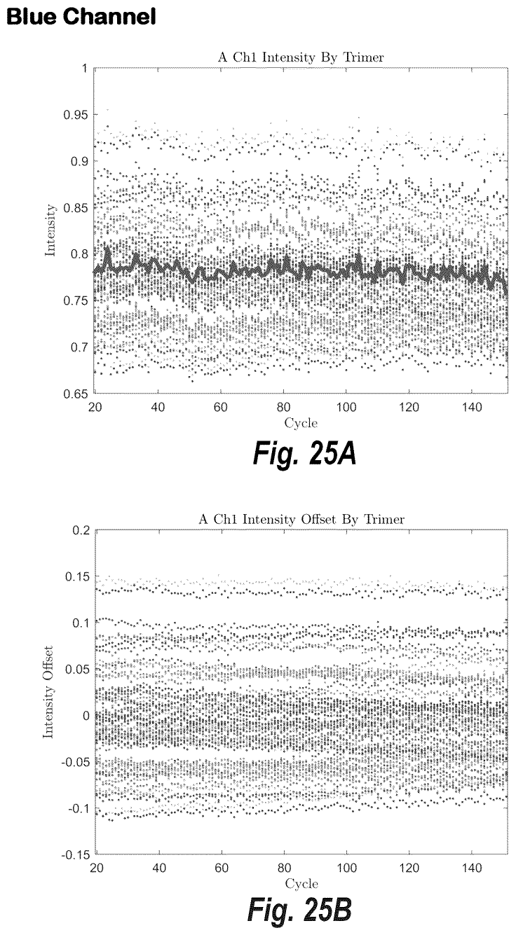

FIG. 25A illustrates the intensity profiles of clusters within each of the sixty-four subpopulations captured at the first intensity channel (e.g., blue channel) over a plurality of sequencing cycles. The bold red line represents a median intensity profile by ranking the sixty-four intensity profiles. FIG. 25B illustrates the offset values corresponding to the sixty-four subpopulations at the first intensity channel by applying the median intensity profile. The prior trimer context causes significant shift in the intensity values, varying from −0.1 to 0.15 intensity unit at the first intensity channel.

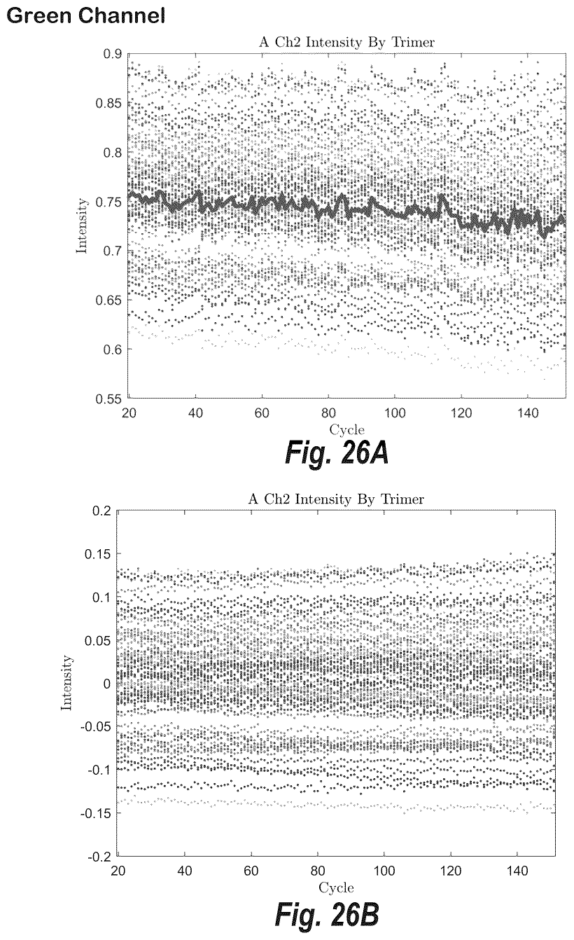

FIG. 26A illustrates the intensity profiles of clusters within each of the sixty-four subpopulations captured at the second intensity channel (e.g., green channel) over a plurality of sequencing cycles. Similar to FIG. 25A, the bold red line represents a median intensity profile by ranking the sixty-four intensity profiles. FIG. 26B illustrates the offset values corresponding to the sixty-four cluster subpopulations at the second intensity channel by applying the median intensity profile. The prior trimer context causes significant shift in the intensity values, varying from −0.15 to 0.15 intensity unit at the second intensity channel.

When a prior trimer context causes a negative intensity offset, it is more likely to cause incorrect base calls at current sequencing cycle. Consider as examples the two target clusters 1930 and 1940 in FIG. 19. Their prior trimer context causes negative intensity offset which in turn, causes the intensity profiles to move away from the correct base (i.e., base A at current sequencing cycle) towards a different but incorrect base. In particular, cluster 1930 is moved toward base C because its prior trimer context causes a negative intensity offset at the first intensity channel. Cluster 1940 is moved toward base T because its prior trimer context causes a negative intensity offset at the second intensity channel.

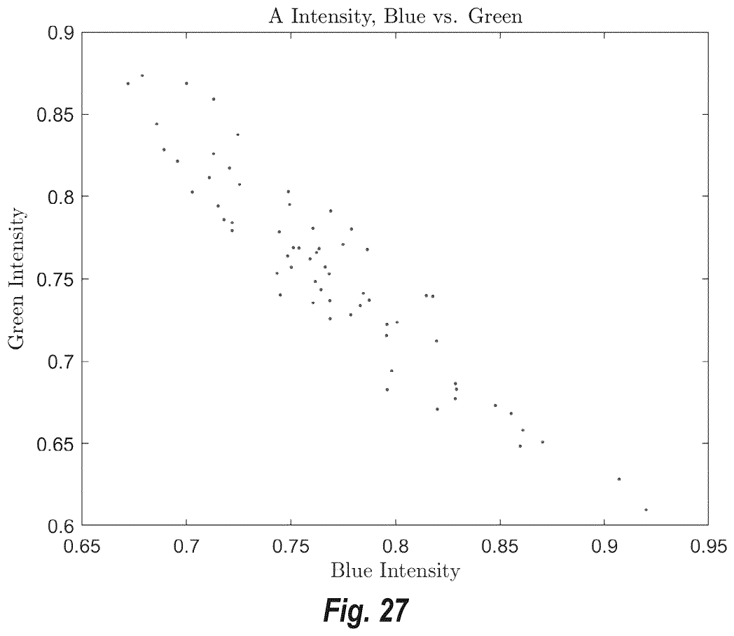

FIG. 27 illustrates the intensity correlation between two intensity channels for each of the sixty-four subpopulations. Each data point represents the intensity profiles of a particular trimer at the first and second intensity channels (e.g., blue and green channels, respectively). The intensities captured at two intensity channels are anti-correlated. In other words, some trimer context may cause a substantial offset at the first intensity channel while other trimer context causes at the second intensity channel. It is also consistent with the examples in FIG. 19 where the prior trimer context corresponding to cluster 1930 caused the intensity profiles to shift from base A toward base C along the first intensity channel while the intensity profiles of cluster 1940 is shifted from base A toward base T along the second intensity channel.

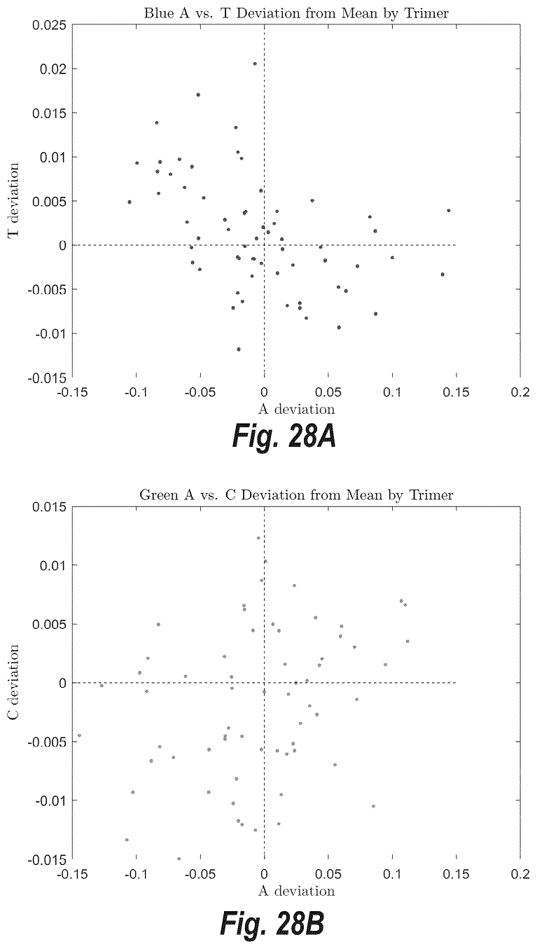

FIGS. 28A and 28B depict the deviations in intensity profiles of “ON” base and “OFF” bases. A “ON” base refers to a base (e.g., base A) with optical labels that generate intensity values at both intensity channels. “OFF” bases refer to bases with optical labels that generate intensity values at only one intensity channel (e.g., bases C and T), or bases that lack labels and thus, have no or minimal signals detected at either intensity channel (e.g., base G). FIG. 28A illustrates the intensity deviation caused by prior trimer context of the clusters that are called as base A and the clusters that are called as base T. For those clusters that are called as base A at a given sequencing cycle, they are segmented into sixty-four subpopulations, each subpopulation representing a particular trimer context identified at prior sequencing cycles proceeding the given sequencing cycle. For each subpopulation, the intensity offset/deviation (“A deviation” at x-axis) at the first intensity channel is calculated by comparing the intensity profiles corresponding to the subpopulation with a mean intensity value. Similarly, those clusters that are called as base T at a given sequencing cycle are segmented into sixty-four subpopulations, each subpopulation representing a particular trimer context identified at prior sequencing cycles proceeding the given sequencing cycle. For each subpopulation, the intensity offset/deviation (“T deviation” at y-axis) at the first intensity channel is calculated by comparing the intensity profiles corresponding to the subpopulation with a mean intensity value. The deviation caused by prior trimer context when the current base is A is in the range of −0.1 to 0.15 intensity unit, almost ten times more than the deviation caused by prior trimer context when the current base is T. In other words, prior trimer context that leads to large negative offset/deviations are more likely to shift the intensity profiles of clusters from “ON” base A towards “OFF” base T at the first intensity channel.

FIG. 28B illustrates the intensity deviations caused by prior trimer context of the clusters that are called as base A and the clusters that are called as base C. For those clusters that are called as base A at a given sequencing cycle, they are segmented into sixty-four subpopulations, each subpopulation representing a particular trimer context identified at prior sequencing cycles proceeding the given sequencing cycle. For each subpopulation, the intensity offset/deviation (“A deviation” at x-axis) at the second intensity channel is calculated by comparing the intensity profiles corresponding to the subpopulation with a mean intensity value. Similarly, the clusters that are called as base C at a given sequencing cycle are segmented into sixty-four subpopulations, each subpopulation representing a particular prior trimer identified at prior sequencing cycles proceeding the given sequencing cycle. For each subpopulation, the intensity offset/deviation (“C deviation” at y-axis) at the second intensity channel is calculated by comparing the intensity profiles corresponding to the subpopulation with a mean intensity value. The deviation caused by prior trimer context when the current base is A is in the range of −0.15 to 0.15 intensity unit, almost ten times more than the deviation caused by prior trimer context when the current base is C. In other words, prior trimer context that leads to large negative deviations are more likely to shift the intensity profiles of clusters from “ON” base A towards “OFF” base C at the second intensity channel.

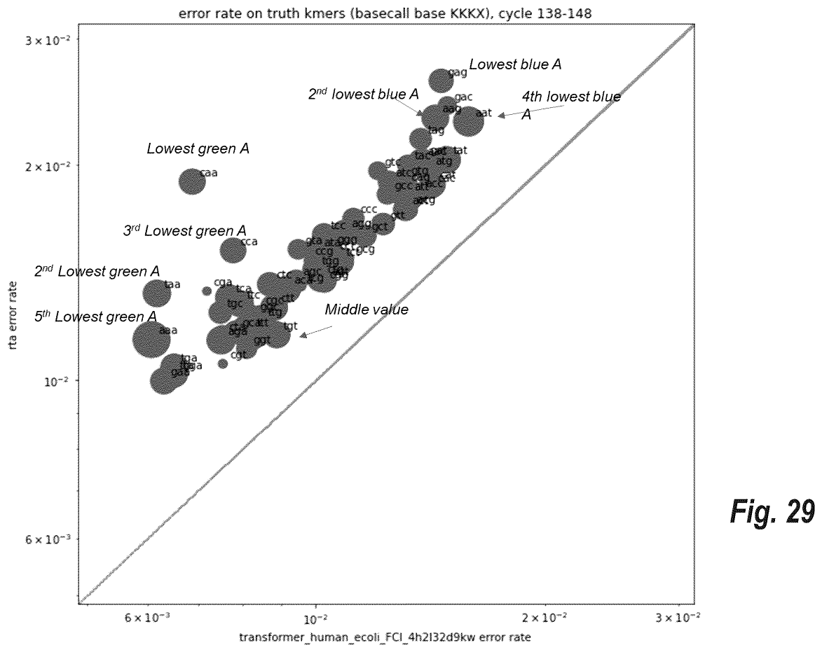

FIG. 29 illustrates the performance results of base calling when correcting for prior base context. Each data point in blue circular form represents clusters that are called as A at a given sequencing cycle and with a particular trimer context identified at prior sequencing cycles proceeding the given sequencing cycle. As annotated, many of the preceding trimers that show the greatest improvement are associated with large deviations in the intensity of base A. For example, the greatest improvement is shown for the CAA trimer at the second intensity channel (e.g., green channel), which is associated with the lowest intensity of base A in the second intensity channel. Similarly, the greatest improvement is shown for the GAG trimer at the first intensity channel (e.g., blue channel), which is associated with the lowest intensity of base A in the second intensity channel.

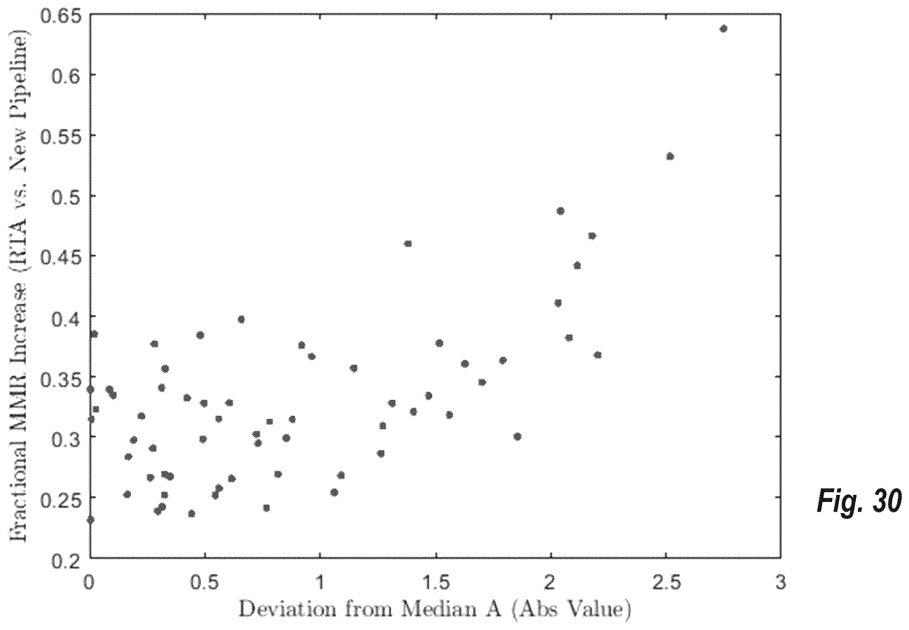

FIG. 30 illustrates fractional MMR improvement when correcting for prior base context, by correlating the fractional MMR increase with deviations from median A intensity in the second intensity channel (e.g., green channel). The fractional MMR increase is calculated by comparing the MMR results using real-time analysis (RTA) without cluster segmentation as benchmark with the technology disclosed herein. The deviation from the median intensity of base A is plotted as an absolute value (x-axis). A negative deviation in the second intensity channel can lead to incorrect calls along the second intensity channel (e.g., A-C decision boundary). A positive deviation in the second intensity channel is associated with a negative deviation in the first intensity channel (e.g., blue channel) which can lead to incorrect calls along the A-T decision boundary. As expected, the greater the prior base context-specific offset/deviation, the greater the fractional MMR increase can be obtained.

### Computer System

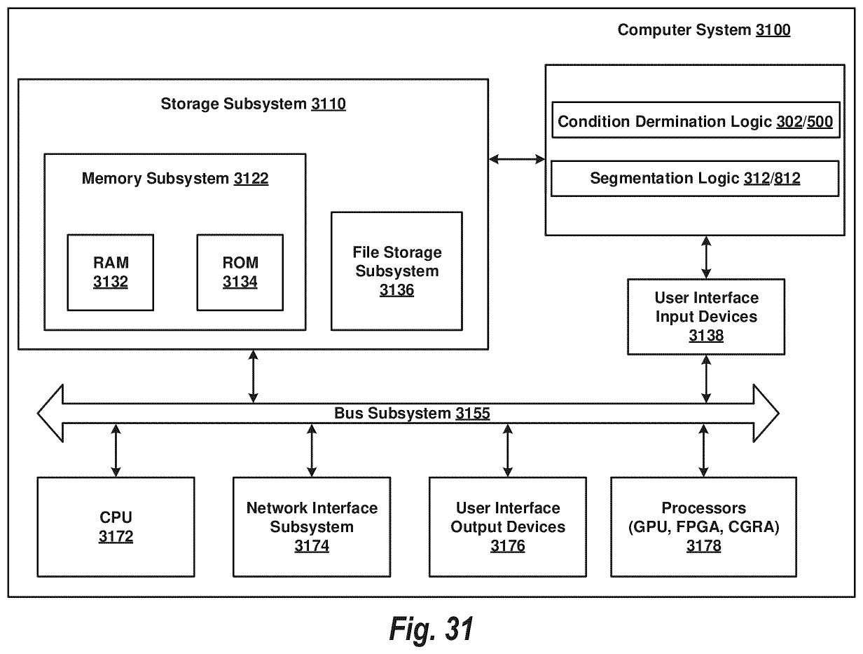

FIG. 31 is a computer system 3100 that can be used to implement the technology disclosed. Computer system 3100 includes at least one central processing unit (CPU) 3172 that communicates with a number of peripheral devices via bus subsystem 3155. These peripheral devices can include a storage subsystem 3110 including, for example, memory devices and a file storage subsystem 3136, user interface input devices 3138, user interface output devices 3176, and a network interface subsystem 3174. The input and output devices allow user interaction with computer system 3100. Network interface subsystem 3174 provides an interface to outside networks, including an interface to corresponding interface devices in other computer systems.

In one implementation, the condition determination logic 302/500 and segmentation logic 312/812 is communicably linked to the storage subsystem 3110 and the user interface input devices 3138.

User interface input devices 3138 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 3100.

User interface output devices 3176 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 3100 to the user or to another machine or computer system.

Storage subsystem 3110 stores programming and data constructs that provide the functionality of some or all of the modules and methods described herein. These software modules are generally executed by processors 3178.

Processors 3178 can be graphics processing units (GPUs), field-programmable gate arrays (FPGAs), application-specific integrated circuits (ASICs), and/or coarse-grained reconfigurable architectures (CGRAs). Processors 3178 can be hosted by a deep learning cloud platform such as Google Cloud Platform™, Xilinx™, and Cirrascale™. Examples of processors 3178 include Google's Tensor Processing Unit (TPU)™, rackmount solutions like GX4 Rackmount Series™, GX15 Rackmount Series™, NVIDIA DGX-1™, Microsoft' Stratix V FPGA™, Graphcore's Intelligent Processor Unit (IPU)™, Qualcomm's Zeroth Platform™ with Snapdragon processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, IBM TrueNorth™, Lambda GPU Server with Testa V100s™, and others.

Memory subsystem 3122 used in the storage subsystem 3110 can include a number of memories including a main random access memory (RAM) 3132 for storage of instructions and data during program execution and a read only memory (ROM) 3134 in which fixed instructions are stored. A file storage subsystem 3136 can provide persistent storage for program and data files, and can include a hard disk drive, a floppy disk drive along with associated removable media, a CD-ROM drive, an optical drive, or removable media cartridges. The modules implementing the functionality of some implementations can be stored by file storage subsystem 3136 in the storage subsystem 3110, or in other machines accessible by the processor.

Bus subsystem 3155 provides a mechanism for letting the various components and subsystems of computer system 3100 communicate with each other as intended. Although bus subsystem 3155 is shown schematically as a single bus, alternative implementations of the bus subsystem can use multiple busses.

Computer system 3100 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 3100 depicted in FIG. 31 is intended only as a specific example for purposes of illustrating the preferred implementations of the present invention. Many other configurations of computer system 3100 are possible having more or less components than the computer system depicted in FIG. 31.

Each of the processors or modules discussed herein may include an algorithm (e.g., instructions stored on a tangible and/or non-transitory computer readable storage medium) or sub-algorithms to perform particular processes. The condition dermination logic 302/500 and segmentation logic 312/812 are illustrated conceptually as a collection of modules, but may be implemented utilizing any combination of dedicated hardware boards, DSPs, processors, etc. Alternatively, the condition dermination logic 302/500 and segmentation logic 312/812 may be implemented utilizing an off-the-shelf PC with a single processor or multiple processors, with the functional operations distributed between the processors. As a further option, the modules described below may be implemented utilizing a hybrid configuration in which some modular functions are performed utilizing dedicated hardware, while the remaining modular functions are performed utilizing an off-the-shelf PC and the like. The modules also may be implemented as software modules within a processing unit.

Various processes and steps of the methods set forth herein can be carried out using a computer. The computer can include a processor that is part of a detection device, networked with a detection device used to obtain the data that is processed by the computer or separate from the detection device. In some implementations, information (e.g., image data) may be transmitted between components of a system disclosed herein directly or via a computer network. A local area network (LAN) or wide area network (WAN) may be a corporate computing network, including access to the Internet, to which computers and computing devices comprising the system are connected. In one implementation, the LAN conforms to the transmission control protocol/internet protocol (TCP/IP) industry standard. In some instances, the information (e.g., image data) is input to a system disclosed herein via an input device (e.g., disk drive, compact disk player, USB port etc.). In some instances, the information is received by loading the information, e.g., from a storage device such as a disk or flash drive.

A processor that is used to run an algorithm or other process set forth herein may comprise a microprocessor. The microprocessor may be any conventional general purpose single- or multi-chip microprocessor such as a Pentium™ processor made by Intel Corporation. A particularly useful computer can utilize an Intel Ivybridge dual-12 core processor, LSI raid controller, having 128 GB of RAM, and 2 TB solid state disk drive. In addition, the processor may comprise any conventional special purpose processor such as a digital signal processor or a graphics processor. The processor typically has conventional address lines, conventional data lines, and one or more conventional control lines.

The implementations disclosed herein may be implemented as a method, apparatus, system or article of manufacture using standard programming or engineering techniques to produce software, firmware, hardware, or any combination thereof. The term “article of manufacture” as used herein refers to code or logic implemented in hardware or computer readable media such as optical storage devices, and volatile or non-volatile memory devices. Such hardware may include, but is not limited to, field programmable gate arrays (FPGAs), application-specific integrated circuits (ASICs), complex programmable logic devices (CPLDs), programmable logic arrays (PLAs), microprocessors, or other similar processing devices. One or more implementations of the technology disclosed, or elements thereof can be implemented in the form of a computer product including a non-transitory computer readable storage medium with computer usable program code for performing the method steps indicated. Furthermore, one or more implementations of the technology disclosed, or elements thereof can be implemented in the form of an apparatus including a memory and at least one processor that is coupled to the memory and operative to perform exemplary method steps. Yet further, in another aspect, one or more implementations of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

### Terminology

As used herein, the term “sequenced data” refer to intensity data (e.g., intensity values) and non-intensity data. In some implementations, the segmentation and conditional base calling are performed on non-intensity data, such as on pH changes induced by the release of hydrogen ions during molecule extension. The pH changes are detected and converted to a voltage change that is proportional to the number of bases incorporated (e.g., in the case of Ion Torrent). Therefore, the sequence data disclosed herein includes voltage signals. In other implementations, the non-intensity data is constructed from nanopore sensing that uses biosensors to measure the disruption in current as an analyte passes through a nanopore or near its aperture while determining the identity of the base. For example, the Oxford Nanopore Technologies (ONT) sequencing is based on the following concept: pass a single strand of DNA (or RNA) through a membrane via a nanopore and apply a voltage difference across the membrane. The nucleotides present in the pore will affect the pore's electrical resistance, so current measurements over time can indicate the sequence of DNA bases passing through the pore. This electrical current signal (the ‘squiggle’ due to its appearance when plotted) is the raw data gathered by an ONT sequencer. These measurements are stored as 16-bit integer data acquisition (DAC) values, taken at e.g., 4 kHz frequency. With a DNA strand velocity of ˜450 base pairs per second, this gives approximately nine raw observations per base on average. This signal is then processed to identify breaks in the open pore signal corresponding to individual reads. These stretches of raw signal are base called—the process of converting DAC values into a sequence of DNA bases. In some implementations, the non-intensity data comprises normalized or scaled DAC values. Therefore, the sequence data disclosed herein can include current signals.

As used herein, the terms “polynucleotide” or “nucleic acids” refer to deoxyribonucleic acid (DNA), but where appropriate the skilled artisan will recognize that the systems and devices herein can also be utilized with ribonucleic acid (RNA). The terms should be understood to include, as equivalents, analogs of either DNA or RNA made from nucleotide analogs. The terms as used herein also encompasses cDNA, that is complementary, or copy, DNA produced from an RNA template, for example by the action of reverse transcriptase.

The single stranded polynucleotide molecules sequenced by the systems and devices herein can have originated in single-stranded form, as DNA or RNA or have originated in double-stranded DNA (dsDNA) form (e.g., genomic DNA fragments, PCR and amplification products and the like). Thus, a single stranded polynucleotide may be the sense or antisense strand of a polynucleotide duplex. Methods of preparation of single stranded polynucleotide molecules suitable for use in the method of the disclosure using standard techniques are well known in the art. The precise sequence of the primary polynucleotide molecules is generally not material to the disclosure, and may be known or unknown. The single stranded polynucleotide molecules can represent genomic DNA molecules (e.g., human genomic DNA) including both intron and exon sequences (coding sequence), as well as non-coding regulatory sequences such as promoter and enhancer sequences.

In some implementations, the nucleic acid to be sequenced through use of the current disclosure is immobilized upon a substrate (e.g., a substrate within a flow cell or one or more beads upon a substrate such as a flow cell, etc.). The term “immobilized” as used herein is intended to encompass direct or indirect, covalent or non-covalent attachment, unless indicated otherwise, either explicitly or by context. In some implementations covalent attachment may be preferred, but generally all that is required is that the molecules (e.g., nucleic acids) remain immobilized or attached to the support under conditions in which it is intended to use the support, for example in applications requiring nucleic acid sequencing.

As indicated above, the present disclosure comprises novel systems and devices for sequencing nucleic acids. As will be apparent to those of skill in the art, references herein to a particular nucleic acid sequence may, depending on the context, also refer to nucleic acid molecules which comprise such nucleic acid sequence. Sequencing of a target fragment means that a read of the chronological order of bases is established. The bases that are read do not need to be contiguous, although this is preferred, nor does every base on the entire fragment have to be sequenced during the sequencing. Sequencing can be carried out using any suitable sequencing technique, wherein nucleotides or oligonucleotides are added successively to a free 3′ hydroxyl group, resulting in synthesis of a polynucleotide chain in the 5′ to 3′ direction. The nature of the nucleotide added is preferably determined after each nucleotide addition. Sequencing techniques using sequencing by ligation, wherein not every contiguous base is sequenced, and techniques such as massively parallel signature sequencing (MPSS) where bases are removed from, rather than added to, the strands on the surface are also amenable to use with the systems and devices of the disclosure.

As described herein, the term “SBS” refers to sequencing-by-synthesis. In SBS, four fluorescently labeled modified nucleotides are used to sequence dense clusters of amplified DNA (possibly millions of clusters) present on the surface of a substrate (e.g., a flow cell). Various additional aspects regarding SBS procedures and methods, which can be utilized with the systems and devices herein, are disclosed in, for example, WO04018497, WO04018493 and U.S. Pat. No. 7,057,026 (nucleotides), WO05024010 and WO06120433 (polymerases), WO05065814 (surface attachment techniques), and WO 9844151, WO06064199 and WO07010251, the contents of each of which are incorporated herein by reference in their entirety.

As used herein, an element or step recited in the singular and proceeded with the word “a” or “an” should be understood as not excluding plural of said elements or steps, unless such exclusion is explicitly stated. Furthermore, references to “one implementation” are not intended to be interpreted as excluding the existence of additional implementations that also incorporate the recited features. Moreover, unless explicitly stated to the contrary, implementations “comprising” or “having” or “including” an element or a plurality of elements having a particular property may include additional elements whether or not they have that property.

In particular implementations, the reaction includes the incorporation of a fluorescently-labeled molecule to an analyte. The analyte may be an oligonucleotide and the fluorescently-labeled molecule may be a nucleotide. The desired reaction may be detected when an excitation light is directed toward the oligonucleotide having the labeled nucleotide, and the fluorophore emits a detectable fluorescent signal. In alternative implementations, the detected fluorescence is a result of chemiluminescence or bioluminescence. A desired reaction may also increase fluorescence (or Förster) resonance energy transfer (FRET), for example, by bringing a donor fluorophore in proximity to an acceptor fluorophore, decrease FRET by separating donor and acceptor fluorophores, increase fluorescence by separating a quencher from a fluorophore or decrease fluorescence by co-locating a quencher and fluorophore.

In some implementations, sensors (e.g., light detectors, photodiodes) are associated with corresponding pixel areas of a sample surface of a biosensor. As such, a pixel area is a geometrical construct that represents an area on the biosensor's sample surface for one sensor (or pixel). A sensor that is associated with a pixel area detects light emissions gathered from the associated pixel area when a desired reaction has occurred at a reaction site or a reaction chamber overlying the associated pixel area. In a flat surface implementation, the pixel areas can overlap. In some cases, a plurality of sensors may be associated with a single reaction site or a single reaction chamber. In other cases, a single sensor may be associated with a group of reaction sites or a group of reaction chambers.

As used herein, a “biosensor” includes a structure having a plurality of reaction sites and/or reaction chambers (or wells). A biosensor may include a solid-state imaging device (e.g., CCD or CMOS imager) and, optionally, a flow cell mounted thereto. The flow cell may include at least one flow channel that is in fluid communication with the reaction sites and/or the reaction chambers. As one specific example, the biosensor is configured to fluidically and electrically couple to a bioassay system. The bioassay system may deliver reactants to the reaction sites and/or the reaction chambers according to a predetermined protocol (e.g., sequencing-by-synthesis) and perform a plurality of imaging events. For example, the bioassay system may direct solutions to flow along the reaction sites and/or the reaction chambers. At least one of the solutions may include four types of nucleotides having the same or different fluorescent labels. The nucleotides may bind to corresponding oligonucleotides located at the reaction sites and/or the reaction chambers. The bioassay system may then illuminate the reaction sites and/or the reaction chambers using an excitation light source (e.g., solid-state light sources, such as light-emitting diodes or LEDs). The excitation light may have a predetermined wavelength or wavelengths, including a range of wavelengths. The excited fluorescent labels provide emission signals that may be captured by the sensors.

In alternative implementations, the biosensor may include electrodes or other types of sensors configured to detect other identifiable properties. For example, the sensors may be configured to detect a change in ion concentration. In another example, the sensors may be configured to detect the ion current flow across a membrane.

As used herein, a “cluster” is a colony of similar or identical molecules or nucleotide sequences or DNA strands. For example, a cluster can be an amplified oligonucleotide or any other group of a polynucleotide or polypeptide with a same or similar sequence. In other implementations, a cluster can be any element or group of elements that occupy a physical area on a sample surface. In implementations, clusters are immobilized to a reaction site and/or a reaction chamber during a base calling cycle.

As used herein, “base calling” identifies a nucleotide base in a nucleic acid sequence. Base calling refers to the process of determining a base call (A, C, G, T) for every cluster at a specific cycle. As an example, base calling can be performed utilizing four-channel, two-channel or one-channel methods and systems described in the incorporated materials of U.S. Patent Application Publication No. 2013/0079232. In particular implementations, a base calling cycle is referred to as a “sampling event.” In one dye and two-channel sequencing protocol, a sampling event comprises two illumination stages in time sequence, such that a pixel signal is generated at each stage. The first illumination stage induces illumination from a given cluster indicating nucleotide bases A and T in a AT pixel signal, and the second illumination stage induces illumination from a given cluster indicating nucleotide bases C and T in a CT pixel signal.

It should be noted that the technology disclosed can be used for base calling on four-channel, two-channel or one-channel sequencing platforms. For example, a two-channel sequencing platform uses a mix of dyes for each base and uses red and green filters for the two images. Clusters seen in red or green images are interpreted as C and T bases, respectively. Clusters observed in both red and green images are interpreted as A bases, while unlabeled clusters identified as G bases. The technology disclosed can segment the population of clusters based on the intensity profiles of clusters captured from both color/intensity channels and apply a mixture of four distributions to the current intensity values of each subpopulation of clusters, wherein the four distributions correspond to four bases A, G, C and T. For a four-channel sequencing platform, each type of bases A, G, C and T has a unique fluorescent dye color; e.g., green to T, red for C, blue for G, and yellow for A. The type of bases with a highest intensity value is identified to be the base call. When base G is called at immediately preceding sequencing cycle, all the intensity values for the following base at current sequencing cycle may be reduced by the “pendant arm” of the fluorophores attached to base G, although the magnitude of reduction may vary among different types of bases. The technology disclosed can segment the population of clusters into subpopulations based on their prior base context to separately base call the clusters in each subpopulation. The technology disclosed can correct the intensity loss caused by the “pendant arm” at each color/intensity channel on a subpopulation-by-subpopulation basis. For example, for each base (i.e., A, G, C and T) that immediately follows base G, the technology disclosed can determine the respective intensity loss (e.g., base-specific offset) at the respective color/intensity channels and correct the intensities accordingly. The corrected intensity values can be used to call the respective bases.

As used herein, “logic” (e.g., condition determination logic, segmentation logic), can be rule-based and implemented in the form of a computer product including a non-transitory computer readable storage medium with computer usable program code for performing the method steps described herein. The “logic” can be implemented in the form of an apparatus including a memory and at least one processor that is coupled to the memory and operative to perform exemplary method steps. The rule-based reassignment and rescaling logics can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media). In one implementation, the logic implements a data processing function. The logic can be a general purpose, single core or multicore, processor with a computer program specifying the function, a digital signal processor with a computer program, configurable logic such as an FPGA with a configuration file, a special purpose circuit such as a state machine, or any combination of these. Also, a computer program product can embody the computer program and configuration file portions of the logic.

In some implementations, a computer-implemented method set forth herein can occur in real time while multiple images of an object are being obtained. Such real time analysis is particularly useful for nucleic acid sequencing applications wherein an array of nucleic acids is subjected to repeated cycles of fluidic and detection steps. Analysis of the sequencing data can often be computationally intensive such that it can be beneficial to perform the methods set forth herein in real time or in the background while other data acquisition or analysis algorithms are in process. Example real time analysis methods that can be used with the present methods are those used for the MiSeq and HiSeq sequencing devices commercially available from Illumina, Inc. (San Diego, Calif.) and/or described in US Pat. App. Pub. No. 2012/0020537 A1, which is incorporated herein by reference.

One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

The detailed description of some implementations will be better understood when read in conjunction with the appended drawings. To the extent that the figures illustrate diagrams of the functional blocks of various implementations, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be standalone programs, may be incorporated as subroutines in an operating system, may be functions in an installed software package, and the like. It should be understood that the various implementations are not limited to the arrangements and instrumentality shown in the drawings.

### Clauses

The technology disclosed, in particularly, the clauses disclosed in this section, can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

One or more implementations and clauses of the technology disclosed or elements thereof can be implemented in the form of a computer product, including a non-transitory computer readable storage medium with computer usable program code for performing the method steps indicated. Furthermore, one or more implementations and clauses of the technology disclosed or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

We disclose the following clauses:
1. A computer-implemented method, including:
segmenting a population of clusters into a plurality of subpopulations of clusters based on one or more prior bases called at one or more prior sequencing cycles of a sequencing run; and
at a current sequencing cycle of the sequencing run:

applying a mixture of four distributions to current sequenced data of each subpopulation of clusters in the plurality of subpopulations of clusters, wherein the four distributions correspond to four bases adenine (A), cytosine (C), guanine (G), and thymine (T), and wherein the current sequenced data is generated at the current sequencing cycle; and

base calling clusters in a particular subpopulation of clusters using a corresponding mixture of four distributions.
2. The computer-implemented method of clause 1, further including resegmenting the population of clusters into the plurality of subpopulations at different intervals in the sequencing run.
3. The computer-implemented method of clause 2, wherein the different intervals correspond to successive sequencing cycles in the sequencing run.
4. The computer-implemented method of clause 2, wherein the different intervals correspond to alternative sequencing cycles in the sequencing run.
5. The computer-implemented method of clause 2, wherein the different intervals correspond to blocks of sequencing cycles in the sequencing run.
6. The computer-implemented method of clause 1, wherein the one or more prior sequencing cycles contiguously precede the current sequencing cycle, and therefore the one or more prior bases called are contiguously preceding base calls.
7. The computer-implemented method of clause 1, wherein the one or more prior sequencing cycles non-contiguously precede the current sequencing cycle, and therefore the one or more prior bases called are non-contiguously preceding base calls.
8. The computer-implemented method of clause 1, further including segmenting the population of clusters into four subpopulations of clusters based on a prior base called at a prior sequencing cycle of the sequencing run,
wherein the four subpopulations correspond to

(1) those clusters in the population of clusters that had an A base call at the prior sequencing cycle,

(2) those clusters in the population of clusters that had a C base call at the prior sequencing cycle,

(3) those clusters in the population of clusters that had a G base call at the prior sequencing cycle, and

(4) those clusters in the population of clusters that had a T base call at the prior sequencing cycle.
9 The computer-implemented method of clause 1, further including segmenting the population of clusters into 4^{k} subpopulations of clusters based on k prior bases called at k prior sequencing cycles of the sequencing run.
10. The computer-implemented method of clause 9, further including segmenting the population of clusters into sixteen subpopulations of clusters based on two prior bases called at two prior sequencing cycles of the sequencing run, and
wherein the sixteen subpopulations correspond to

(1) those clusters in the population of clusters that had AA base calls at the two prior sequencing cycles,

(2) those clusters in the population of clusters that had AC base calls at the two prior sequencing cycles,

(3) those clusters in the population of clusters that had AG base calls at the two prior sequencing cycles,

(4) those clusters in the population of clusters that had AT base calls at the two prior sequencing cycles,

(5) those clusters in the population of clusters that had CA base calls at the two prior sequencing cycles,

(6) those clusters in the population of clusters that had CC base calls at the two prior sequencing cycles,

(7) those clusters in the population of clusters that had CG base calls at the two prior sequencing cycles,

(8) those clusters in the population of clusters that had CT base calls at the two prior sequencing cycles,

(9) those clusters in the population of clusters that had GA base calls at the two prior sequencing cycles,

(10) those clusters in the population of clusters that had GC base calls at the two prior sequencing cycles,

(11) those clusters in the population of clusters that had GG base calls at the two prior sequencing cycles,

(12) those clusters in the population of clusters that had GT base calls at the two prior sequencing cycles,

(13) those clusters in the population of clusters that had TA base calls at the two prior sequencing cycles,

(14) those clusters in the population of clusters that had TC base calls at the two prior sequencing cycles,

(15) those clusters in the population of clusters that had TG base calls at the two prior sequencing cycles, and

(16) those clusters in the population of clusters that had TT base calls at the two prior sequencing cycles.
11. The computer-implemented method of clause 10, further including applying sixteen mixtures of the four distributions to four subpopulations.
12. The computer-implemented method of clause 11, wherein the sixteen mixtures of the four distributions correspond to sixteen centroids.
13. The computer-implemented method of clause 11, wherein the sixteen mixtures of the four distributions correspond to four centroids, with each of the four centroids having four offsets, thereby having a total of sixteen offsets.
14. The computer-implemented method of clause 13, wherein the sixteen offsets are sixteen summary statistics determined from sixteen subpopulation-wise sequenced data.
15. The computer-implemented method of clause 14, wherein the sixteen summary statistics are sixteen sets of channel-specific medians.
16. The computer-implemented method of clause 14, wherein the sixteen summary statistics are sixteen sets of channel-specific means.
17. The computer-implemented method of clause 1, further including segmenting the population of clusters into sixty-four subpopulations of clusters based on three prior bases called at three prior sequencing cycles of the sequencing run, and
wherein the sixty-four subpopulations correspond to

(1) those clusters in the population of clusters that had AAA base calls at the three prior sequencing cycles,

(2) those clusters in the population of clusters that had AAC base calls at the three prior sequencing cycles,

(3) those clusters in the population of clusters that had AAG base calls at the three prior sequencing cycles,

(4) those clusters in the population of clusters that had AAT base calls at the three prior sequencing cycles,

(5) those clusters in the population of clusters that had ACA base calls at the three prior sequencing cycles,

(6) those clusters in the population of clusters that had ACC base calls at the three prior sequencing cycles,

(7) those clusters in the population of clusters that had ACG base calls at the three prior sequencing cycles,

(8) those clusters in the population of clusters that had ACT base calls at the three prior sequencing cycles,

(9) those clusters in the population of clusters that had AGA base calls at the three prior sequencing cycles,

(10) those clusters in the population of clusters that had AGC base calls at the three prior sequencing cycles,

(11) those clusters in the population of clusters that had AGG base calls at the three prior sequencing cycles,

(12) those clusters in the population of clusters that had AGT base calls at the three prior sequencing cycles,

(13) those clusters in the population of clusters that had ATA base calls at the three prior sequencing cycles,

(14) those clusters in the population of clusters that had ATC base calls at the three prior sequencing cycles,

(15) those clusters in the population of clusters that had ATG base calls at the three prior sequencing cycles,

(16) those clusters in the population of clusters that had ATT base calls at the three prior sequencing cycles,

(17) those clusters in the population of clusters that had CAA base calls at the three prior sequencing cycles,

(18) those clusters in the population of clusters that had CAC base calls at the three prior sequencing cycles,

(19) those clusters in the population of clusters that had CAG base calls at the three prior sequencing cycles,

(20) those clusters in the population of clusters that had CAT base calls at the three prior sequencing cycles,

(21) those clusters in the population of clusters that had CCA base calls at the three prior sequencing cycles,

(22) those clusters in the population of clusters that had CCC base calls at the three prior sequencing cycles,

(23) those clusters in the population of clusters that had CCG base calls at the three prior sequencing cycles,

(24) those clusters in the population of clusters that had CCT base calls at the three prior sequencing cycles,

(25) those clusters in the population of clusters that had CGA base calls at the three prior sequencing cycles,

(26) those clusters in the population of clusters that had CGC base calls at the three prior sequencing cycles,

(27) those clusters in the population of clusters that had CGG base calls at the three prior sequencing cycles,

(28) those clusters in the population of clusters that had CGT base calls at the three prior sequencing cycles,

(29) those clusters in the population of clusters that had CTA base calls at the three prior sequencing cycles,

(30) those clusters in the population of clusters that had CTC base calls at the three prior sequencing cycles,

(31) those clusters in the population of clusters that had CTG base calls at the three prior sequencing cycles,

(32) those clusters in the population of clusters that had CTT base calls at the three prior sequencing cycles,

(33) those clusters in the population of clusters that had GAA base calls at the three prior sequencing cycles,

(34) those clusters in the population of clusters that had GAC base calls at the three prior sequencing cycles,

(35) those clusters in the population of clusters that had GAG base calls at the three prior sequencing cycles,

(36) those clusters in the population of clusters that had GAT base calls at the three prior sequencing cycles,

(37) those clusters in the population of clusters that had GCA base calls at the three prior sequencing cycles,

(38) those clusters in the population of clusters that had GCC base calls at the three prior sequencing cycles,

(39) those clusters in the population of clusters that had GCG base calls at the three prior sequencing cycles,

(40) those clusters in the population of clusters that had GCT base calls at the three prior sequencing cycles,

(41) those clusters in the population of clusters that had GGA base calls at the three prior sequencing cycles,

(42) those clusters in the population of clusters that had GGC base calls at the three prior sequencing cycles,

(43) those clusters in the population of clusters that had GGG base calls at the three prior sequencing cycles,

(44) those clusters in the population of clusters that had GGT base calls at the three prior sequencing cycles,

(45) those clusters in the population of clusters that had GTA base calls at the three prior sequencing cycles,

(46) those clusters in the population of clusters that had GTC base calls at the three prior sequencing cycles,

(47) those clusters in the population of clusters that had GTG base calls at the three prior sequencing cycles,

(48) those clusters in the population of clusters that had GTT base calls at the three prior sequencing cycles,

(49) those clusters in the population of clusters that had TAA base calls at the three prior sequencing cycles,

(50) those clusters in the population of clusters that had TAC base calls at the three prior sequencing cycles,

(51) those clusters in the population of clusters that had TAG base calls at the three prior sequencing cycles,

(52) those clusters in the population of clusters that had TAT base calls at the three prior sequencing cycles,

(53) those clusters in the population of clusters that had TCA base calls at the three prior sequencing cycles,

(54) those clusters in the population of clusters that had TCC base calls at the three prior sequencing cycles,

(55) those clusters in the population of clusters that had TCG base calls at the three prior sequencing cycles,

(56) those clusters in the population of clusters that had TCT base calls at the three prior sequencing cycles,

(57) those clusters in the population of clusters that had TGA base calls at the three prior sequencing cycles,

(58) those clusters in the population of clusters that had TGC base calls at the three prior sequencing cycles,

(59) those clusters in the population of clusters that had TGG base calls at the three prior sequencing cycles,

(60) those clusters in the population of clusters that had TGT base calls at the three prior sequencing cycles,

(61) those clusters in the population of clusters that had TTA base calls at the three prior sequencing cycles,

(62) those clusters in the population of clusters that had TTC base calls at the three prior sequencing cycles,

(63) those clusters in the population of clusters that had TTG base calls at the three prior sequencing cycles, and

(64) those clusters in the population of clusters that had TTT base calls at the three prior sequencing cycles.
18. The computer-implemented method of clause 17, further including applying sixty-four mixtures of the four distributions to the sixty-four subpopulations.
19. The computer-implemented method of clause 18, wherein the sixty-four mixtures of the four distributions correspond to sixty-four centroids.
20. The computer-implemented method of clause 19, wherein the sixty-four mixtures of the four distributions correspond to four centroids, with each of the four centroids having sixteen offsets, thereby having a total of sixty-four offsets.
21. The computer-implemented method of clause 20, wherein the sixty-four offsets are sixty-four summary statistics determined from sixty-four subpopulation-wise sequenced data.
22. The computer-implemented method of clause 21, wherein the sixty-four summary statistics are sixty-four sets of channel-specific medians.
23. The computer-implemented method of clause 21, wherein the sixty-four summary statistics are sixty-four sets of channel-specific means.
24. The computer-implemented method of clause 1, further including segmenting the population of clusters into the plurality of subpopulations based on one or more right and left flanking bases called at one or more right and left flanking sequencing cycles of the sequencing run.
25. The computer-implemented method of clause 24, further including segmenting the population of clusters into 4^{(r+l)} subpopulations of clusters, where r is a number of succeeding bases called at r succeeding sequencing cycles of the sequencing run, and l is a number of prior bases called at l prior sequencing cycles of the sequencing run.
26. The computer-implemented method of clause 1, further including segmenting the population of clusters into the plurality of subpopulations based on different signal-to-noise ratio profiles detected in sequenced data of the population of clusters.
27. The computer-implemented method of clause 26, further including segmenting the population of clusters into p subpopulations of clusters, where p is a number of the different signal-to-noise ratio profiles.
28. The computer-implemented method of clause 27, wherein the different signal-to-noise ratio profiles are determined for different signal-to-noise ratio ranges.
29. The computer-implemented method of clause 1, further including segmenting the population of clusters into the plurality of subpopulations based on different library types from which the population of clusters is sourced.
30. The computer-implemented method of clause 29, further including segmenting the population of clusters into s subpopulations of clusters, where s is a number of the different library types.
31. The computer-implemented method of clause 29, further including segmenting the population of clusters into the plurality of subpopulations based on different insert lengths detected for the different library types.
32. The computer-implemented method of clause 31, further including segmenting the population of clusters into i subpopulations of clusters, where i is a number of the different insert lengths.
33. The computer-implemented method of clause 1, further including segmenting the population of clusters into the plurality of subpopulations based on different values of variation correction coefficients determined to correct variations in sequenced data of the population of clusters.
34. The computer-implemented method of clause 32, further including segmenting the population of clusters into v subpopulations of clusters, where v is a number of different variation correction coefficients.
35. The computer-implemented method of clause 34, wherein the variation correction coefficients include channel-specific amplification coefficients that correct scale variations in the sequenced data of the population of clusters.
36. The computer-implemented method of clause 34, wherein the variation correction coefficients include channel-specific offset coefficients that correct shift variations in the sequenced data of the population of clusters.
37. The computer-implemented method of clause 1, further including segmenting the population of clusters into the plurality of subpopulations based on different spatial configurations of the population of clusters on a biosensor.
38. The computer-implemented method of clause 37, wherein the different spatial configurations include tile locations, sub-tile locations, surface locations, section locations, lane locations, lane group locations, swath locations, and/or swath group locations.
39. The computer-implemented method of clause 1, further including segmenting the population of clusters into the plurality of subpopulations based on different raw intensity profiles detected in sequenced data of the population of clusters.
40. The computer-implemented method of clause 39, further including segmenting the population of clusters into j subpopulations of clusters, where j is a number of the different raw intensity profiles.
41. The computer-implemented method of clause 1, further including segmenting the population of clusters into the plurality of subpopulations based on different sample types from which the population of clusters is sourced.
42. The computer-implemented method of clause 41, further including segmenting the population of clusters into x subpopulations of clusters, where x is a number of the different sample types.
43. The computer-implemented method of clause 1, further including segmenting the population of clusters into the plurality of subpopulations based on different index reads used during the sequencing run.
44. The computer-implemented method of clause 43, further including segmenting the population of clusters into y subpopulations of clusters, where y is a number of the different index reads.
45. The computer-implemented method of clause 1, further including segmenting the population of clusters into the plurality of subpopulations based on different signal variation types detected in sequenced data of the population of clusters.
46. The computer-implemented method of clause 45, further including segmenting the population of clusters into n subpopulations of clusters, where n is a number of the different signal variation types.
47. The computer-implemented method of clause 1, wherein each distribution in the four distributions has a mean and a covariance.
48. The computer-implemented method of clause 1, further including applying the mixture of four distributions using one or more logics from a group consisting of: a k-means clustering algorithm, a k-means-like clustering algorithm, expectation maximization, and a histogram based method.
49. The computer-implemented method of clause 1, wherein the current sequenced data is detected using charge-coupled device (CCD) sensors.
50. The computer-implemented method of clause 1, wherein the current sequenced data is detected using complementary metal-oxide-semiconductor (CMOS) sensors.
51. The computer-implemented method of clause 1, wherein the current sequenced data includes intensity signals.
52. The computer-implemented method of clause 1, wherein the current sequenced data includes voltage signals.
53. The computer-implemented method of clause 1, wherein the current sequenced data includes current signals.
54. The computer-implemented method of clause 1, further including segmenting the population of clusters into the plurality of subpopulations of clusters based on one or more subsequent base calls at one or more subsequent sequencing cycles of the sequencing run.
55. The computer-implemented method of clause 54, wherein the one or more subsequent sequencing cycles contiguously succeed the current sequencing cycle, and therefore the one or more subsequent base calls are contiguously succeeding base calls.
56. The computer-implemented method of clause 55, wherein the one or more subsequent sequencing cycles non-contiguously succeed the current sequencing cycle, and therefore the one or more subsequent base calls are non-contiguously succeeding base calls.
57. The computer-implemented method of clause 1, further including segmenting the population of clusters into the plurality of subpopulations of clusters only once during the sequencing run.
58. The computer-implemented method of clause 57, further including segmenting the population of clusters into the plurality of subpopulations of clusters only at a first sequencing cycle of the sequencing run.
59. A computer-implemented method, including:
segmenting a population of clusters into a plurality of subpopulations of clusters based on one or more segmentation conditions; and
at a current sequencing cycle of a sequencing run:

applying a mixture of four distributions to sequenced data of each subpopulation of clusters in the plurality of subpopulations of clusters, wherein the four distributions correspond to four bases adenine (A), cytosine (C), guanine (G), and thymine (T), and wherein current sequenced data is generated at the current sequencing cycle; and

base calling clusters in a particular subpopulation of clusters using a corresponding mixture of four distributions.
60. The computer-implemented method of clause 59, wherein the one or more segmentation conditions include previous base calls segmentation condition.
61. The computer-implemented method of clause 59, wherein the one or more segmentation conditions include succeeding base calls segmentation condition.
62. The computer-implemented method of clause 59, wherein the one or more segmentation conditions include right and left flanking base calls segmentation condition.
63. The computer-implemented method of clause 59, wherein the one or more segmentation conditions include different signal-to-noise ratio profiles segmentation condition.
64. The computer-implemented method of clause 59, wherein the one or more segmentation conditions include different library types segmentation condition.
65. The computer-implemented method of clause 59, wherein the one or more segmentation conditions include different insert lengths segmentation condition.
66. The computer-implemented method of clause 59, wherein the one or more segmentation conditions include different values of variation correction coefficients segmentation condition.
67. The computer-implemented method of clause 59, wherein the one or more segmentation conditions include different spatial configurations segmentation condition.
68. The computer-implemented method of clause 59, wherein the one or more segmentation conditions include different raw intensity profiles segmentation condition.
69. The computer-implemented method of clause 59, wherein the one or more segmentation conditions include different sample types segmentation condition.
70. The computer-implemented method of clause 59, wherein the one or more segmentation conditions include different index reads segmentation condition.
71. The computer-implemented method of clause 59, wherein the one or more segmentation conditions include different signal variation types segmentation condition.
72. The computer-implemented method of clause 59, further including resegmenting the population of clusters into the plurality of subpopulations at different intervals in the sequencing run.
73. The computer-implemented method of clause 72, wherein the different intervals correspond to successive sequencing cycles in the sequencing run.
74. The computer-implemented method of clause 72, wherein the different intervals correspond to alternative sequencing cycles in the sequencing run.
75. The computer-implemented method of clause 72, wherein the different intervals correspond to blocks of sequencing cycles in the sequencing run.
76. The computer-implemented method of clause 59, further including segmenting the population of clusters into the plurality of subpopulations of clusters only once during the sequencing run.
77. The computer-implemented method of clause 59, further including segmenting the population of clusters into the plurality of subpopulations of clusters only at a first sequencing cycle of the sequencing run.
78. A computer-implemented method, including:
at a current sequencing cycle of a sequencing run:

accessing current sequenced data for a population of clusters, wherein the current sequenced data is generated at the current sequencing cycle;

accessing prior sequenced data for the population of clusters, wherein the prior sequenced data is generated at k prior sequencing cycles of the sequencing run, where K≥1;

applying 4k+1 mixtures of four distributions to the current sequenced data and the prior sequenced data,
wherein the four distributions correspond to four bases adenine (A), cytosine (C), guanine (G), and thymine (T), and
wherein the 4^{k+1} mixtures correspond to 4^{k+1} permutations of (i) k prior bases called at the k prior sequencing cycles based on the prior sequenced data and (ii) a corresponding one of the four bases A, C, G, and T; and

base calling the population of clusters using a mixture of four nested distributions.
79. The computer-implemented method of clause 78, wherein the 4^{k+1} permutations are permutations with repetition.
80. The computer-implemented method of clause 78, wherein the prior sequenced data is generated at a prior sequencing cycle of the sequencing run, with k=1, k+1=2, and 4²=16.
81. The computer-implemented method of clause 80, wherein sixteen mixtures of the four distributions correspond to sixteen permutations of: (i) a prior base called at the prior sequencing cycle based on the prior sequenced data and (ii) the corresponding one of the four bases A, C, G, and T, including:
(1) AA,
(2) CA,
(3) GA,
(4) TA,
(5) AC,
(6) CC,
(7) GC,
(8) TC,
(9) AG,
(10) CG,
(11) GG,
(12) TG,
(13) AT,
(14) CT
(15) GT, and
(16) TT.
82. The computer-implemented method of clause 78, wherein the prior sequenced data is generated at two prior sequencing cycles of the sequencing run, with k=2, k+1=3, and 4³=64.
83. The computer-implemented method of clause 78, wherein sixty-four mixtures of the four distributions correspond to sixty-four permutations of: (i) two prior bases called at two prior sequencing cycles based on the prior sequenced data and (ii) the corresponding one of the four bases A, C, G, and T, including:
(1) AAA
(2) ACA
(3) AGA
(4) ATA
(5) CAA
(6) CCA
(7) CGA
(8) CTA
(9) GAA
(10) GCA
(11) GGA
(12) GTA
(13) TAA
(14) TCA
(15) TGA
(16) TTA
(17) AAC
(18) ACC
(19) AGC
(20) ATC
(21) CAC
(22) CCC
(23) CGC
(24) CTC
(25) GAC
(26) GCC
(27) GGC
(28) GTC
(29) TAC
(30) TCC
(31) TGC
(32) TTC
(33) AAG
(34) ACG
(35) AGG
(36) ATG
(37) CAG
(38) CCG
(39) CGG
(40) CTG
(41) GAG
(42) GCG
(43) GGG
(44) GTG
(45) TAG
(46) TCG
(47) TGG
(48) TTG
(49) AAT
(50) ACT
(51) AGT
(52) ATT
(53) CAT
(54) CCT
(55) CGT
(56) CTT
(57) GAT
(58) GCT
(59) GGT
(60) GTT
(61) TAT
(62) TCT
(63) TGT
(64) TTT.

While the present invention is disclosed by reference to the preferred implementations and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims.

What is claimed is:

1. A system comprising:
at least one processor; and
at least a non-transitory computer readable storage medium comprising instructions that, when executed by the at least one processor, cause the system to:
segment a population of clusters into a plurality of subpopulations of clusters based on one or more prior bases called at one or more prior sequencing cycles of a sequencing run; and
at a current sequencing cycle of the sequencing run:
apply a mixture of four distributions to current sequenced data of each subpopulation of clusters in the plurality of subpopulations of clusters, wherein the four distributions correspond to four bases adenine (A), cytosine (C), guanine (G), and thymine (T), and wherein the current sequenced data is generated at the current sequencing cycle; and
base call clusters in a particular subpopulation of clusters using a corresponding mixture of four distributions.

2. The system of claim 1, further comprising instructions that, when executed by the at least one processor, cause the system to resegment the population of clusters into the plurality of subpopulations at different intervals in the sequencing run.

3. The system of claim 2, wherein the different intervals correspond to successive sequencing cycles in the sequencing run, alternative sequencing cycles in the sequencing run, or blocks of sequencing cycles in the sequencing run.

4. The system of claim 1, wherein the one or more prior sequencing cycles contiguously precede the current sequencing cycle, and therefore the one or more prior bases called are contiguously preceding base calls.

5. The system of claim 1, wherein the one or more prior sequencing cycles non-contiguously precede the current sequencing cycle, and therefore the one or more prior bases called are non-contiguously preceding base calls.

6. The system of claim 1, further comprising instructions that, when executed by the at least one processor, cause the system to segment the population of clusters into four subpopulations of clusters based on a prior base called at a prior sequencing cycle of the sequencing run,
wherein the four subpopulations correspond to:
(1) those clusters in the population of clusters that had an A base call at the prior sequencing cycle,
(2) those clusters in the population of clusters that had a C base call at the prior sequencing cycle,
(3) those clusters in the population of clusters that had a G base call at the prior sequencing cycle, and
(4) those clusters in the population of clusters that had a T base call at the prior sequencing cycle.

7. The system of claim 1, further comprising instructions that, when executed by the at least one processor, cause the system to segment the population of clusters into 4^{k} subpopulations of clusters based on k prior bases called at k prior sequencing cycles of the sequencing run.

8. The system of claim 1, further comprising instructions that, when executed by the at least one processor, cause the system to segment the population of clusters into sixteen subpopulations of clusters based on two prior bases called at two prior sequencing cycles of the sequencing run, and
wherein the sixteen subpopulations correspond to:
(1) those clusters in the population of clusters that had AA base calls at the two prior sequencing cycles,
(2) those clusters in the population of clusters that had AC base calls at the two prior sequencing cycles,
(3) those clusters in the population of clusters that had AG base calls at the two prior sequencing cycles,
(4) those clusters in the population of clusters that had AT base calls at the two prior sequencing cycles,
(5) those clusters in the population of clusters that had CA base calls at the two prior sequencing cycles,
(6) those clusters in the population of clusters that had CC base calls at the two prior sequencing cycles,
(7) those clusters in the population of clusters that had CG base calls at the two prior sequencing cycles,
(8) those clusters in the population of clusters that had CT base calls at the two prior sequencing cycles,
(9) those clusters in the population of clusters that had GA base calls at the two prior sequencing cycles,
(10) those clusters in the population of clusters that had GC base calls at the two prior sequencing cycles,
(11) those clusters in the population of clusters that had GG base calls at the two prior sequencing cycles,
(12) those clusters in the population of clusters that had GT base calls at the two prior sequencing cycles,
(13) those clusters in the population of clusters that had TA base calls at the two prior sequencing cycles,
(14) those clusters in the population of clusters that had TC base calls at the two prior sequencing cycles,
(15) those clusters in the population of clusters that had TG base calls at the two prior sequencing cycles, and
(16) those clusters in the population of clusters that had TT base calls at the two prior sequencing cycles.

9. The system of claim 1, further comprising instructions that, when executed by the at least one processor, cause the system to segment the population of clusters into sixty-four subpopulations of clusters based on three prior bases called at three prior sequencing cycles of the sequencing run, and
wherein the sixty-four subpopulations correspond to:
(1) those clusters in the population of clusters that had AAA base calls at the three prior sequencing cycles,
(2) those clusters in the population of clusters that had AAC base calls at the three prior sequencing cycles,
(3) those clusters in the population of clusters that had AAG base calls at the three prior sequencing cycles,
(4) those clusters in the population of clusters that had AAT base calls at the three prior sequencing cycles,
(5) those clusters in the population of clusters that had ACA base calls at the three prior sequencing cycles,
(6) those clusters in the population of clusters that had ACC base calls at the three prior sequencing cycles,
(7) those clusters in the population of clusters that had ACG base calls at the three prior sequencing cycles,
(8) those clusters in the population of clusters that had ACT base calls at the three prior sequencing cycles,
(9) those clusters in the population of clusters that had AGA base calls at the three prior sequencing cycles,
(10) those clusters in the population of clusters that had AGC base calls at the three prior sequencing cycles,
(11) those clusters in the population of clusters that had AGG base calls at the three prior sequencing cycles,
(12) those clusters in the population of clusters that had AGT base calls at the three prior sequencing cycles,
(13) those clusters in the population of clusters that had ATA base calls at the three prior sequencing cycles,
(14) those clusters in the population of clusters that had ATC base calls at the three prior sequencing cycles,
(15) those clusters in the population of clusters that had ATG base calls at the three prior sequencing cycles,
(16) those clusters in the population of clusters that had ATT base calls at the three prior sequencing cycles,
(17) those clusters in the population of clusters that had CAA base calls at the three prior sequencing cycles,
(18) those clusters in the population of clusters that had CAC base calls at the three prior sequencing cycles,
(19) those clusters in the population of clusters that had CAG base calls at the three prior sequencing cycles,
(20) those clusters in the population of clusters that had CAT base calls at the three prior sequencing cycles,
(21) those clusters in the population of clusters that had CCA base calls at the three prior sequencing cycles,
(22) those clusters in the population of clusters that had CCC base calls at the three prior sequencing cycles,
(23) those clusters in the population of clusters that had CCG base calls at the three prior sequencing cycles,
(24) those clusters in the population of clusters that had CCT base calls at the three prior sequencing cycles,
(25) those clusters in the population of clusters that had CGA base calls at the three prior sequencing cycles,
(26) those clusters in the population of clusters that had CGC base calls at the three prior sequencing cycles,
(27) those clusters in the population of clusters that had CGG base calls at the three prior sequencing cycles,
(28) those clusters in the population of clusters that had CGT base calls at the three prior sequencing cycles,
(29) those clusters in the population of clusters that had CTA base calls at the three prior sequencing cycles,
(30) those clusters in the population of clusters that had CTC base calls at the three prior sequencing cycles,
(31) those clusters in the population of clusters that had CTG base calls at the three prior sequencing cycles,
(32) those clusters in the population of clusters that had CTT base calls at the three prior sequencing cycles,
(33) those clusters in the population of clusters that had GAA base calls at the three prior sequencing cycles,
(34) those clusters in the population of clusters that had GAC base calls at the three prior sequencing cycles,
(35) those clusters in the population of clusters that had GAG base calls at the three prior sequencing cycles,
(36) those clusters in the population of clusters that had GAT base calls at the three prior sequencing cycles,
(37) those clusters in the population of clusters that had GCA base calls at the three prior sequencing cycles,
(38) those clusters in the population of clusters that had GCC base calls at the three prior sequencing cycles,
(39) those clusters in the population of clusters that had GCG base calls at the three prior sequencing cycles,
(40) those clusters in the population of clusters that had GCT base calls at the three prior sequencing cycles,
(41) those clusters in the population of clusters that had GGA base calls at the three prior sequencing cycles,
(42) those clusters in the population of clusters that had GGC base calls at the three prior sequencing cycles,
(43) those clusters in the population of clusters that had GGG base calls at the three prior sequencing cycles,
(44) those clusters in the population of clusters that had GGT base calls at the three prior sequencing cycles,
(45) those clusters in the population of clusters that had GTA base calls at the three prior sequencing cycles,
(46) those clusters in the population of clusters that had GTC base calls at the three prior sequencing cycles,
(47) those clusters in the population of clusters that had GTG base calls at the three prior sequencing cycles,
(48) those clusters in the population of clusters that had GTT base calls at the three prior sequencing cycles,
(49) those clusters in the population of clusters that had TAA base calls at the three prior sequencing cycles,
(50) those clusters in the population of clusters that had TAC base calls at the three prior sequencing cycles,
(51) those clusters in the population of clusters that had TAG base calls at the three prior sequencing cycles,
(52) those clusters in the population of clusters that had TAT base calls at the three prior sequencing cycles,
(53) those clusters in the population of clusters that had TCA base calls at the three prior sequencing cycles,
(54) those clusters in the population of clusters that had TCC base calls at the three prior sequencing cycles,
(55) those clusters in the population of clusters that had TCG base calls at the three prior sequencing cycles,
(56) those clusters in the population of clusters that had TCT base calls at the three prior sequencing cycles,
(57) those clusters in the population of clusters that had TGA base calls at the three prior sequencing cycles,
(58) those clusters in the population of clusters that had TGC base calls at the three prior sequencing cycles,
(59) those clusters in the population of clusters that had TGG base calls at the three prior sequencing cycles,
(60) those clusters in the population of clusters that had TGT base calls at the three prior sequencing cycles,
(61) those clusters in the population of clusters that had TTA base calls at the three prior sequencing cycles,
(62) those clusters in the population of clusters that had TTC base calls at the three prior sequencing cycles,
(63) those clusters in the population of clusters that had TTG base calls at the three prior sequencing cycles, and
(64) those clusters in the population of clusters that had TTT base calls at the three prior sequencing cycles.

10. A non-transitory computer readable storage medium comprising instructions that, when executed by at least one processor, cause a system to:
segment a population of clusters into a plurality of subpopulations of clusters based on one or more prior bases called at one or more prior sequencing cycles of a sequencing run; and
at a current sequencing cycle of the sequencing run:
apply a mixture of four distributions to current sequenced data of each subpopulation of clusters in the plurality of subpopulations of clusters, wherein the four distributions correspond to four bases adenine (A), cytosine (C), guanine (G), and thymine (T), and wherein the current sequenced data is generated at the current sequencing cycle; and
base call clusters in a particular subpopulation of clusters using a corresponding mixture of four distributions.

11. The non-transitory computer readable storage medium of claim 10, further comprising instructions that, when executed by the at least one processor, cause the system to segment the population of clusters into the plurality of subpopulations based on one or more right and left flanking bases called at one or more right and left flanking sequencing cycles of the sequencing run.

12. The non-transitory computer readable storage medium of claim 11, further comprising instructions that, when executed by the at least one processor, cause the system to segment the population of clusters into 4^{(r+l)} subpopulations of clusters, where r is a number of succeeding bases called at r succeeding sequencing cycles of the sequencing run, and l is a number of prior bases called at/prior sequencing cycles of the sequencing run.

13. The non-transitory computer readable storage medium of claim 10, further comprising instructions that, when executed by the at least one processor, cause the system to segment the population of clusters into the plurality of subpopulations based on different signal-to-noise ratio profiles detected in sequenced data of the population of clusters.

14. The non-transitory computer readable storage medium of claim 13, further comprising instructions that, when executed by the at least one processor, cause the system to segment the population of clusters into p subpopulations of clusters, where p is a number of the different signal-to-noise ratio profiles.

15. The non-transitory computer readable storage medium of claim 10, further comprising instructions that, when executed by the at least one processor, cause the system to segment the population of clusters into the plurality of subpopulations based on different library types from which the population of clusters is sourced.

16. The non-transitory computer readable storage medium of claim 15, further comprising instructions that, when executed by the at least one processor, cause the system to segment the population of clusters into the plurality of subpopulations based on different insert lengths detected for the different library types.

17. A computer-implemented method, including:
segmenting a population of clusters into a plurality of subpopulations of clusters based on one or more prior bases called at one or more prior sequencing cycles of a sequencing run; and
at a current sequencing cycle of the sequencing run:
applying a mixture of four distributions to current sequenced data of each subpopulation of clusters in the plurality of subpopulations of clusters, wherein the four distributions correspond to four bases adenine (A), cytosine (C), guanine (G), and thymine (T), and wherein the current sequenced data is generated at the current sequencing cycle; and
base calling clusters in a particular subpopulation of clusters using a corresponding mixture of four distributions.

18. The computer-implemented method of claim 17, further including segmenting the population of clusters into the plurality of subpopulations based on different values of variation correction coefficients determined to correct variations in sequenced data of the population of clusters.

19. The computer-implemented method of claim 17, further including segmenting the population of clusters into the plurality of subpopulations based on different spatial configurations of the population of clusters on a biosensor.

20. The computer-implemented method of claim 19, wherein the different spatial configurations include tile locations, sub-tile locations, surface locations, section locations, lane locations, lane group locations, swath locations, and/or swath group locations.
